(19) United States
(12) Patent Application Publication
(10) Pub. No.: US 2011/0264139 A1
(43) Pub. Date: Oct. 27, 2011

(54) SUTURES AND FIBROSING AGENTS

(75) Inventors: William L. HUNTER, Vancouver (CA); Rui Avelar, Vancouver (CA); David Gravett, Mountain View, CA (US); Philip M. Toleikis, Vancouver (CA); Arpita Maiti, Vancouver (CA)

(73) Assignee: Angiotech Pharmaceuticals, Inc., Vancouver (CA)

(21) Appl. No.: 13/085,140

(22) Filed: Apr. 12, 2011

Related U.S. Application Data
(63) Continuation of application No. 11/669,097, filed on Jan. 30, 2007, now abandoned.
(60) Provisional application No. 60/763,682, filed on Jan. 30, 2006.

(51) Int. Cl. A61B 17/04; B23P 11/00

(52) U.S. Cl. 606/228; 29/428

(57) ABSTRACT

Sutures are used in combination with fibrosing agents to induce or stimulate fibrosis that between the sutures and the host tissues into which the sutures are inserted. Compositions and methods are described for use in lifting tissue, closing wound, and other applications.

### CROSS-REFERENCE TO RELATED APPLICATIONS

This application is a continuation of U.S. patent application Ser. No. 11/669,097 filed Jan. 30, 2007; which application claims the benefit under 35 U.S.C. §119(e) of U.S. Provisional Patent Application No. 60/763,682 filed Jan. 30, 2006, which applications are incorporated herein by reference in their entirety.

### BACKGROUND OF THE INVENTION

1. Field of the Invention

The present invention relates generally to pharmaceutical agents and compositions for administration in association with sutures (e.g., self-retaining sutures). More specifically, the present invention relates to compositions and methods for preparing sutures that induce or stimulate a fibrotic response between the sutures and the tissue in contact with the suture material.

2. Description of the Related Art

Sutures are currently the material of choice for wound closure. However, complications associated with knots when using conventional sutures are well known. Such complications include: suture breakage, knot slippage, suture extrusion, infection, dehiscence and excessive inflammatory response leading to ischemia and scarring. Attempts to overcome these deficiencies with knotless sutures in the past have gained little clinical success. More recently, the development of self-retaining (such as barbed) sutures has been reported.

Self-retaining sutures are used to more firmly adhere to, and anchor into, the tissue in which they are placed so as to prevent slippage in wound closure and to lift the skin and subcutaneous tissue during reconstructive or cosmetic procedures (such as face lifts, breast lifts and related interventions). In addition, a self-retaining suture can be used to sew fragile tissues (such as muschle, spleen, renal and liver) to prevent the suture from breaking through such tissues. However, the tensile strength of a self-retaining suture may be less than a conventional suture of equivalent size if the imparting of the retainer structure onto the body of the suture reduces its effective diameter. Also, the suture material can come loose from the tissue with time leading to tissue slippage and loss of the cosmetic benefits in a “tissue lift” procedure. Accordingly, there is a need for sutures that are more effective for wound repair and/or enhance the adherence of plain or self-retaining sutures to the surrounding tissue.

### BRIEF SUMMARY OF THE INVENTION

Briefly stated, the present invention provides sutures (including plain and self-retaining sutures) that comprise a fibrosing agent, as well as methods for making and using such sutures. In addition, the present invention also provides compositions that comprise fibrosing agents and methods for using such compositions in combination with sutures (including plain and self-retaining sutures) in various applications (e.g., tissue reposition and wound closure).

In one aspect, the present invention provides a self-retaining suture comprising a fibrosing agent. In a related aspect, the present invention provides a self-retaining suture connector comprising a fibrosing agent. In another related aspect, the present invention provides a suture anchor that comprises a fibrosing agent.

In another aspect, the present invention provides a method for making a fibrosis-inducing self-retaining suture comprising: combining a suture with a fibrosing agent, wherein the fibrosing agent induces or stimulates a fibrotic response between the suture and a patient in which the suture is implanted.

In another aspect, the present invention provides a method for repositioning tissue comprising: implanting a suture into a tissue in need thereof, and moving the suture so that the tissue into which the suture is implanted is repositioned, wherein the suture comprises a fibrosing agent that induces or stimulates a fibrotic response between the suture and the tissue in which the suture is implanted.

In another aspect, the present invention provides a method for facelifting comprising: implanting a suture that comprises a fibrosing agent into the subepidermal tissue of the face, and moving the suture so that the dermal tissue of the face is uplifted.

In another aspect, the present invention provides a method for closing a wound comprising: joining two surfaces or edges of a wound together with a suture that comprises a fibrosing agent, wherein the fibrosing agent induces or stimulates a fibrotic response between the suture and the tissue in contact with the suture.

In another aspect, the present invention provides a method for closing a wound comprising: infiltrating a wound with a fibrosing agent or a composition comprising a fibrosing agent, and joining two surfaces or edges of the wound together with a suture that comprises a fibrosing agent, wherein the fibrosing agent induces or stimulates a fibrotic response between the suture and the tissue in contact with the suture.

In another aspect, the present invention provides a method for performing the Nissen fundoplication procedure, comprising joining portions of the stomach together using a suture that comprises a fibrosing agent.

In another aspect, the present invention provides a method for connecting severed ends of a tendon, comprising joining the severed ends of the tendon together using a suture that comprises a fibrosing agent.

In another aspect, the present invention provides a method for attaching a tendon to a bone, comprising: inserting into the bone a suture anchor that comprises an anchor member and one or more sutures attached to the anchor member, and connecting the end of the tendon to the bone using the one or more sutures, wherein the one or more sutures comprise a fibrosing agent.

In another aspect, the present invention provides a method for attaching a ligament to a bone, comprising: inserting into the bone a suture anchor that has an anchor member and one or more sutures attached to the anchor member, and connecting the end of the ligament to the bone using the one or more sutures, wherein the one or more sutures comprise a fibrosing agent.

In another aspect, the present invention provides a method for laparoscopic insertion of a suture into an abdominal cavity, comprising: placing the suture in a laparoscopic insertion device; inserting the insertion device having the suture through an abdominal wall into the abdominal cavity; and withdrawing the insertion device; wherein the suture comprises a fibrosing agent.

In another aspect, the present invention provides a method for stabilizing an internal structure within the body, comprising: placing a suture into a laparoscopic insertion device; inserting the insertion device with the suture through an abdominal wall; further inserting the insertion device with the suture into the internal structure in need of stabilization; and withdrawing the insertion device; wherein an end portion of the suture remains attached to the internal structure requiring stabilization and another end portion of the suture remains attached to the abdominal wall, thus stabilizing the internal structure, and wherein the suture comprises a fibrosing agent.

In another aspect, the present invention provides a method for forming an anastomosis of a liver bile duct to an intestine, comprising joining an end of the bile duct to an incision in the intestine using a suture that comprises a fibrosing agent.

In another aspect, the present invention provides a method for closing a cystostomy incision, comprising joining two surfaces of the cystostomy incision using a suture that comprises a fibrosing agent.

In another aspect, the present invention provides a method for tying off an appendiceal stump following an appendectomy, comprising: placing a suture around a base of an appendix prior to excising the appendix; excising the appendix, and drawing the suture tight; wherein the suture comprises a fibrosing agent.

In another aspect, the present invention provides a method for repairing a Zenker's Diverticulum, comprising: placing a suture into an endoscopic insertion device; orally inserting the device through two sides of an orifice formed by the Diverticulum; withdrawing the device, leaving the suture in place; using the suture to draw the two sides of the orifice together; and excising the Diverticulum; wherein the suture comprises a fibrosing agent.

In another aspect, the present invention provides a method for repairing a lesion on the interior surface of a viscus structure, comprising joining together the two sides of the lesion using a suture that comprises a fibrosing agent.

In another aspect, the present invention provides a method for attaching a foreign element to a surrounding body tissue, comprising joining a periphery of the foreign element to the tissue using a suture that comprises a fibrosing agent.

In another aspect, the present invention provides a method for mounting a device to a bodily tissue, comprising securing a device to a bodily tissue using a suture that comprises a fibrosing agent.

In another aspect, the present invention provides a method for repositioning tissue, comprising: implanting a tissue connector into tissue in need thereof, and moving the tissue connector so the tissue into which the tissue connector is implanted is repositioned, wherein the tissue connector comprises a fibrosing agent.

In another aspect, the present invention provides a method for closing a wound, comprising joining two surfaces or edges of the wound together with a tissue connector that comprises a fibrosing agent.

In another aspect, the present invention provides a method for performing a skin graft comprising using a tissue connector to hold portions of the graft to underlying tissue wherein the tissue connector comprises a fibrosing agent.

In another aspect, the present invention provides a method for fastening an endoluminal organ or a portion thereof, comprising tying an endoluminal organ or a portion thereof with a self-retaining suture that comprises a fibrosing agent.

These and other aspects of the present invention will become evident upon reference to the following detailed description and attached drawings. In addition, various references are set forth herein which describe in more detail certain procedures and/or compositions (e.g., polymers), and are therefore incorporated by reference in the entirety.

### BRIEF DESCRIPTION OF THE SEVERAL VIEWS OF THE DRAWINGS

FIG. 1 is a bar graph showing the area of granulation tissue in carotid arteries exposed to silk-coated perivascular polyurethane (PU) films relative to arteries exposed to uncoated PU films.

FIG. 2 is a bar graph showing the area of granulation tissue in carotid arteries exposed to silk suture coated perivascular PU films relative to arteries exposed to uncoated PU films.

FIG. 3 is a bar graph showing the area of granulation tissue in carotid arteries exposed to natural and purified silk powder and wrapped with perivascular PU film relative to a control group in which arteries are wrapped with perivascular PU film only.

FIG. 4 is a bar graph showing the area of granulation tissue (at 1 month and 3 months) in carotid arteries sprinkled with talcum powder and wrapped with perivascular PU film relative to a control group in which arteries are wrapped with perivascular PU film only.

FIG. 5 is a bar graph showing indicating the area of perivascular granulation tissue quantified by computer-assisted morphometric analysis in rat carotid arteries treated with control uncoated PU films and with PU films treated with degummed and virgin silk strands. As shown in the figure, both types of silk markedly increased granulation tissue growth around the blood vessel to the same extent.

FIG. 6 shows representative histology sections of rat carotid arteries treated with PU films coated with degummed and virgin silk strands. As shown in the figure, both types of silk induced a marked tissue reaction around the treated blood vessel. Movat stain, 100×.

FIG. 7 shows representative histology sections of rat carotid arteries treated with PU films coated with degummed and virgin silk strands showing the granulation tissue that had grown around the treated vessels. The silk strands had broken down into small particles surrounded by giant cells and macrophages. The granulation tissue was highly vascularized and contained numerous inflammatory cells and fibroblasts. Extracellular matrix deposition was also extensive. H&E stain 200×.

FIG. 8 is a graph showing the effect of cyclosporine A on proliferation of human smooth muscle cells.

FIG. 9 is a graph showing the effect of dexamethasone on proliferation of human fibroblasts.

FIG. 10 is a graph showing the effect of all-trans retinoic acid (ATRA) on proliferation of human smooth muscle cells.

FIG. 11 is a graph showing the effect of isotretinoin on proliferation of human smooth muscle cells.

FIG. 12 is a graph showing the effect of 17-β-estradiol on proliferation of human fibroblasts.

FIG. 13 is a graph showing the effect of 1α, 25-dihydroxy-vitamin D₃ on proliferation of human smooth muscle cells.

FIG. 14 is a graph showing the effect of PDGF-BB on smooth muscle cell migration.

### DETAILED DESCRIPTION OF THE INVENTION

In the following description, certain specific details are included to provide a thorough understanding of various disclosed embodiments. One skilled in the relevant art, however, will recognize that embodiments may be practiced without one or more of these specific details, or with other methods, components, materials, etc.

Sutures have long been the material of choice for deep and superficial wound closure resulting from trauma or as part of surgical procedures. Traditionally, a suture is inserted via a needle on either side of the wound such that it forms an open loop that spans across the tissues to be approximated. The wound is manually pulled together by applying tension on the suture such that the adjacent tissues are physically brought together; their position is finalized when the physician ties a knot to complete the suture loop and affix the adjacent tissues in place. A large number of nondegradable and degradable sutures in numerous lengths, diameters, compositions, needle configurations and filament types have been prepared to accomplish this purpose. However, it has long been recognized that the integrity of the suture material and the knot are essential to successful tissue approximation and wound healing. If the tissue slips over the suture, if the suture material stretches or breaks, or if the knot loosens or becomes untied, tension is lost and the approximated tissues can separate. This can lead to a variety of clinical complications such as wound dehiscence, excessive inflammatory response, procedural failure (particularly devastating when the suture is holding vital structures together), infection, excessive scarring or adhesions, and poor cosmetic results for superficial wounds. In an attempt to reduce this risk, efforts have been made to overcome these deficiencies with “knotless” sutures that can anchor in place without the requirement of knot tying.

To date, self-adhering sutures have been produced in a variety of designs and configurations. The majority of designs centers on the concept of a “barb” structure that eminates from the suture and can be uni-directional or bi-directional in nature. The tissue through which the suture passes becomes “hooked” on the retainers and anchored to the suture material thus preventing the tissue from slipping relative to the suture and (in some cases) providing enough support that knot tying is not required. An additional use for these sutures has been tissue repositioning surgery. Here the self-retaining suture is placed between the supporting tissue and the subcutaneous tissue such that the superficial tissue can be elevated over the barbs, adjusted to the desired anatomical location and affixed in place. The result is repositioning of the superficial tissues over the deeper tissues into a more vertical and youthful position. This largely cosmetic procedure can be applied to virtually any body site, but is most commonly used for facelifts, brow lifts, chin lifts, neck lifts, breast lifts, buttocks lifts and any other location where there are areas of drooping superficial tissues.

Despite this progress, the self-retaining sutures are not without their limitations. Often their hold is insufficient and tissue slippage occurs and impairs wound healing. In some cases, the tissue never becomes completely adherent to the suture or to the adjacent tissues leading to a gradual loss of efficacy. For tissue repositioning procedures, having the elevated tissue permanently adhere to the underlying tissue against which it is placed will not only enhance the durability of the procedure (typically the procedure is only effective for 3-5 years), but will allow the use of fully degradable sutures, since ongoing physical tissue support would not be required.

The present invention provides sutures and self-retaining sutures that comprise a fibrosing agent, as well as methods for making and using such sutures. In addition, the present invention also provides compositions that comprise fibrosing agents and methods for using such compositions in combination with sutures in various applications (e.g., tissue reposition and wound closure). The presence of fibrosing agents in the sutures induces or stimulates a fibrotic response between the sutures and the tissue into which the sutures are inserted, and consequently improves the effectiveness of the sutures in their clinical applications.

### DEFINITIONS

Prior to setting forth the detailed description, it may be helpful to an understanding thereof to first set forth definitions of certain terms that are used hereinafter.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” or “another embodiment” means that a particular referent feature, structure, or characteristic described in connection with the embodiment is included in at least one embodiment. Thus, the appearances of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise.

“Fibrosis,” “Scarring,” or “Fibrotic Response” refers to the formation of fibrous or scar tissue in response to injury or medical intervention.

Therapeutic agents which induce, stimulate, or promote fibrosis or scarring are referred to herein as “fibrosing agents, “scarring agents,” “adhesion-inducing agent,” “fibrosing agent,” and the like, where these agents do so through one or more mechanisms including: inducing or promoting angiogenesis, stimulating migration or proliferation of connective tissue cells (such as fibroblasts, smooth muscle cells, vascular smooth muscle cells), inducing extracellular matrix (ECM) production, and/or promoting tissue remodeling. In the present invention fibrosis will typically be promoted between healing edges of a wound (thereby increasing wound strength) or promote the attachment of superficial tissue to deeper tissues during tissue repositioning surgery. In addition, numerous therapeutic agents described in this invention will have the additional benefit of also promoting tissue regeneration (the replacement of injured cells by cells of the same type).

A therapeutic agent that “induces a fibrotic response” refers to the induction of a fibrotic response in a tissue when the threapuetic agent is present. Such fibrotic response would not occur to the same extent in the absence of the therapeutic agent.

A therapeutic agent that “stimulates a fibrotic response” refers to the increase in the degree of a fibrotic response in a tissue when the therapeutic agent is present compared with that when the therapeutic agent is absent.

“Suture” refers to the fine thread or other material used to close a wound, join tissues, or perform repositioning procedures. Sutures include both plain sutures and self-retaining sutures, and may comprise bioabsorbable or nonabsorbable material.

“Plain suture” refers to a suture without any barbsor other retainers located along the body of the suture.

“Self-retaining suture” refers to a suture with one or more retainers located along the suture. The retainers are of sufficient size and appropriate geometry for fastening to, or gripping, the tissue through which the self-retaining suture is inserted and achieving closure of an incision or wound (or repositioning tissue) with superior attachment or without the need for tying knots. Retainers may be configured to have tissue insertion points (such as barbs), tissue insertion edges (such as conical or frusto-conical retainers), and so forth.

“One-directional self-retaining suture” (also referred to as “one-directional suture,” “one-way self-retaining suture,” “one-way suture,” “uni-directional self-retaining suture,” or “uni-directional suture”) refers to a suture having retainers on its exterior surface and facing towards one end of the suture. Such arrangement of retainers on the suture allows the suture to be drawn in only one direction through tissue, but not in the opposite direction.

“Two-way self-retaining suture” (also referred to “two-way suture,” “two-directional self-retaining suture,” “two-directional suture,” “bi-directional self-retaining suture,” or “bi-directional suture”) refers to a suture that has retainers facing toward one end of the suture for about half the suture length and retainers facing the opposite direction toward the other end of the suture for the other half of the suture length. This arrangement allows the retainers to move in the same direction as each respective suture end is inserted into host tissue.

“Wound”, as used herein, refers to an opening of the skin or other bodily tissue resulting from an injury, surgical procedure, or a disease. Wounds include incisions, cuts, lacerations, punctures, and penetrations. It may also include chronic wounds such as arterial ulcers, fistulas, sinus tracts, abscesses, venous ulcers, lymphedema, pressure ulcers, and neuropathic ulcers. A wound in a tissue can be of any configuration and in any anatomical part or organ of the body. Accordingly, a wound may comprise several sides and faces and is not limited to a straight incision.

“Repositioning a tissue” refers to moving a tissue from its original position to a different anatomical position.

“Localized delivery” refers to administration of a therapeutic agent from a device, implant (e.g., a suture) or composition into or near a tissue in need of the therapeutic agent and provides a high local (regional) concentration of the therapeutic agent at or near the site of suture implantation. In certain aspects, the fibrosing agent or composition that comprises the fibrosing agent is released from an implant (e.g., a suture) locally into or in the vicinity of the site where the suture is implanted. In other aspects, “localized delivery” is achieved by direct contact between the surface of a suture and the surface of the tissue in contact with the suture.

“Release of an agent from a suture” refers to any statistically significant dissociation of an agent, or a subcomponent thereof from a suture.

“Biodegradable” (used interchangeably with “degradable” or “absorbable”) refers to materials for which the degradation process is at least partially mediated by, or performed in, a biological system. “Degradation” refers to a chain scission process by which a polymer chain is cleaved into oligomers and monomers. Chain scission may occur through various mechanisms, including, for example, by chemical reaction (e.g., hydrolysis, oxidation/reduction, enzymatic mechanisms or a combination or these) or by a thermal or photolytic process. Polymer degradation may be characterized, for example, using gel permeation chromatography (GPC), which monitors the polymer molecular mass changes during erosion and drug release. “Biodegradable” also refers to materials may be degraded by an erosion process at least partially mediated by, or performed in, a biological system. “Erosion” refers to a process in which material is lost from the bulk. In the case of a polymeric system, the material may be a monomer, an oligomer, a part of a polymer backbone, and/or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage, enzymatic cleavage or a combination of these; (2) polymers that are initially water insoluble are solubilized by hydrolysis, enzymatic cleavage, ionization, or pronation of a pendant group or a combination of these mechanisms; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance (EPR) spectroscopy, NMR imaging, and recording mass loss during an erosion experiment. For microspheres, photon correlation spectroscopy (PCS) and other particles size measurement techniques may be applied to monitor the size evolution of erodible devices versus time.

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. As used herein, the term “about” means±15%.

### A. Fibrosing Agents

Numerous fibrosing agents have been identified that can be used in combination with a suture according to the present invention. The agents may be further formulated with one or more other materials, such as another therapeutic agent (e.g., an anti-infective agent, anti-proliferative agent or an anti-inflammatory agent) and/or a polymeric carrier, which formulations are discussed below. Many suitable fibrosing agents are specifically identified herein, and others may be readily determined based upon in vitro and in vivo (animal) models such as those provided in the examples. Fibrosing agents can be identified through in vivo models such as the rat carotid artery model.

In one aspect, a fibrosing agent may be a tissue irritant. A tissue irritant may be any material that causes a localized inflammatory response.

Tissue irritants may be inorganic materials (e.g., metals, minerals, or ceramics) such as talcum powder, metallic beryllium and oxides thereof, copper, silica, crystalline silicates, talc, or quartz dust, and inflammatory microcrystals (e.g., crystalline minerals such as crystalline silicates).

Other examples of tissue irritants include synthetic organic compounds such as ethanol, and carbon tetrachloride.

In certain aspects, the fibrosing agent may be a sclerosing agent. “Sclerosing” refers to a tissue reaction in which a compound (e.g., an irritant) is applied locally to a tissue which results in an inflammatory reaction and is followed by scar tissue formation at the site of irritation. A pharmaceutical agent that induces or promotes sclerosis is referred to as a “sclerosant,” or a “sclerosing agent.” Representative examples of sclerosants include ethanol, dimethyl sulfoxide, surfactants (e.g., TRITON X, sorbitan monolaurate, sorbitan sesquioleate, glycerol monostearate and polyoxyethylene, polyoxyethylene cetyl ether, etc.), sucrose, sodium chloride, dextrose, glycerin, minocycline, tetracycline, doxycycline, polidocanol, sodium tetradecyl sulfate, sodium morrhuate, ethanolamine, phenol, sarapin and sotradecol. In certain aspects, any of the tissue irritants described herein may function as sclerosing agents depending on the type and amount of compound that contacts the tissue.

Fibrosing agents include a variety of naturally occurring and synthetic polymers.

In one aspect, the fibrosing agent may be a naturally occurring polymer such as a protein (e.g., silk, fibroin, sericin, animal wool, or collagen).

In one embodiment, the fibrosing agent suitable for use in combination with a suture is silk. Silk refers to a fibrous protein, and may be obtained from a number of sources, typically spiders and silkworms. Typical silks contain about 75% of actual fiber, referred to as fibroin, and about 35% sericin, which is a gummy protein that holds the filaments together. Silk filaments are generally very fine and long—as much as 300-900 meters long. There are several species of domesticated silkworm that are used in commercial silk production, however, Bombyx mori is the most common, and most silk comes from this source. Other suitable silkworms include Philosamia cynthia ricini, Antheraea yamamai, Antheraea pernyi, and Antheraea mylitta. Spider silk is relatively more difficult to obtain, however, recombinant techniques hold promise as a means to obtain spider silk at economical prices (see, e.g., U.S. Pat. Nos. 6,268,169; 5,994,099; 5,989,894; and 5,728,810, which are exemplary only). Biotechnology has allowed researchers to develop other sources for silk production, including animals (e.g., goats) and vegetables (e.g., potatoes). Silk from any of these sources may be used in the present invention.

Silk may take a variety of forms and may be in a raw or a processed state. For example, silk may be virgin (raw) silk or degummed silk and may take the form of a suture (e.g. coated or uncoated silk sutures), filaments or threads, or particles.

A commercially available silk protein is available from Croda, Inc., of Parsippany, N.J., and is sold under the trade names CROSILK LIQUID (silk amino acids), CROSILK 10,000 (hydrolyzed silk), CROSILK POWDER (powdered silk), and CROSILKQUAT (cocodiammonium hydroxypropyl silk amino acid). Another example of a commercially available silk protein is SERICIN, available from Pentapharm, LTD, a division of Kordia, BV, of the Netherlands. Further details of such silk protein mixtures can be found in U.S. Pat. No. 4,906,460, to Kim, et al., assigned to Sorenco. Silk useful in the present invention includes natural (raw) silk, hydrolyzed silk, and modified silk, i.e., silk that has undergone a chemical, mechanical, or vapor treatment, e.g., acid treatment or acylation (see, e.g., U.S. Pat. No. 5,747,015).

Raw silk is typically twisted into a strand sufficiently strong for weaving or knitting. Four different types of silk thread may be produced by this procedure: organzine, crepe, tram and thrown singles. Organzine is a thread made by giving the raw silk a preliminary twist in one direction and then twisting two of these threads together in the opposite direction. Crepe is similar to organzine but is twisted to a much greater extent. Twisting in only one direction two or more raw silk threads makes tram. Thrown singles are individual raw silk threads that are twisted in only one direction. Any of these types of silk threads may be used in the present invention.

The silk used in the present invention may be in any suitable form that allows the silk to be joined with a suture, for example, the silk may be in a powder-based form. The silk can be prepared in the powdered form by several different methods. For example the silk can be milled (e.g., cryomill) into a powdered form. Alternatively the silk can be dissolved in a suitable solvent (e.g., HFIP or 9M LiBr) and then sprayed (electrospray, spray dry) or added to a non-solvent to produce a powder. Furthermore, the silk may have any molecular weight, where various molecular weights are typically obtained by the hydrolysis of natural silk, where the extent and harshness of the hydrolysis conditions determines the product molecular weight. For example, the silk may have an average (number or weight) molecular weight of about 200 to 5,000. See, e.g., JP-B-59-29199 (examined Japanese patent publication) for a description of conditions that may be used to hydrolyze silk.

A discussion of silk may be found in the following documents, which are exemplary only, Hinman, M. B., et al. “Synthetic spider silk: a modular fibre” Trends in Biotechnology, 2000, 18(9) 374-379; Vollrath, F. and Knight, D.P. “Liquid crystalline spinning of spider silk” Nature, 2001, 410(6828) 541-548; and Hayashi, C. Y., et al. “Hypotheses that correlate the sequence, structure, and mechanical properties of spider silk proteins” Int. J. Biol. Macromolecules, 1999, 24(2-3), 265-270; and U.S. Pat. No. 6,427,933.

The silk may be virgin silk, partially degummed, or degummed silk. The silk can also further comprise a coating. The coating may be a silicone-based coating, a wax based coating, or a degradable polymer based coating.

In another embodiment, the fibrosing agent is a fibroin protein, or a fragment or fragments thereof. In a certain embodiment, the fibroin protein may be a synthetic analogue that is made and that has one or more of the known repeat sequences of the fibroin protein.

In another embodiment, the fibrosing agent is sericin. Sericin is a component of virgin silk that can be used to assist in the induction of a fibrotic response.

Another exemplary fibrosing agent is wool. The term “wool” refers to an entangled mass of fibers without any ordered arrangement, while the term “fiber” refers to a particle with a length to diameter ratio (“aspect ratio”) of at least about 3:1 and roughly parallel edges.

Wool that may be used in the compositions and methods described herein induces an enhanced fibrotic response between the suture and the tissue adjacent to the suture. In other words, absent the wool, the suture would generate a “normal” adhesion between the adjacent tissue and the suture, while in the presence of the wool, the same suture is capable of generating an enhanced adhesion (e.g., via an enhanced matrix deposition response to the presence of the wool).

Wool useful as a fibrosing agent may be obtained or prepared from natural sources (e.g., animal wool and wood wool). Alternatively, it may be artificially synthesized (e.g., polymeric wool and mineral wool).

“Animal wool” refers to animal hair fibers, typically derived from the fleece of sheep or lamb, goat (e.g., Angora and Cashmere), camel, alpaca, llama, vicuna, or the like. Animal wool is a dead tissue that has a complex morphological and chemical structure, which make it unique among textile fibers. Morphologically, wool fibers are biological composites, with each component having a different physical and chemical composition. Wool fibers are generally composed of three different types of spindle-shaped cortical cells surrounded by a sheath of overlapping, rectangular cell known as the cuticle, which forms the external layer of the fiber. Approximately 90% of the cortical cell type is made up of longitudinally arrayed intermediate filaments with accompanying matrix. The remainder includes membranes and remnants from the nucleus and cytoplasm.

Animal wool fibers exhibit a range of diameters, lengths, and crimp (i.e., a measure of fiber curvature), which allows the wool fibers to entrap air. Animal wool is also hygroscopic and is able to absorb and desorb large amounts of water as the relative humidity surrounding the fiber changes. Furthermore, animal wool liberates heat if it absorbs water. These properties contribute to animal wool's extraordinary insulating quality.

Animal wool belongs to a family of proteins called a-keratins, which also include materials such as hooves, horns, nails, claws, and beak. A characteristic feature of a-keratins (also referred to as “hard” keratins) is a higher concentration of sulfur than “soft” keratins, such as those in the skin. Clean animal wool contains about 82% keratinous proteins that are high in sulfur content, and about 17% of the fiber is protein with a relatively low sulfur content (<3%). The sulfur in wool occurs in the form of the amino acid cysteine. Due to the high cysteine content, animal wool is highly cross-linked by disulfide bonds that render it essentially insoluble. It is estimated that animal wool contains about 170 different types of polypeptides varying in relative molecular mass from below 10,000 to greater than 50,000. The groups of proteins that constitute animal wool are not uniformly distributed throughout the fiber but are aggregated within various regions. Animal wool also contains about 1% non-proteinaceous material that consists mainly of free and structural lipids and polysaccharide materials, trace elements, and pigments (e.g., melanin).

Animal wool is usually harvested from animals by annual shearing. Thus, the fiber length is determined largely by the rate of growth, which in turn depends on both genetic and environmental factors. For instance, typical merino fibers are 50-125 mm long and have irregular crimp (curvature). Animal wool fibers exhibit a range of diameters, which also depend on both genetics and environment. For example, coarse wool fibers generally have a diameter of 25-70 mm, while fine merino fibers typically have a diameter of 10-25 mm.

Another example of a naturally derived wool is wood wool, which is a specially prepared, non-compressed wood fiber frequently used in surgical dressings and packaging materials. Wood wool fibers also can be obtained from pine needles.

Although wool is usually associated with fibers derived from natural sources, a variety of synthetic wool is also available. Synthetic wool includes, for example, mineral wools, such as glass wool, stone wool, and slag wool, and wool made from polymeric materials. Mineral wool may be formed, for example, from a molten, inorganic material such as glass, stone, or slag that is spun into a fiber-like structure. Inorganic rock or slag is the main component (typically 98%) of stone wool. The remaining 2% organic content is generally a thermosetting resin binder (an adhesive) and a small amount of oil. Glass wool products usually contain about 95% inorganic material. Glass wool is made from sand or recycled glass, limestone, and soda ash, which are the same ingredients used for familiar glass objects such as window panes or glass bottles. Glass fiber may, additionally, include a small amount of boron. Stone wool can be made from volcanic rock, typically basalt or dolomite. Slag wool is made from blast furnace slag (waste).

A discussion of wool may be found in the following documents, which are exemplary only: Encyclopedia of Polymer Science and Technology, John Wiley & Sons, Inc. (2003); Dowling and Sparrow, TIBS16:115-118 (1991); Powman, J. Chromatogr. B 787:63-76 (2003); Hearle, Intl. J. Biol. Macromol. 27:123-38 (2000); and Vuyst et al., Eur. Resp. J. 8:2149-73 (1995).

In certain embodiments, wool fibers have an average length of about, or at least about, 1, 2, 3, 4, 5, 6, 7, 8, 9, 10, 15, 20, 25, 30, 35, 40, 45, 50 mm or longer. In certain embodiments, the lengths of wool fibers are in a range of about 1-5 mm, 5-10 mm, 10-50 mm, 50-100 mm, 1-10 mm, 1-50 mm, or 1-100 mm. In certain embodiments, wool fibers have an average diameter of about, or at least about, 1, 2, 3, 4, 5, 6, 7, 8, 9, 10, 15, 20, 25, 30, 35, 40, 45, or 50 mm. In certain embodiments, the diameters of wool fibers are in a range of about 1-3 mm, 3-5 mm, 5-10 mm, 10-50 mm, 1-10 mm, or 1-50 mm. In certain embodiments, the average length to diameter ratio of wool fibers is 3:1, 4:1, 5:1, 6:1, 7:1, 8:1, 9:1, 10:1 or larger.

Wool may be used alone or may be used in combination with a suture, such as described herein. In certain embodiments, wool may be used in combination with one or more of other fibrosing agents described herein.

Other fibrosing agents that promote scar formation include components of extracellular matrix (ECM) and proteins that bind to ECM (e.g., fibronectin, fibrillin, fibrin, fibrinogen, collagen (e.g., bovine collagen), fibrillar and non-fibrillar collagen, vitronectin, fibronectin, laminin, hyaluronic acid, elastin, adhesive glycoproteins, proteoglycans (e.g., heparin sulfate, chondroitin sulfate, dermatan sulfate), hyaluronan, secreted protein acidic and rich in cysteine (SPARC), thrombospondins, tenacin, cell adhesion molecules (including integrins, selectins, ICAMs and HA-binding proteins such as CD44), proteins found in basement membranes, and fibrosin.

Fibrosing agents may be growth factors or inflammatory cytokines involved in angiogenesis, fibroblast migration, fibroblast proliferation, ECM synthesis and tissue remodeling, such as epidermal growth factor (EGF) family, transforming growth factor-α (TGF-α), transforming growth factor-β (TGF-β-1, TGF-β-2, TGF-β-3, platelet-derived growth factor (PDGF), fibroblast growth factor (acidic—aFGF; and basic—bFGF), fibroblast stimulating factor-1, activins, vascular endothelial growth factor (including VEGF-2, VEGF-3, VEGF-A, VEGF-B, VEGF-C, placental growth factor—PIGF), angiopoietins, insulin-like growth factors (IGF) such as IGF-a, hepatocyte growth factor (HGF), connective tissue growth factor (CTGF), myeloid colony-stimulating factors (CSFs), monocyte chemotactic protein, granulocyte-macrophage colony-stimulating factors (GM-CSF), granulocyte colony-stimulating factor (G-CSF), macrophage colony-stimulating factor (M-CSF), erythropoietin, interleukins (particularly IL-1, IL-8, and IL-6), tumor necrosis factor-α (TNFα), nerve growth factor (NGF), interferon-α, interferon-β, histamine, endothelin-1, angiotensin II, growth hormone (GH), and synthetic peptides, IL-1, IL-3, IL-11, IL-13, IL-15, IL-18, IL-23, macrophage inflammatory protein-1γ (MIP-1γ), CD40 and CD40 ligand, arginase (ARG-1) agonists such as L-arginine and L-ornithine, TNFα inhibitors such as infliximab, etanercept, adalimumab, and analogues or derivatives of these.

The fibrosing agent may be a bone morphogenic protein (BMP) (e.g., BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Of these, BMP-2, BMP-3, BMP-4, BMP-5, BMP-6, and BMP-7 are of particular utility. Bone morphogenic proteins are described, for example, in U.S. Pat. Nos. 4,877,864; 5,013,649; 5,661,007; 5,688,678; 6,177,406; 6,432,919; and 6,534,268 and Wozney, J. M., et al. (1988) Science: 242(4885); 1528-1534.

The fibrosing agent may be naturally occurring or synthetic polypeptide (e.g., peptides with high lysine content such as polylysine or naturally occurring or synthetic peptides containing the Arg-Gly-Asp (RGD) sequence, generally at one or both termini (see, e.g., U.S. Pat. No. 5,997,895)).

The fibrosing agent may be a polysaccharide such as chitosan or N-carboxybutylchitosan.

The fibrosing agent may be a cellulosic material, such as cotton.

Fibrosing agents may be synthetic polymers (e.g., polyurethanes, poly(ethylene terephthalate) (e.g., DACRON), poly(ethylene-co-vinylacetate), polymers of vinyl chloride, polytetrafluoroethylene (PTFE), or cyanoacrylates, such as poly(alkylcyanoacrylates)).

Fibrosing agents may be tissue adhesives, such as cyanoacrylates, fibrin glues, or crosslinked poly(ethylene glycol) and crosslinked poly(ethylene glycol)-methylated collagen compositions, such as are described in published U.S. Patent Application No. 200510148512A1, incorporated herein by reference in its entirety.

In certain embodiments, the fibrosing agent may be a hemostat, which is capable of promoting fibrosis. Exemplary compositions that can function as hemostats are crosslinked compositions that comprise thiol- or amino-functional groups. For example, amino-functionalized polyethylene glycol (e.g., 4-armed tetra-amino PEG [10k]) can be reacted with a 4-armed NHS functionalized PEG (e.g., pentaerythritol poly(ethylene glycol)ether tetra-succinimidyl glutarate) under basic buffer conditions. In another example, a 4-armed thiol functionalized PEG (e.g., pentaerythritol poly(ethylene glycol)ether tetra-thiol) can be substituted for the 4-arm amino-functionalized PEG such that the amount of amino functional groups in the final composition can be varied. These reagents can be mixed at the time of application to provide an in situ forming crosslinked hydrogel. These reagents could be premixed to produce the crosslinked material. The material can be made in various forms such as rods, tubes, films, meshes, screens, threads, fibers, slabs, or spheres. The crosslinked material could also be milled to produce a particulate material. These materials can be dried (e.g., air, vacuum, freeze-dried) and used as a dry powdered material. Alternatively the materials can be hydrated just prior to application. These materials can further comprise one of the fibrosing agents described herein.

Other examples of fibrosing agents include bleomycin or an analogue or derivative thereof, methotrexate, bromocriptine, methylsergide, thioacetamide, or fibrosin.

In one aspect, the fibrosing agent may be a proliferative agent that stimulate cellular proliferation, for example, dexamethasone, isotretinoin, 17-β-estradiol, estradiol, diethylstibesterol, cyclosporine A, all-trans retinoic acid (ATRA), estradiol 17-valerate, β-estradiol 17-cypionate, β-estradiol 17-propionate, 2-methoxy-17-beta-estradiol, and analogues and derivatives thereof.

In certain embodiments, two or more fibrosing agents may be used in combination with sutures according to the present invention. The effectiveness of various combinations of fibrosing agents in using in combination with sutures (e.g., closing wound) may be determined according to the methods described in the examples.

### B. Therapeutic Compositions

Fibrosing agents as described above may be used in combination with other therapeutic agents or components to form therapeutic compositions. In certain embodiments, such compositions may be used in making sutures that comprise fibrosing agents, such as used as a coating or dipping solution. In other embodiments, fibrosing agent-containing therapeutic compositions may be used to infiltrate tissue into which a suture (e.g., a self-retaining suture) has been, is being, or is to be implanted.

1. Secondary Therapeutic Agents

Within various embodiments of the invention, a therapeutic composition may include an agent that promotes fibrosis and a second composition or compound which acts to have an inhibitory effect on pathological processes in or around the site where a suture has been, is being, or is to be implanted.

The secondary agent may be an: anti-inflammatory agent (e.g., dexamethasone, cortisone, fludrocortisone, prednisone, prednisolone, 6α-methylprednisolone, triamcinolone, betamethasone, or amcinonide).

The secondary agent may be a matrix metalloproteinase (MMP) inhibitor (e.g., marimistat, batimistat, or TIMP's, representative examples of which are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359). Other examples of MMP inhibitors include nimesulide, PKF-241-466, PKF-242-484, CGS-27023A, SAR-943, primomastat, SC-77964, PNU-171829, AG-3433, PNU-142769, SU-5402, and dexlipotam.

The secondary agent may be a cytokine inhibitor (e.g., chlorpromazine, mycophenolic acid, rapamycin, 1α-hydroxy vitamin D₃).

The secondary agent may be an inosine monophosplate dehydrogenase inhibitor (IMPDH) (e.g., mycophenolic acid, ribaviran, aminothiadiazole, thiophenfurin, tiazofurin, or viramidine). Representative examples are described in U.S. Pat. Nos. 5,536,747; 5,807,876; 5,932,600; 6,054,472; 6,128,582; 6,344,465; 6,395,763; 6,399,773; 6,420,403; 6,479,628; 6,498,178; 6,514,979; 6,518,291; 6,541,496; 6,596,747; 6,617,323; and 6,624,184, U.S. Patent Application Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491 A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201 A1, 2003/0181497A1, 2003/0186974A1, 2003/0186989A1, and 2003/0195202A1, and PCT Publication Nos. WO 00/24725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 02/051814A1, WO 02/057287A2, WO 02/057425A2, WO 02/060875A1, WO 02/060896A1, WO 02/060898A1, WO 02/068058A2, WO 03/020298A1, WO 03/037349A1, WO 03/039548A1, WO 03/045901 A2, WO 03/047512A2, WO 03/053958A1, WO 03/055447A2, WO 03/059269A2, WO 03/063573A2, WO 03/087071 A1, WO 99/001545A1, WO 97/40028A1, WO 97/41211A1, WO 98/40381A1, and WO 99/55663A1.

The secondary agent may be a p38 MAP kinase inhibitor (MAPK) (e.g., GW-2286, CGP-52411, BIRB-798, SB220025, RO-320-1195, RWJ-67657, RWJ-68354, SCIO-469, CGH-2466 and PD-98-59). Representative examples are included in U.S. Pat. Nos. 6,300,347; 6,316,464; 6,316,466; 6,376,527; 6,444,696; 6,479,507; 6,509,361; 6,579,874, and 6,630,485, and U.S. Patent Application Publication Nos. 2001/0044538A1, 2002/0013354A1, 2002/0049220A1, 2002/0103245A1, 2002/0151491A1, 2002/0156114A1, 2003/0018051A1, 2003/0073832A1, 2003/0130257A1, 2003/0130273A1, 2003/0130319A1, 2003/0139388A1, 2003/0139462A1, 2003/0149031A1, 2003/0166647A1, and 2003/0181411A1, and PCT Publication Nos. WO 00/63204A2, WO 01/21591A1, WO 01/35959A1, WO 01/74811A2, WO 02/18379A2, WO 02/064594A2, WO 02/083622A2, WO 02/094842A2, WO 02/096426A1, WO 02/101015A2, WO 02/103000A2, WO 03/008413A1, WO 03/016248A2, WO 03/020715A1, WO 03/024899A2, WO 03/031431A1, WO 03/040103A1, WO 03/053940A1, WO 03/053941A2, WO 03/063799A2, WO 03/079986A2, WO 03/080024A2, WO 03/082287A1, WO 97/44467A1, WO 99/01449A1, and WO 99/58523A1.

The secondary agent may be an immunomodulatory agent (e.g., sirolimus (rapamycin) and analogues or derivatives thereof, azathioprine, azithromycin, tacrolimus and derivatives thereof (see, e.g., EP 0184162B1 and those described in U.S. Pat. No. 6,258,823), and everolimus and derivatives thereof (see, e.g., U.S. Pat. No. 5,665,772). Further representative examples of sirolimus analogues and derivatives include ABT-578 and those found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179 and in U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

The immunomodulatory agent may be an immunosuppressant (e.g., argyrin B, ADZ-62-826, CCI-779 (temsirolimus-mTOR inhibitor), tilomisole, FK-778, AVE-1726, MDL-28842, TNF-484A, CP-293121, CP-353164, and PD-168787).

The secondary agent may be an anti-helminthic, such as a macrocyclic lactone, e.g. mebendazole, pyrantel pamoate, piperazine citrate, albendazole, and metronidazole.

The secondary agent may be a CCR2/MCP-1 inhibitor, such as PD-172084.

The secondary agent may be a tyrosine kinase inhibitor (e.g., imatinib, ZK-222584, CGP-52411, CGP-53716, NVP-AAK980-NX, CP-127374, CP-564959, PD-171026, PD-173956, PD-180970, SU-0879, and SKI-606).

The secondary agent may be a NFKB inhibitor (e.g., AVE-0547, AVE-0545, and IPL-576092).

The secondary agent may be an HMGCoA reductase inhibitor (e.g., pravastatin, atorvastatin, fluvastatin, dalvastatin, glenvastatin, pitavastatin, CP-83101, U-20685), cerivastatin, rosuvastatin, lovastatin, mevastatin, and simvastatin).

The secondary agent may be an apoptosis antagonist (e.g., troloxamine, TCH-346 (N-methyl-N-propargyl-10-aminomethyl-dibenzo(b,f)oxepin),

The secondary agent may be a caspase inhibitor (e.g., PF-5901 (benzenemethanol, alpha-pentyl-3-(2-quinolinylmethoxy)-).

The secondary agent may be a JNK inhibitor (e.g., AS-602801).

Within various embodiments, the present invention provides a composition that promotes fibrosis and also stimulates cellular proliferation. Some of these agents are known to stimulate proliferation at relatively low concentrations and inhibit proliferation at high concentrations. The concentrations that stimulate proliferation are readily determined through assays that measure an agent's ability to affect the rate of cell proliferation (see examples). Representative examples of agents that stimulate cellular proliferation include pyruvic acid, naltrexone, leptin, D-glucose, insulin, amlodipine, alginate oligosaccharides, minoxidil, dexamethasone, isotretinoin (13-cis retinoic acid), 17-β-estradiol, estradiol, 1-α-25 dihydroxyvitamin D₃, diethylstibesterol, cyclosporine A, L-NAME (L-NG-nitroarginine methyl ester (hydrochloride)), all-trans retinoic acid (ATRA), and analogues and derivatives thereof. Other examples of agents that stimulate cellular proliferation include sphingosine 1-phosphate receptor agonist (e.g., FTY720 (1,3-propanediol, 2-amino-2-(2-(4-octylphenyl)ethyl)-, hydrochloride); immunostimulants, such as Imupedone (methanone, [5-amino-2-(4-methyl-1-piperidinyl)phenyl](4-chlorophenyl)-, DIAPEP227 synthetic peptide (Peptor Ltd., Israel)); and nerve growth factor agonist, e.g., NG-012 (5H,9H,13H,21H,25H,-dibenzo[k,u][1,5,9,15,19] pentaoxacyclotetracosin-5,9,13,21,25-pentone, 7,8,11,12,15,16,23,24,27,28-decahydro-2,4,18,20-tetrahydroxy-11-(hydroxymethyl)-7,15,23,27-tetramethyl-, NG-121, SS-701 (2,2′:6′,2″-terpyridine, 4′-(4-methylphenyl)-, trihydrochloride, AMPAIex (piperidine, 1-(6-quinoxalinylcarbonyl)-, RGH-2716 (8-[4,4-bis(4-fluorophenyl)butyl]-3-(1,1-dimethylethyl)-4-methylene-1-oxa-3,8-diaza-spiro[4.5] decan-2-one, and TDN-345 (1-oxa-3,8-diazaspiro[4.5]decan-2-one, 8-[4,4-bis(4-fluorophenyl)butyl]-3-(1,1-dimethylethyl)-4-methylene-).

Additional examples of compounds which are capable of stimulating cellular processes which result in tissue growth include pyruvic acid, hyaluronic acid, naltrexone, estrogen, leptin, statins, D-glucose, insulin, sphingosine 1-phosphate, amlodipine, alginate oligosaccharides, and minoxidil, including analogues and derivatives of these.

In certain embodiments, the therapeutic composition may comprise one or more anti-infective agents, which may reduce the likelihood of infections at the site where a suture is implanted. An “anti-infective agent” refers to an agent that reduces the likelihood of an infection. An agent is demonstrated to be an active anti-infective agent toward a microorganism by assays routinely practiced by persons skilled in the art, for example, an in vitro assay determining inhibition of bacterial growth as indicated by the M.I.C. (minimimum inhibitory concentration). In certain embodiments, anti-infective agents are chemotherapeutic agents that have antimicrobial activity at low doses (e.g., anthracyclines, fluoropyrimidines, folic acid antagonists, podophylotoxins, camptothecins, hydroxyureas, and platinum complexes.

In certain embodiments, the anti-infective agent may be an anti-septic agent. An “anti-septic agent” refers to an agent or substance that is capable of effective antisepsis, that is, prevention of infection by inhibiting the growth of an infectious organism without necessarily killing the organism. Representative examples of anti-septic agents include chlorhexadine, triclosan, and chloroxylenol.

In certain other embodiments, the anti-infective agent may be an antibiotic. An “antibiotic” refers to an agent that kills or inhibits the growth of microorganisms. Antibiotics may have a narrow or wide range of activity against either one or both of Gram-positive and Gram-negative organisms. Antibiotic agents can be identified through in vitro inhibition of bacterial growth as shown in the M.I.C. assay described herein. Representative examples of antibiotics include gentamicin sulfate, amikacin sulfate, kanamycin sulfate, polymyxin B, neomycin sulfate, cephazolin sodium, metronidazole, Ciprofloxacin, piperacillin, Cefoxitin, Cefepime, Azithromycin, and Trimethoprom-sulfamethoxazole.

a. Anti-Infective Agents—Antibiotics

Antibiotics and combinations of antibiotics that are used by those skilled in the medical art include the following exemplary antibiotics: fourth generation penicillins such as mezlocillin and piperacillin (ureidopenicillins), carbenicillin and ticarcillin (carboxypenicillins), and analogues and derivatives thereof; first generation cephalosporins such as cephazolin, Cephazolin Sodium, Cephalexin (Keflex), Cefazolin (Ancef), Cephapirin (Cefadyl), and Cephalothin (Keflin), and analogues and derivatives thereof; Ticarcillin; second generation cephalosporins such as Cefuroxime (Ceftin (oral) and Zinocef), Cefotetan (Cefotan), and Cefoxitin (Mefoxin), and analogues and derivatives thereof; third generation cephalosporin such as Naxcel (Ceftiofur Sodium), Cefdinir (Omnicef), Cefoperazone (Cefobid), Ceftazidime (Fortaz), and Ceftriaxone (Rocephin), and Cefotaxime (Claforan), and analogues and derivatives thereof; and fourth generation cephalosporins such as Cefepime (Maxipime) and analogues and derivatives thereof; monobactams such as aztreonam and analogues and derivatives thereof; carbapenems such as imipenem, ertapenem and meropenem, and analogues and derivatives thereof. Also included are inhibitors of protein synthesis such as aminoglycosides including streptomycin, gentamicin, gentamicin sulfate, tobramycin, and amikacin, amikacin sulfate, and analogues and derivatives thereof; inhibitors of protein synthesis such as the MSL group including macrolides (Erythromycin), long acting macrolides (Azithromycin) and lincosamides (Clindamycin) and streptogramins (Syneroid), clarithromycin, kanamycin, kanamycin sulfate, and analogues and derivatives thereof. Other exemplary antibiotics include inhibitors of DNA synthesis such as the quinolones including ciprofloxacin, ofloxacin, gatifloxacin, moxifloxacin, levofloxacin, trovafloxacin, and analogues and derivatives thereof, as well as other inhibitors of DNA synthesis such as metronidazole and analogues and derivatives thereof. Other antibiotics include inhibitors of folate metabolism such as sulfonamides and trimethoprim, and analogues and derivatives thereof. Additional agents include but are not limited to cefixime, spectinomycin, tetracycline, nitrofurantoin, doxycycline, polymyxin B, neomycin, neomycin sulfate, and analogues and derivatives thereof. In certain embodiments, the anti-infective agent is gentamicin sulfate, amikacin sulfate, kanamycin sulfate, polymyxin B, neomycin sulfate, cephazolin sodium, metronidazole, ciprofloxacin, piperacillin, cefoxitin, cefepime, azithromycin, or trimethoprim-sulfamethoxazole.

Furthermore, additional therapeutic agents may be delivered in combinations. Such combinations include, by way of example, but are not limited to amoxicillin and clavulanate, ampicillin and sulbactam, trimethoprom-sulfamethoxazole, ampicillin and probenecid, amoxicillin and probenecid, penicillin G and probenecid, and penicillin and a penicillinase inhibitor.

b. Anti-Infective Agents—Chemotherapeutic Agents

In certain embodiments, anti-infective agents useful in the present invention may be chemotherapeutic agnets, which have potent antimicrobial activity at extremely low doses. Discussed in more detail below are several representative examples of such agents: (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin).

i. Anthracyclines

Anthracyclines have the following general structure, where the R groups may be a variety of organic groups:

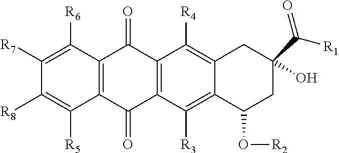

According to U.S. Pat. No. 5,594,158, suitable R groups are as follows: R₁ is CH₃ or CH₂OH; R₂ is daunosamine or H; R₃ and R₄ are independently one of OH, NO₂, NH₂, F, Cl, Br, I, CN, H or groups derived from these; R₅ is hydrogen, hydroxyl, or methoxy; and R₆₋₈ are all hydrogen. Alternatively, R₅ and R₆ are hydrogen and R₇ and R₈ are alkyl or halogen, or vice versa.

According to U.S. Pat. No. 5,843,903, R₁ may be a conjugated peptide. According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure:

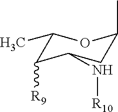

in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a O₃₋₄ membered alkylene with R₁₂. R₁₂ may be H, alkyl, aminoalkyl, amino, hydroxyl, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

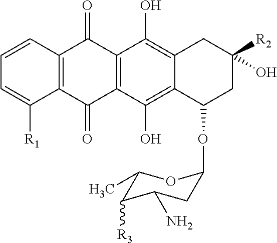

| | R₁ | R₂ | R₃ |
|---|---|---|---|
| Doxorubicin: | OCH₃ | C(O)CH₂OH | OH out of |
| | | | ring plane |
| Epirubicin: | OCH₃ | C(O)CH₂OH | OH in |
| (4′ epimer of | | | ring plane |
| doxorubicin) | | | |
| Daunorubicin: | OCH₃ | C(O)CH₃ | OH out of |
| | | | ring plane |
| Idarubicin: | H | C(O)CH₃ | OH out of |
| | | | ring plane |
| Pirarubicin: | OCH₃ | C(O)CH₂OH | 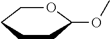 |
| Zorubicin: | OCH₃ | C(CH₃)(═N)NHC(O)C₆H₅ | OH |
| Carubicin: | OH | C(O)CH₃ | OH out of |
| | | | ring plane |

Other suitable anthracyclines are anthramycin, mitoxantrone, menogaril, nogalamycin, aclacinomycin A, olivomycin A, chromomycin A₃, and plicamycin having the structures:

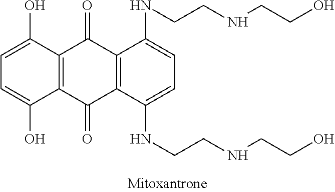

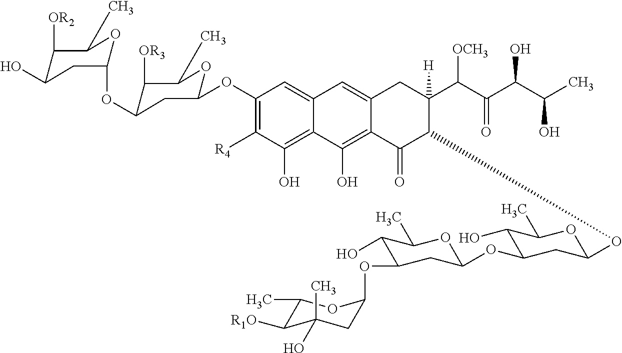

| | R₁ | R₂ | R₃ | R₄ |
|---|---|---|---|---|
| Olivomycin A | COCH(CH₃)₂ | CH₃ | COCH₃ | H |
| Chromomycin A₃ | COCH₃ | CH₃ | COCH₃ | CH₃ |
| Plicamycin | H | H | H | CH₃ |

| | R₁ | R₂ | R₃ |
|---|---|---|---|
| Menogaril | H | OCH₃ | H |
| Nogalamycin | O-sugar | H | COOCH₃ |

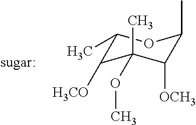

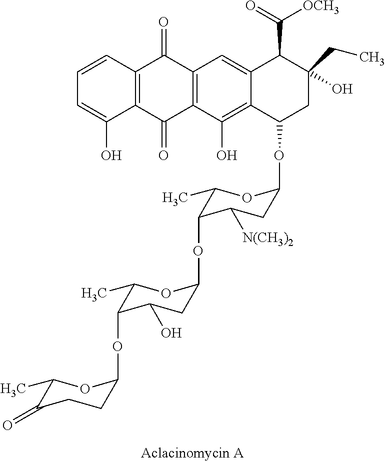

Other representative anthracyclines include, FCE 23762, a doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Nat'l Cancer Inst. 89(16):1217-1223, 1997), 4-demethoxy-7-O-[2,6-d]deoxy-4-O-(2,3,6-trideoxy-3-amino-α-L-lyxo-hexopyranosyl)-α-L-lyxo-hexopyranosyl]adriamicinone doxorubicin disaccharide analogue (Monteagudo et al., Carbohydr. Res. 300(1):11-16, 1997), 2-pyrrolinodoxorubicin (Nagy et al., Proc. Nat'l Acad. Sci. U.S.A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int. J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Willner et al., Bioconjugate Chem. 4(6):521-7, 1993), N-(5,5-diacetoxypent-1-yl) doxorubicin (Chemf & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ., 16(Biol. 1):21-7, 1988), 4′-deoxydoxorubicin (Schoelzel et al., Leuk. Res. 10(12):1455-9, 1986), 4-demethyoxy-4′-o-methyldoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20, 1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydroxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277).

ii. Fluoropyrimidine Analogues

In another aspect, the therapeutic agent is a fluoropyrimidine analog, such as 5-fluorouracil, or an analogue or derivative thereof, including carmofur, doxifluridine, emitefur, tegafur, and floxuridine. Exemplary compounds have the structures:

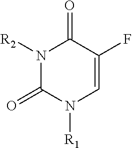

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

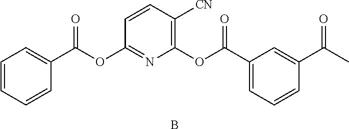

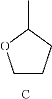

Other suitable fluoropyrimidine analogues include 5-FudR (5-fluoro-deoxyuridine), or an analogue or derivative thereof, including 5-iododeoxyuridine (5-IudR), 5-bromodeoxyuridine (5-BudR), fluorouridine triphosphate (5-FUTP), and fluorodeoxyuridine monophosphate (5-dFUMP). Exemplary compounds have the structures:

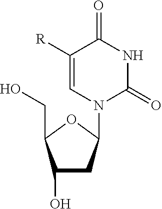

5-Fluoro-2′-deoxyuridine: R=F
5-Bromo-2′-deoxyuridine: R=Br
5-Iodo-2′-deoxyuridine: R=I

Other representative examples of fluoropyrimidine analogues include N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9, 1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3):144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-acetyl-3-O-toluoyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680).

These compounds are believed to function as therapeutic agents by serving as antimetabolites of pyrimidine.

iii. Folic Acid Antagonists

In another aspect, the therapeutic agent is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure:

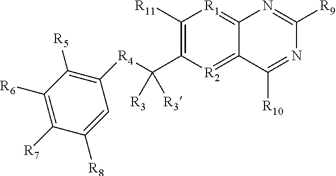

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure:

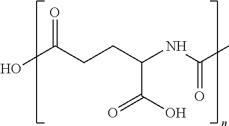

wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

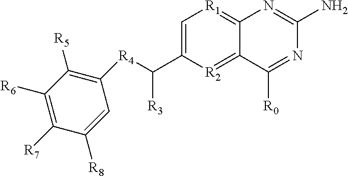

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatrexate | NH₂ | N | N | H | CH(CH₂CH₃) | H | H | A (n = 1) | H |
| Trimetrexate | NH₂ | CH | C(CH₃) | H | NH | H | OCH₃ | OCH₃ | OCH₃ |
| Pteropterin | OH | N | N | H | NH | H | H | A (n = 3) | H |
| Denopterin | OH | N | N | CH₃ | N(CH₃) | H | H | A (n = 1) | H |
| Peritrexim | NH₂ | N | C(CH₃) | H | single bond | OCH₃ | H | H | OCH₃ |

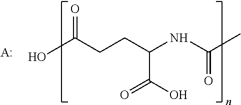

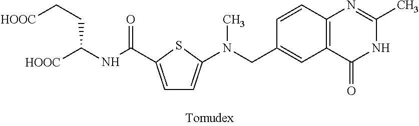

Other representative examples include 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7, 1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7, 1981); indoline ring and a modified ornithine or glutamic acid-bearing methotrexate derivatives (Matsuoka et al., Chem. Pharm. Bull. 45(7):1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaminopterin analogues (DeGraw et al., J. Med. Chem. 40(3):370-376, 1997), 5-deazaminopterin and 5,10-dideazaminopterin methotrexate analogues (Piper et al., J. Med. Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm. Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8, 1995), N-(α-aminoacyl) methotrexate derivatives (Cheung et al., Pteridines 3(1-2):101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(12):2400-3, 1991), β,γ-methano methotrexate analogues (Rosowsky et al., Pteridines 2(3):133-9, 1991), 10-deazaminopterin (10-EDAM) analogue (Braakhuis et al., Chem. Biol. Pteridines, Proc. Int. Symp. Pteridines Folic Acid Deriv, 1027-30, 1989), γ-tetrazole methotrexate analogue (Kalman et al., Chem. Biol. Pteridines, Proc. Int. Symp. Pteridines Folic Acid Deriv, 1154-7, 1989), N-(L-α-aminoacyl) methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed. Polym.):311-24, 1987), methotrexate-γ-dimyristoylphophatidylethanolamine (Kinsky et al., Biochim. Biophys. Acta 917(2):211-18, 1987), methotrexate polyglutamate analogues (Rosowsky et al., Chem. Biol. Pteridines, Pteridines Folid Acid Deriv., Proc. Int. Symp. Pteridines Folid Acid Deriv.: Chem., Biol. Clin. Aspects: 985-8, 1986), poly-γ-glutamyl methotrexate derivatives (Kisliuk et al., Chem. Biol. Pteridines, Pteridines Folid Acid Deriv., Proc. Int. Symp. Pteridines Folid Acid Deriv.: Chem., Biol. Clin. Aspects: 989-92, 1986), deoxyuridylate methotrexate derivatives (Webber et al., Chem. Biol. Pteridines, Pteridines Folid Acid Deriv., Proc. Int. Symp. Pteridines Folid Acid Deriv.: Chem., Biol. Clin. Aspects: 659-62, 1986), iodoacetyl lysine methotrexate analogue (Delcamp et al., Chem. Biol. Pteridines, Pteridines Folid Acid Deriv., Proc. Int. Symp. Pteridines Folid Acid Deriv.: Chem., Biol. Clin. Aspects: 807-9, 1986), 2,-omega-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122(Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (U.S. Pat. No. 4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.-Chim. Ther. 19(3):267-73, 1984), poly (L-lysine) methotrexate conjugates (Rosowsky et al., J. Med. Chem. 27(7):888-93, 1984), dilysine and trilysine methotrexate derivates (Forsch & Rosowsky, J. Org. Chem. 49(7):1305-9, 1984), 7-hydroxymethotrexate (Fabre et al., Cancer Res. 43(10):4648-52, 1983), poly-γ-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI (3):523-8, 1981), polyglutamate methotrexate derivatives (Galivan, Mol. Pharmacol. 17(1):105-10, 1980), halogentated methotrexate derivatives (Fox, JNCI (4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220);

These compounds are believed to act as antimetabolites of folic acid.

iv. Podophyllotoxins

In another aspect, the therapeutic agent is a Podophyllotoxin, or a derivative or an analogue thereof. Exemplary compounds of this type are etoposide or teniposide, which have the following structures:

| 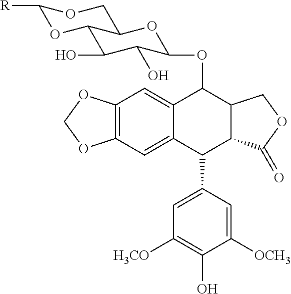 | |
|---|---|
| | R |
| Etoposide | CH₃ |
| Teniposide | 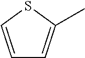 |

Other representative examples of podophyllotoxins include Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

These compounds are believed to act as topoisomerase II inhibitors and/or DNA cleaving agents.

v. Camptothecins

In another aspect, the therapeutic agent is camptothecin, or an analogue or derivative thereof. Camptothecins have the following general structure.

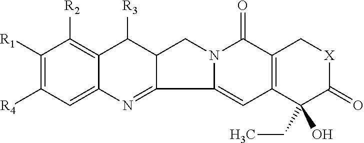

In this structure, X is typically O, but can be other groups, e.g., NH in the case of 21-lactam derivatives. R₁ is typically H or OH, but may be other groups, e.g., a terminally hydroxylated O₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylened ioxycamptothecin, SN-38, 9-nitrocamptothecin, 10-hydroxycamptothecin. Exemplary compounds have the structures:

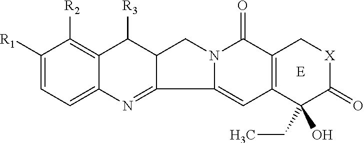

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

vi. Hydroxyureas

The therapeutic agent of the present invention may be a hydroxyurea. Hydroxyureas have the following general structure:

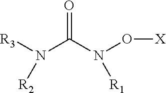

Suitable hydroxyureas are disclosed in, for example, U.S. Pat. No. 6,080,874, wherein R₁ is:

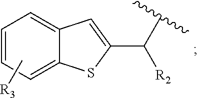

and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-[3-[5-(4-fluorophenylthio)-furyl]-2-cyclopenten-1-yl]N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with one or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form:

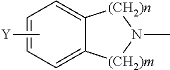

wherein m is 1 or 2, n is 0-2 and Y is an alkyl group.

In one aspect, the hydroxyurea has the structure:

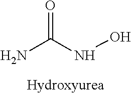

These compounds are thought to function by inhibiting DNA synthesis.

vii. Platinum Complexes

In another aspect, the therapeutic agent is a platinum compound. In general, suitable platinum complexes may be of Pt(II) or Pt(IV) and have this basic structure:

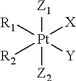

wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxyl, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

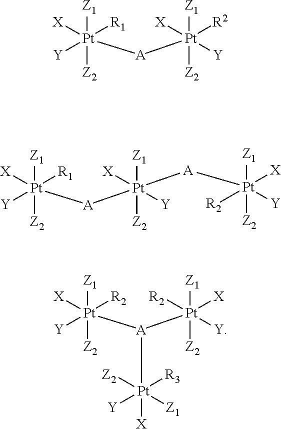

Exemplary platinum compounds are cisplatin, carboplatin, oxaliplatin, and miboplatin having the structures:

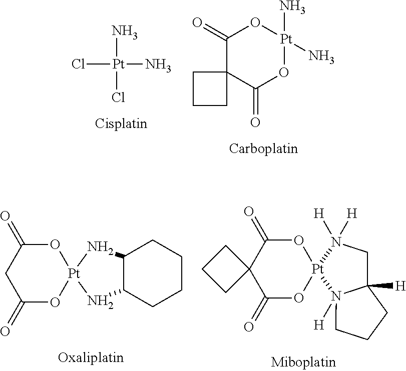

Other representative platinum compounds include (CPA)₂Pt[DOLYM] and (DACH)Pt[DOLYM] cisplatin (Choi et al., Arch. Pharmacal Res. 22(2):151-156, 1999), Cis-[PtCl₂(4,7-H-5-methyl-7-oxo]1,2,4[triazolo[1,5-a]pyrimidine)₂] (Navarro et al., J. Med. Chem. 41(3):332-338, 1998), [Pt(cis-1,4-DACH)(trans-Cl₂)(CBDCA)].½MeOH cisplatin (Shamsuddin et al., Inorg. Chem. 36(25):5969-5971, 1997), 4-pyridoxate diammine hydroxyl platinum (Tokunaga et al., Pharm. Sci. 3(7):353-356, 1997), Pt(II) . . . Pt(II) (Pt₂[NHCHN(C(CH₂)(CH₃))]₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer & Bednarski, J. Inorg. Biochem. 62(4):281-298, 1996), trans, cis-[Pt(Oac)₂I₂(en)] (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-[Pt(NH₃)(4-aminoTEMP-O){d(GpG)}] (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), CI-973 cisplatin analogue (Yang et al., Int. J. Oncol. 5(3):597-602, 1994), cis-diaminedichloroplatinum(II) and its analogues cis-1,1-cyclobutanedicarbosylato(2R)-2-methyl-1,4-butanediamineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bernays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol. 33(1):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexanemalonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2):157-65, 1987), JM8 and JM9 cisplatin analogues (Harstrick et al., Int. J. Androl. 10(1); 139-45, 1987), (NPr4)2((PtCL4).cis-(PtCl2-(NH2Me)2)) (Brammer et al., J. Chem. Soc., Chem. Commun. 6:443-5, 1987), aliphatic tricarboxylic acid platinum complexes (EPA 185225), and cis-dichloro(amino acid) (tert-butylamine)platinum(II) complexes (Pasini & Bersanetti, lnorg. Chim. Acta 107(4):259-67, 1985). These compounds are thought to function by binding to DNA, i.e., acting as alkylating agents of DNA.

viii. Combination Therapy

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate) and/or podophylotoxins (e.g., etoposide) can be utilized to enhance the antibacterial activity of the suture coating. Similarly anthracyclines (e.g., doxorubicin or mitoxantrone), fluoropyrimidines (e.g., 5-fluorouracil), folic acid antagonists (e.g., methotrexate) and/or podophylotoxins (e.g., etoposide) can be combined with traditional antibiotic and/or antifungal agents to enhance efficacy.

Dosages

As sutures are made in a variety of configurations and sizes, the exact dose of anti-infective agent administered will vary with suture size, length, diameter, surface area, design and portions of the suture coated. However, certain principles can be applied in the application of this art. Irrespective of the mechanism of action or pharmacological class of the drug, drug dose can be calculated as a function of dose per unit area (of the portion of the suture being coated), or total drug dose per suture. Total drug dose administered can be measured and appropriate surface concentrations of active drug can be determined. Regardless of the method of application of the drug to the suture, the preferred agents, used alone or in combination, should be administered under the following dosing guidelines:

(a) Anthracyclines Utilizing the anthracycline doxorubicin as an example, whether applied as a polymer coating, incorporated into the polymers which make up the suture (plain or self-retaining), or applied without a carrier polymer, the total dose of doxorubicin applied to the suture should not exceed 25 mg (range of 0.1 μg to 25 mg). In a particularly preferred embodiment, the total amount of drug applied should be in the range of 0.5 μg to 5 mg. The dose per unit area (i.e., the amount of drug as a function of the surface area of the portion of the suture to which drug is applied and/or incorporated) should fall within the range of 0.01 μg-100 μg per mm² of surface area. In a particularly preferred embodiment, doxorubicin should be applied to the suture surface at a dose of 0.1 μg/mm²-10 μg/mm². As different polymer and non-polymer coatings will release doxorubicin at differing rates, the above dosing parameters should be utilized in combination with the release rate of the drug from the suture surface such that a minimum concentration of 10⁻⁸-10⁻⁴ M of doxorubicin is maintained on the surface for the required duration of therapeutic effect. It is necessary to insure that surface drug concentrations exceed concentrations of doxorubicin known to be inhibitory to the growth of or lethal to multiple species of bacteria and/or fungi (i.e., are in excess of 10⁻⁴ M; although for some embodiments lower concentrations are sufficient). In a preferred embodiment, doxorubicin is released from the surface of the suture such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of doxorubicin (as described previously) with similar functional activity can be utilized for the purposes of this invention; the above dosing parameters are then adjusted according to the relative potency of the analogue or derivative as compared to the parent compound (e.g., a compound twice as potent as doxorubicin is administered at half the above parameters, a compound half as potent as doxorubicin is administered at twice the above parameters, etc.).

Utilizing mitoxantrone as another example of an anthracycline, whether applied as a polymer coating, incorporated into the polymers that make up the suture (plain or self-retaining), or applied without a carrier polymer, the total dose of mitoxantrone applied should not exceed 5 mg (range of 0.01 μg to 5 mg). In a particularly preferred embodiment, the total amount of drug applied should be in the range of 0.5 μg to 1 mg. The dose per unit area (i.e., the amount of drug as a function of the surface area of the portion of the suture to which drug is applied and/or incorporated) should fall within the range of 0.01 μg-20 μg per mm² of surface area. In a particularly preferred embodiment, mitoxantrone should be applied to the suture surface at a dose of 0.05 μg/mm²-3 μg/mm². As different polymer and non-polymer coatings will release mitoxantrone at differing rates, the above dosing parameters should be utilized in combination with the release rate of the drug from the suture surface such that a minimum concentration of 10⁻⁵-10⁻⁶ M of mitoxantrone is maintained for the required duration of therapeutic effect. It is necessary to insure that drug concentrations on the suture surface exceed concentrations of mitoxantrone known to be inhibitory to the growth of or lethal to multiple species of bacteria and/or fungi (i.e., are in excess of 10⁻⁵M; although for some embodiments lower drug levels will be sufficient). In a preferred embodiment, mitoxantrone is released from the surface of the suture such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of mitoxantrone (as described previously) with similar functional activity can be utilized for the purposes of this invention; the above dosing parameters are then adjusted according to the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Fluoropyrimidines Utilizing the fluoropyrimidine 5-fluorouracil as an example, whether applied as a polymer coating, incorporated into the polymers which make up the suture (plain or self-retaining), or applied without a carrier polymer, the total dose of 5-fluorouracil applied should not exceed 10 mg (range of 0.1 μg to 10 mg). In a particularly preferred embodiment, the total amount of drug applied should be in the range of 10 μg to 5 mg. The dose per unit area (i.e., the amount of drug as a function of the surface area of the portion of the suture to which drug is applied and/or incorporated) should fall within the range of 0.001 μg-0.1 mg per mm² of surface area. In a particularly preferred embodiment, 5-fluorouracil should be applied to the suture surface at a dose of 0.1 μg/mm²-50 μg/mm². As different polymer and non-polymer coatings will release 5-fluorouracil at differing rates, the above dosing parameters should be utilized in combination with the release rate of the drug from the suture surface such that a minimum concentration of 10⁻⁴-10⁻⁷ M of 5-fluorouracil is maintained for the required duration of therapeutic effect. It is necessary to insure that surface drug concentrations exceed concentrations of 5-fluorouracil known to be inhibitory to the growth of or lethal to numerous species of bacteria and/or fungi (i.e., are in excess of 10⁻⁴ M; although for some embodiments lower drug levels will be sufficient). In a preferred embodiment, 5-fluorouracil is released from the suture surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

(c) Podophylotoxins Utilizing the podophylotoxin etoposide as an example, whether applied as a polymer coating, incorporated into the polymers which make up the suture (plain or self-retaining), or applied without a carrier polymer, the total dose of etoposide applied should not exceed 15 mg (range of 0.1 μg to 15 mg). In a particularly preferred embodiment, the total amount of drug applied should be in the range of 1 μg to 2 mg. The dose per unit area (i.e., the amount of drug as a function of the surface area of the portion of the suture to which drug is applied and/or incorporated) should fall within the range of 0.01 μg-100 μg per mm² of surface area. In a particularly preferred embodiment, etoposide should be applied to the suture surface at a dose of 0.1 μg/mm²-10 μg/mm². As different polymer and non-polymer coatings will release etoposide at differing rates, the above dosing parameters should be utilized in combination with the release rate of the drug from the suture surface such that a concentration of 10⁻⁵-10⁻⁶M of etoposide is maintained for the required duration of therapeutic effect. It is necessary to insure that surface drug concentrations exceed concentrations of etoposide known to be inhibitory to the growth of or lethal to a variety of bacteria and/or fungi (i.e., are in excess of 10⁻⁵ M; although for some embodiments lower drug levels will be sufficient). In a preferred embodiment, etoposide is released from the surface of the suture such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of etoposide (as described previously) with similar functional activity can be utilized for the purposes of this invention; the above dosing parameters are then adjusted according to the relative potency of the analogue or derivative as compared to the parent compound (e.g., a compound twice as potent as etoposide is administered at half the above parameters, a compound half as potent as etoposide is administered at twice the above parameters, etc.).

2. Polymers

In certain embodiments, the compositions of the present invention may comprise polymers. Such polymers may function as polymeric carriers for, or control the release rates of, fibrosing agents or other therapeutic agents into the tissues surrounding the suture.

Representative examples of biodegradable polymers suitable for the use in conjunction with, and/or for the delivery of, fibrosing agents (and/or other therapeutic agents) include albumin, collagen, gelatin, hyaluronic acid, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, dextran sulfates, polysaccharides, sulfonated polysaccharides, fibrinogen, poly(ether ester) multiblock copolymers, based on poly(ethylene glycol) and poly(butylene terephthalate), tyrosine-derived polycarbonates (see, e.g., U.S. Pat. No. 6,120,491), poly(hydroxyl acids), poly(D,L-lactide), poly(D,L-lactide-co-glycolide), poly(glycolide), poly(hydroxybutyrate), poly(hydroxyvalerate), polydioxanone, poly(alkylcarbonate) and poly(orthoesters), aliphatic polyesters, poly(hydroxyvaleric acid), polydioxanone, poly(malic acid), poly(tartronic acid), poly(acrylamides), polyanhydrides, poly(ester-amides), poly(ester-imides), poly(ester-ureas), poly(ester-urethane-ureas), poly(anhydride-esters), poly(anhydride-imides), polyphosphazenes, poly(amino acids), poly(alkylene oxide)-poly(ester) block copolymers (e.g., X—Y, X—Y—X or Y—X—Y, R—(Y—X)ₙ, R—(X—Y)ₙ where X is a polyalkylene oxide and Y is a polyester (e.g., polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one.), R is a multifunctional initiator and copolymers as well as blends thereof. (see generally, Ilium, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986).

Representative examples of non-degradable polymers suitable for the use with, and delivery of, fibrosing agents (and/or other therapeutic agents) include poly(ethylene-co-vinyl acetate) (“EVA”) copolymers, silicone rubber, acrylic polymers (e.g., polyacrylic acid, polymethylacrylic acid, polymethylmethacrylate, poly(butyl methacrylate)), poly(alkylcyanoacrylate) (e.g., poly(ethylcyanoacrylate), poly(butylcyanoacrylate), poly(hexylcyanoacrylate), and poly(octylcyanoacrylate)), polyethylene, polypropylene, polyamides (nylon 6,6), polyurethanes (including hydrophilic polyurethanes), poly(ester-urethanes), poly(ether-urethanes), poly(ester-urea), poly(carbonate urethane)s, polyethers (poly(ethylene oxide), poly(propylene oxide), polyoxyalkylene ether block copolymers based on ethylene oxide and propylene oxide such as PLURONIC and PLURONIC R polymers, poly(tetramethylene glycol)), styrene-based polymers (polystyrene, poly(styrene sulfonic acid), poly(styrene)-block-poly(isobutylene)-block-poly(styrene), poly(styrene)-poly(isoprene) block copolymers], and vinyl polymers (polyvinylpyrrolidone, poly(vinyl alcohol), poly(vinyl acetate phthalate), as well as copolymers and blends thereof.

Polymers may also be developed which are either anionic (e.g., alginate, carrageenan, carboxymethyl cellulose, poly(acrylamido-2-methyl propane sulfonic acid) and copolymers thereof, poly(methacrylic acid) and copolymers thereof, and poly(acrylic acid) and copolymers thereof, as well as blends thereof) or cationic (e.g., chitosan, poly-L-lysine, polyethylenimine, and poly(allyl amine) and blends thereof (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995)).

In certain embodiments, polymers (including copolymers and blends of these polymers) include poly(ethylene-co-vinyl acetate), cellulose esters (nitrocellulose), poly(hydroxymethacrylate), poly(methylmethacrylate), poly(ethylene-co-acrylic acid), poly(vinylpyrrolidone) polyurethanes (e.g., CHRONOFLEX AL and CHRONOFLEX AR (both from CardioTech International, Inc., Woburn, Mass.) and BIONATE (Polymer Technology Group, Inc., Emeryville, Calif.)), poly(hydroxyl acids) (e.g., poly (D,L-lactic acid) oligomers and polymers, poly (L-lactic acid) oligomers and polymers, poly (glycolic acid), copolymers of lactic acid and glycolic acid, poly (caprolactone), and poly (valerolactone)), poly(anhydrides), poly(anhydride esters), poly(ester-amides), poly(ester-ureas), copolymers of poly (caprolactone) or poly (lactic acid) with a polyethylene glycol (e.g., MePEG), silicone rubbers, poly(styrene)block-poly(isobutylene)-block-poly(styrene), poly(acrylate) polymers, and blends, admixtures, or co-polymers of any of the above. Other examples (including copolymers and blends of these polymers) include poly(carbonate urethanes), poly(D-lactic acid) oligomers and polymers, copolymers of lactide and glycolide, copolymers of lactide or glycolide and ε-caprolactone, copolymers prepared from caprolactone and/or lactide and/or glycolide and/or polyethylene glycol. Other preferred polymers include collagen, poly(alkylene oxide)-based polymers, polysaccharides such as hyaluronic acid, chitosan and fucans, and copolymers of polysaccharides with degradable polymers, as well as crosslinked compositions of the above.

Further representative polymers for use in conjunction with a fibrosing agent (and/or another therapeutic agent) and/or that are capable of sustained localized delivery of fibrosing agents (and/or other therapeutic agents) include carboxylic polymers, polyacetates, polyacrylamides, polycarbonates, polyethers, substituted polyethylenes, polyvinylbutyrals, polysilanes, polyureas, polyoxides, polystyrenes, polysulfides, polysulfones, polysulfonides, polyvinylhalides, pyrrolidones, isoprene rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxies, melamines, other amino resins, phenolic polymers, and copolymers thereof, water-insoluble cellulose ester polymers (including cellulose acetate propionate, cellulose acetate, nitrocellulose, cellulose acetate butyrate, cellulose nitrate, cellulose acetate phthalate, and mixtures thereof), polyvinylpyrrolidone (pvp), polyethylene glycols, polyethylene oxides, polyvinyl alcohol, polyethers, poly(ethylene terephthalate), polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, nitro-cellulose, hydroxyethyl cellulose, cellulose nitrate, cellulose acetate, cellulose acetate butyrate, cellulose acetate propionate, polyacrylate, natural and synthetic elastomers, acetal, styrene polybutadiene, acrylic resin, polyvinylidene chloride, polycarbonate, homopolymers and copolymers of vinyl compounds, polyvinylchloride, and polyvinylchloride acetate.

Representative examples of patents relating to drug-delivery polymers and their preparation include PCT Publication Nos. WO 98/19713, WO 01/17575, WO 01/41821, WO 01/41822, and WO 01/15526 (as well as the corresponding U.S. applications), and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE37,950, 6,46,1631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, and 5,714,159, 5,612,052 and U.S. Published Patent Application Nos. 2003/0068377, 2002/0192286, 2002/0076441, and 2002/0090398. The polymers disclosed therein may also be useful in the present invention.

Polymeric carriers may be fashioned to release a fibrosing agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and acrylmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Additional exemplary polymers useful in the present invention include polymeric carriers that are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers, and the gelatin temperature [LCST (° C.)] include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof such as methylacrylic acid, acrylate and derivatives thereof such as butyl methacrylate, acrylamide, and N-n-butyl acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X—Y, Y—X—Y and X—Y—X wherein X in a polyalkylene oxide and Y is a biodegradable polyester (e.g., PLG-PEG-PLG) and PLURONICs such as F-127, 10-15° C.; L-122, 19° C.; L-92, 26° C.; L-81, 20° C.; and L-61, 24° C.

Representative examples of patents relating to thermally gelling polymers and the preparation include U.S. Pat. Nos. 6,451,346; 6,201,072; 6,117,949; 6,004,573; 5,702,717; and 5,484,610; and PCT Publication Nos. WO 99/07343; WO 99/18142; WO 03/17972; WO 01/82970; WO 00/18821; WO 97/15287; WO 01/41735; WO 00/00222 and WO 00/38651.

Within further aspects of the present invention, polymeric carriers are provided which are adapted to contain and release a hydrophobic fibrosing compound, and/or the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix that contains the hydrophobic fibrosing compound, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan and hyaluronic acid, proteins or polypeptides such as albumin, collagen and gelatin. Within alternative embodiments, hydrophobic compounds may be contained within a hydrophobic core, and this core contained within a hydrophilic shell.

Within further aspects, polymeric carriers can be materials that are formed in situ. In one embodiment, the precursors can be monomers or macromers that contain unsaturated groups that can be polymerized or crosslinked. The monomers or macromers can then, for example, be injected into the treatment area or onto the surface of the treatment area and polymerized or crosslinked in situ using a radiation source (e.g., visible light, UV light) or a free radical system (e.g., potassium persulfate and ascorbic acid or iron and hydrogen peroxide). The polymerization or crosslinking step can be performed immediately prior to, simultaneously to or post injection of the reagents into the treatment site. Representative examples of compositions that undergo free radical polymerization or crosslinking reactions are described in PCT Publication Nos. WO 01/44307, WO 01/68720, WO 02/072166, WO 03/043552, WO 93/17669, and WO 00/64977, U.S. Pat. Nos. 5,900,245; 6,051,248; 6,083,524; 6,177,095; 6,201,065; 6,217,894; 6,639,014; 6,352,710; 6,410,645; 6,531,147; 5,567,435; 5,986,043; and 6,602,975, and U.S. Patent Application Publication Nos. 2002/012796, 2002/0127266, 2002/0151650, 2003/0104032, 2002/0091229, and 2003/0059906.

In another embodiment, the reagents can undergo an electrophilic-nucleophilic reaction to produce a crosslinked matrix. Polymers terminated with nucleophilic groups such as amine, sulfhydryl, hydroxyl, —PH₂ or CO—NH—NH₂ can be used as the nucleophilic reagents and polymers terminated with electrophilic groups such as succinimidyl, carboxylic acid, aldehyde, epoxide, isocyanate, vinyl, vinyl sulfone, maleimid, —S—S—(C₅H₄N) or activated esters used in peptide synthesis can be used as the electrophilic reagents. For example, a 4-armed thiol derivatized poly(ethylene glycol) (e.g., pentaerythritol poly(ethylene glycol)ether tetra-succinimidyl glutarate) can be reacted with a 4 armed NHS-derivatized polyethylene glycol (e.g., pentaerythritol poly(ethylene glycol)ether tetra-sulfhydryl) under basic conditions (pH>about 8). Representative examples of compositions that undergo electrophilic-nucleophilic crosslinking reactions are described in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; U.S. Patent Application Publication No. 2003/0077272A1, and PCT Publication Nos. WO 2004/060405A2 and WO 2004/060346A2

In another embodiment, the electrophilic- or nucleophilic-terminated polymers can further comprise a polymer that can enhance the mechanical and/or adhesive properties of the in situ forming compositions. This polymer can be a degradable or non-degradable polymer. For example, the polymer may be collagen or a collagen derivative, for example methylated collagen. An example of an in situ forming composition uses pentaerythritol poly(ethylene glycol)ether tetra-sulfhydryl (4-armed thiol PEG), pentaerythritol poly(ethylene glycol)ether tetra-succinimidyl glutarate (4-armed NHS PEG) and methylated collagen as the reactive reagents. This composition, when mixed with the appropriate buffers will produce a crosslinked hydrogel.

In another embodiment, the polymer can be a polyester. Polyesters that can be used include the poly(hydroxyesters). In another embodiment, the polyester can comprise the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one. Representative examples of these types of compositions are described in U.S. Pat. Nos. 5,874,500; 5,936,035; 6,312,725; 6,495,127 and PCT Publication No. WO 2004/028547.

In another embodiment, the electrophilic-terminated polymer can be partially or completely replaced by a small molecule or oligomer that comprises an electrophilic group (e.g., disuccinimidyl glutarate).

In another embodiment, the nucleophilic-terminated polymer can be partially or completely replaced by a small molecule or oligomer that comprises a nucleophilic group (e.g., dicysteine, dilysine, trilysine, etc).

Other examples of in situ forming materials that can be used include those based on the crosslinking of proteins (described in U.S. Pat. Nos. RE38158; 4,839,345; 5,514,379, 5,583,114; 6,310,036; 6,458,147; 6,371,975; US Patent Application Publication Nos. 2004/0063613A1; 2002/0161399A1; 2001/0018598A1 and PCT Publication Nos. WO 03/090683; WO 01/45761; WO 99/66964 and WO 96/03159) and those based on isocyanate or isothiocyanate capped polymers (described in PCT Publication No. WO 04/021983). Adidtional examples of in situ formed polymers include those described in PCT Publication No. WO 05/051452.

Other examples of in situ forming materials can include reagents that comprise one or more cyanoacrylate groups. These reagents can be used to prepare a poly(alkylcyanoacrylate) or poly(carboxyalkylcyanoacrylate) (e.g., poly(ethylcyanoacrylate), poly(butylcyanoacrylate), poly(isobutylcyanoacrylate), poly(hexylcyanoacrylate), poly(methoxypropylcyanoacrylate) and poly(octylcyanoacrylate).

Examples of commercially available cyanoacrylates that can be used in conjunction with a fibrosing agent include DERMABOND, INDERMIL, GLUSTITCH, TISSUEMEND, VETBOND, TISSUMEND II, HISTOACRYL BLUE and ORABASE SOOTHE-N-SEAL LIQUID PROTECTANT or others as described above. Given the inherent adhesive properties of the cyanoacrylate polymers, they would be particularly useful alone, or in combination with a fibrosis-inducing agent, for use in association with self-retaining sutures.

In another embodiment, the cyanoacrylate compositions applied in association with the suture (particularly self-retaining sutures) can further comprise one or more additives to stabilize the reagents, or alter the rate of reaction of the cyanoacrylate, or alter the mechanical properties of the polymer or a combination thereof. For example, a trimethylene carbonate based polymer or an oxalate polymer of poly(ethylene glycol), or a ε-caprolactone based copolymer can be mixed with a 2-alkoxyalkylcyanoacrylate (e.g., 2-methoxypropylcyanoacrylate). Representative examples of these compositions are described in U.S. Pat. Nos. 5,350,798 and 6,299,631.

In another embodiment, the cyanoacrylate composition can be prepared by capping heterochain polymers with a cyanoacrylate group. The cyanoacrylate-capped heterochain polymer preferably has at least two cyanoacrylate ester groups per chain. The heterochain polymer can comprise an absorbable poly(ester), poly(ester-carbonate), poly(ether-carbonate) and poly(ether-ester). The poly(ether-ester)s described in U.S. Pat. Nos. 5,653,992 and 5,714,159 can also be used as the heterochain polymers. A triaxial poly(ε-caprolactone-co-trimethylene carbonate) is an example of a poly(ester-carbonate) that can be used. The heterochain polymer may be a polyether. Examples of polyethers that can be used include poly(ethylene glycol), poly(propylene glycol) and block copolymers of poly(ethylene glycol) and poly(propylene glycol) (e.g., PLURONICs polymers including, but not limited to, F127 or F68). Representative examples of these compositions are described in U.S. Pat. No. 6,699,940.

In addition to the compositions and methods described above, there are various other compositions and methods that are known in the art that may be used in making sutures that comprise fibrosing agents. Representative examples of these coating compositions and methods are described in U.S. Pat. Nos. 6,610,016, 6,358,557, 6,306,176, 6,110,483, 6,106,473, 5,997,517, 5,800,412, 5,525,348, 5,331,027, 5,001,009; 6,562,136; 6,406,754; 6,344,035; 6,254,921; 6,214,901; 6,077,698; 6,603,040; 6,278,018; 6,238,799; 6,096,726, 5,766,158, 5,599,576, 4,119,094; 4,100,309; 6,599,558; 6,369,168; 6,521,283; 6,497,916; 6,251,964; 6,225,431; 6,087,462; 6,083,257; 5,739,237; 5,739,236; 5,705,583; 5,648,442; 5645883; 5,556,710; 5,496,581; 4,689,386; 6,214,115; 6,090,901; 6,599,448; 6,054,504; 4,987,182; 4,847,324; and 4,642,267, U.S. Patent Application Publication Nos. 2003/0129130, 2001/0026834; 2003/0190420; 2001/0000785; 2003/0059631; 2003/0190405; 2002/0146581; 2003/020399; 2003/0129130, 2001/0026834; 2003/0190420; 2001/0000785; 2003/0059631; 2003/0190405; 2002/0146581; and 2003/020399, and PCT Publication Nos. WO 02/055121; WO 01/57048; WO 01/52915; and WO 01/01957.

It should be obvious to one of skill in the art that the polymers as described herein can also be blended or copolymerized in various compositions as required to deliver therapeutic doses of fibrosing agents to sites of suture placement.

3. Additional Components

Within certain embodiments of the invention, the fibrosing agents (or other therapeutic agents) can be delivered in association with suture placement using non-polymeric agents. These non-polymeric agents can include sucrose derivatives (e.g., sucrose acetate isobutyrate, sucrose oleate), sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydroxyapatite, zeolites; and combinations and mixtures thereof.

Representative examples of patents relating to non-polymeric delivery systems and the preparation include U.S. Pat. Nos. 5,736,152; 5,888,533; 6,120,789; 5,968,542; and 5,747,058.

Other carriers that may likewise be utilized to contain and deliver fibrosing agents described herein include: hydroxypropyl cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), nanoparticles (Violante and Lanzafame PAACR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), emulsion (Tarr et al., Pharm Res. 4: 62-165, 1987), and nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994).

In certain embodiments of the invention, fibrosing agents (or other therapeutic agents) can further comprise a secondary carrier. The secondary carrier can be in the form of microspheres or embolic particles (e.g., PLGA, PLLA, PDLLA, PCL, gelatin, polydioxanone, or poly(alkylcyanoacrylate)), nanospheres (e.g., PLGA, PLLA, PDLLA, PCL, gelatin, polydioxanone, or poly(alkylcyanoacrylate)), liposomes, emulsions, microemulsions, micelles (e.g., SDS, block copolymers of the form X—Y, X—Y—X or Y—X—Y where X is a poly(alkylene oxide) or alkyl ether thereof and Y is a polyester (e.g., PLGA, PLLA, PDLLA, PCL polydioxanone)), zeolites or cyclodextrins.

Within certain embodiments of the invention, the therapeutic compositions may also comprise additional ingredients such as surfactants (e.g., PLURONICS, such as F-127, L-122, L-101, L-92, L-81, and L-61), preservatives, and anti-oxidants.

Within certain embodiments of the invention, the fibrosing agent-containing compositions can also comprise radio-opaque, echogenic materials and magnetic resonance imaging (MRI) responsive materials (i.e., MRI contrast agents) to aid in visualization of sutures under ultrasound, fluoroscopy and/or MRI. For example, a suture may be made with or coated with a composition which is echogenic or radiopaque (e.g., made with echogenic or radiopaque with materials such as powdered tantalum, tungsten, barium carbonate, bismuth oxide, barium sulfate, metrazimide, iopamidol, iohexyl, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). Visualization of a suture by ultrasonic imaging may be achieved using an echogenic coating. Echogenic coatings are described in, e.g., U.S. Pat. Nos. 6,106,473 and 6,610,016. For visualization under MRI, contrast agents (e.g., gadolinium (III) chelates or iron oxide compounds) may be incorporated into or onto the suture, such as as a component in a coating. In some embodiments, a suture may include radio-opaque or MRI visible markers (e.g., bands) that may be used to orient and guide the suture during the implantation procedure.

In certain embodiments, sutures may be pre-attached to another device or element. For example, sutures may be pre-attached to a needle (e.g., CONTOUR THREAD®) or an anchor member for securing its placement in soft or hard tissues. In such embodiments, sutures themselves, the other devices or elements (e.g., needles and anchor members), or both the sutures and the other devices or elements may be coated with a radio-opaque, echogenic, or magnetic resonance imaging (MRI) responsive material.

Sutures may, alternatively, or in addition, be visualized under visible light, using fluorescence, or by other spectroscopic means. Visualization agents that can be included for this purpose include dyes, pigments, and other colored agents. In one aspect, the suture may further include a colorant to improve visualization of the suture in vivo and/or ex vivo. Frequently, sutures can be difficult to visualize upon insertion, especially at the margins of suture. A coloring agent can be incorporated into a suture to reduce or eliminate the incidence or severity of this problem. The coloring agent provides a unique color, increased contrast, or unique fluorescence characteristics to the suture. In one aspect, a suture is provided that includes a colorant such that it is readily visible (under visible light or using a fluorescence technique) and easily differentiated from its implant site. In another aspect, a colorant can be included in a liquid or semi-solid composition. For example, a single component of a two-component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan. Derivatives, analogues, and isomers of any of the above colored compound also may be used. The method for incorporating a colorant into a suture or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the composition and sutures of the present invention include one or more coloring agents, also referred to as dyestuffs, which will be present in an effective amount to impart observable coloration to the composition, e.g., the gel. Examples of coloring agents include dyes suitable for food such as those known as F. D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into a suture or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the compositions of the present invention include one or more preservatives or bacteriostatic agents present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of additional preservative include paraoxybenzoic acid esters, chlorobutanol, benzylalcohol, phenethyl alcohol, dehydroacetic acid, and sorbic acid. In one aspect, the compositions of the present invention include one or more bactericidal (also known as bacteriacidal) agents.

In one aspect, the compositions and sutures of the present invention include one or more antioxidants, present in an effective amount. Examples of the antioxidant include sulfites, alpha-tocopherol and ascorbic acid.

4. Physical Forms

The compositions of the present invention may be in various forms, such as microparticles or nanoparticles, microspheres, microcapsules, pastes, gels, sprays, and liquids. These can be applied to the surface of the suture or infiltrated into the tissues surrounding the suture. In certain embodiments, fibrosing agents may be linked by occlusion in the matrices of a polymer, bound by covalent linkages, bound by ionic interactions, or encapsulated in microcapsules.

Within certain aspects of the present invention, therapeutic compositions may be fashioned in any size ranging from 20 nm to 1500 μm, depending upon the particular use. These compositions can be in the form of microspheres (porous or non-porous), microparticles and/or nanoparticles. These compositions can be formed by spray-drying methods, milling methods, coacervation methods, W/O (water-oil) emulsion methods, W/O/W emulsion methods, and solvent evaporation methods. In another embodiment, these compositions can include microemulsions, emulsions, liposomes and micelles. Alternatively, such compositions may also be readily applied as a “spray”, which solidifies into a film or coating for use as a suture surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions of the present invention may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing fibrosing agents are particularly useful for application to the surface of tissues that will be in contact with the suture.

C. Sutures that Comprise Fibrosing Agents

Various types of sutures (plain or self-retaining) may be used in combination with fibrosing agents according to the present invention. In certain embodiments, sutures are absorbable (e.g., those that are degraded by the body's enzymatic pathways and generally lose tensile strength by 60 days after implantation). In certain embodiments, the absorbable sutures are made of polymers or copolymers of glycolic and lactic acid. Exemplary absorbable sutures include catgut (both plain and chromic) (e.g., those with a trade name PROGUT from Dolphin Sutures, India), and those derived from polyglycolic acid with a trade name PETCRYL (Dolphin Sututes, India) and with a trade name DEXON™ (Sherwood Services AG, Schaffhausen, Switzerland), from poliglecaprone 25 with a trade name MONOCRYL® (copolymer of about 75% glycolide and about 25% caprolactone, Johnson & Johnson Co., New Brunswick, N.J.), from polyglactin 910 (such as VICRYL®, coated VICRYL®, coated VICRYL® Plus Antibacterial sutures that contain antibacterial triclosan, and Coated VICRYL RAPIDE® sutures, Johnson & Johnson Co., New Brunswick, N.J.), MULTIPASS® Needle Coating (Johnson & Johnson Co., New Brunswick, N.J.), copolymer of about 67% glycolide and about 33% trimethylene carbonate sold as MAXON™, Wyeth, Madison, N.J., and from polydioxanone with a trade name PDS II® (Johnson & Johnson Co., New Brunswick, N.J.).

In addition to the sutures described above, degradable sutures can be made from polymers such as polyglycolic acid, copolymers of glycolide and lactide, copolymers of trimethylene carbonate and glycolide with diethylene glycol (e.g., MAXON™, Tyco Healthcare Group), terpolymer composed of glycolide, trimethylene carbonate, and dioxanone (e.g., BIOSYN™ [glycolide (60%), trimethylene carbonate (26%), and dioxanone (14%)], Tyco Healthcare Group), copolymers of glycolide, caprolactone, trimethylene carbonate, and lactide (e.g., CAPROSYN™, Tyco Healthcare Group). Other sutures that can be used in this invention include sutures composed of a polymer that comprises the residues of one or more of the monomers selected from lactide, lactic acid, glycolide, glycolic acid, e-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, 1,4-dioxane-2-one, and 1,5-dioxepan-2-one. These sutures can be in either a braided multifilament form or a monofilament form. The polymers used in the present invention can be linear polymers, branched polymers or multi-axial polymers. Examples of multi-axial polymers used in sutures are described in U.S. Patent Application Publication Nos. 20020161168, 20040024169, and 20040116620.

Absorbable sutures may be used below the surface of the skin to provide support to the skin closure. They may also be used in areas where suture removal might jeopardize the repair such as with small children who might not easily cooperate with suture removal.

In certain embodiments, sutures that may be used in combination with fibrosing agents are non-absorbable, which may be of monofilament and braided types. Non-absorbable sutures are permanent and include sutures made of polyamide (also known as nylon, such as nylon 6 and nylon 6.6), polyester (e.g., polyethylene terephthlate), polytetrafluoroethylene (e.g., expanded polytetrafluoroethylene), polyether-ester such as polybutester (block copolymer of butylene terephthalate and polytetra methylene ether glycol), polyurethane, metal alloys, metal (e.g., stainless steel wire), polypropylene, polyethelene, silk, and cotton. Exemplary non-absorbable sutures include coated polyester sutres with a trade name Procare (Dolphin Sutures, India), GORTEX™ (made of expanded polytetrafluoroethylene, sold by Gore), NOVAFIL™ (made of polybutester, Wyeth, Madison, N.J.), monofilament polyamide sutures with a trade name Linex (Dolphin Sutures, India), SUTURA® (black braided silk sutures, Sutura Inc., Fountain Valley, Calif.), monofilament polypropylene sutures with a trade name Duracare (Dolphin Sutures, India), MONOSOF® (monofilament nylon suture, United States Surgical Co., Norwalk, Conn.), DERMALON™ (monofilament nylon suture, Sherwood Services AG, Switzerland), SURGILON™ (braided nylon suture coated with silicone, Sherwood Services AG, Switzerland), Ethilon nylon suture (Ethicon, Inc., Somerville, N.J.), ETHIBOND EXCEL® (braided polyester suture from Johnson & Johnson Co., New Brunswick, N.J.), Pronova poly(hexafluoropropylene-VDF) suture (Ethicon, Inc. Somerville, N.J.), TEVDEK™ (braided polyester suture from J.A. Deknatel and Son, Inc. New York, N.Y.), PROLENE™ (polypropylene suture from Ethicon, Inc., Somerville, N.J.), FLUOROFIL™ (polypropylene suture from Pitman-Moore, Inc. Lake Forest, Ill.), and MERSILENE™ (polyester fiber suture from Ethicon, Inc., Somerville, N.J.).

In embodiments wherein a suture is predominately silk (i.e., silk constitutes over 50% of the suture by weight), another fibrosing agent (i.e., a fibrosing agent other than silk) is also included in the suture according to the present invention. In other words, the present invention does not include silk sutures (i.e., sutures composed of silk) that do not contain one or more other fibrosing agents.

Additional exemplary sutures that may be used in combination with fibrosing agents according to the present invention are various sutures available from Surgical Specialities Co. (Reading, Pa.), including monoderm undyed or dyed monofilament sutures, clear or dyed PCL monofilament sutures, dyed polypropylene monofilament sutures, undyed braided POLYSYN FA sutures, dyed or undyed braided PGA sutures, dyed or undyed braided polysyn suture, dyed monofilament polysyn sutures, dyed braided poloyester sutures, braided silk sutures, dyed braided polyviolene sutures, plain or chromic gut sutures, dyed or undyed monofilament nylon sutures, and dyed pliable nylon sutures.

Additional exemplary sutures that may be used in combination with fibrosing agents according to the present invention are various sutures available from Tyco International Ltd., Bermuda or its companies. Such sutures include SURGITIE™ (single use ligating loops with delivery system) and SURGIWIP™ (single use sutrue ligatures with delivery system), absorbable sutures such as POLYSORB™ (suturtes composed of LACTOMER™ glycolide/lactide copolymer, a synthetic polyester composed of glycolide and lactide (derived from glycolic and lactic acids), DEXON™ II (synthetic suture composed of homopolymer of glycolic acid and coated with POLYCAPROLATE™, a copolymer of glycolide and epsilon-caprolactone), DEXON™ S (synthetic sutures composed of the homopolymer of glycolic acid), MAXON™ CV (polyglyconate synthetic sutures prepared from a copolymer of glycolic acid and trimethylene carbonate), plain, mild chromic, and chromic gut sutures composed of purified connective tissue (mostly collagen) derived from the serosal layer of beef intestines, and non-absorbable sutures such as DERMALON® (nylon), MONOSOF® (nylon), SURGILON® (nylon), SURGIDAC™ (polyethylene terephthalate), TI-CRON™ (sutures prepared from fibers of high molecular weight, long chain and linear polyesters having recurrent aromatic rings as an intergral component), SURGIPRO™ (sutures composed of an isotactic crystalline steroisomer of polypropylene (a synthetic linear polyolefin) and polyethylene), SURGIPRO™ II (sutures composed of an isotactic crystalline steroisomer of polypropylene (a synthetic linear polyolefin) and polyethylene), NOVAFIL™ (sutures composed of polybutester, a copolymer of butylenes terephthalate and polytetramethylene ether glycol), VASCUFIL™ (sutures composed of a copolymer of butylenes terephthalate and polytetramethylene ether glycol and coated with POLYTRIBOLATE™, an absorbable polymer of ε-caprolactone/glycolide/poloxamer 188), FLEXON™ (twisted multistrand steel sutures coated with orange or white PTFE poly(tetrafloroproethylene) or clear FEP poly(tetrafluoroethylene-co-hexafluoropropylene), SOFSILK™ (sutures composed of natural proteinaceous silk fibers that are treated to remove the naturally-occurring sericin gum), and stainless steel sutures.

In certain embodiments, sutures that may be used in combination with fibrosing agents are used for joining tissue in surgical procedures, including, without limitation, joining and holding closed a wound (such as a surgical incision) in bodily tissue, fastening junctions of wounds, tying off wounds, and joining a foreign element to tissue.

In certain embodiments, sutures that may be used in combination with fibrosing agents are used in various dental procedures, i.e., oral and maxillofacial surgical procedures and thus may be referred to as “dental sutures.” The above-mentioned procedures include, but are not limited to, oral surgery (e.g., removal of impacted or broken teeth), surgery to provide bone augmentation, surgery to repair dentofacial deformities, repair following trauma (e.g., facial bone fractures and injuries), surgical treatment of odontogenic and non-odontogenic tumors, reconstructive surgeries, repair of cleft lip or cleft palate, congenital craniofacial deformities, and esthetic facial surgery. Many of the various sutures described above are used in such procedures and are available from many of the same commercial sources. As above, dental sutures may be degradable or non-degradable. Sutures used in oral and maxillofacial surgical procedures may typically range in size from USP 2-0 to USP 6-0. Dental sutures may have a surgical needle attached.

In certain embodiments, sutures that may be used in combination with fibrosing agents are microsutures. Microsutures are used in microsurgical procedures that are performed under a surgical microscope. Such surgical procedures include, but are not limited to, reattachment and repair of peripheral nerves, spinal microsurgery, microsurgery of the hand, various plastic microsurgical procedures (e.g., facial reconstruction), microsurgery of the male or female reproductive systems, and various types of reconstructive microsurgery. Microsurgical reconstruction is used for complex reconstructive surgery problems when other options such as primary closure, healing by secondary intention, skin grafting, local flap transfer, and distant flap transfer are not adequate. Microsutures are available from many of the commercial sources identified above and are made from the same materials described above. As above, microsutures may be degradable or non-degradable. Microsutures have a very small caliber, often as small as USP 9-0 or USP 10-0, and may have an attached needle of corresponding size.

Additional exemplary sutures that may be used in combination with fibrosing agents are described in U.S. Pat. Nos. 5,766,188, 4,441,496, 6,692,516, 4,550,730, 4,052,988, and U.S. Patent Application Publication Nos. 2005267532, 2005240224, 2004111116, 2004088003, 2002095180.

In certain embodiments, the suture can further comprise a coating. The coating can comprise a degradable or a non-degradable polymer. Coatings can include but are not limited to polybutylene adipate (SURGIDAC™) to silicone (TI-CRON™), poly(glycolide-co-lactide) (e.g., polyglactin 370), copolymers of gylcolide, ε-caprolactone, and poloxamer 188, copolymers of glycolide and, ε-caprolactone (e.g., polycaprolate), poloxamer 188, calcium stearate, and calcium stearoyl lactylate, as well as blends and mixtures thereof. Coatings can also include polymers that can comprise the residues of one of more of the monomers selected from lactide, lactic acid, glycolide, glycolic acid, ε-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, 1,4-dioxane-2-one, and 1,5-dioxepan-2-one. Exemplary coatings that can be used are described in U.S. Pat. Nos. 4,047,533, 4,201,216, 4,470,416, 4,788,979, 4,857,602, 4,994,074, 5,037,950, 5,100,433, 5,102,420, 5,123,912, 5,522,842, 5,543,218, 6,703,035, and 6,703,035. In certain embodiments, the fibrosis-inducing agent can be incorporated into or onto the suture coating polymers described above.

In certain particularly preferred embodiments, sutures that may be used in combination with fibrosing agents are self-retaining sutures. Such sutures have the following advantages over sutures without retainers: providing self-anchoring capability, allowing reduced surgical training in knot tying, faster placement, permitting approximation of difficult to sew tissues (e.g., muscle, liver, speen, and renal tissue) and enhancing healing by, for example, increasing blood flow. Bi-directional self-retaining sutures may further permit tissues to be approximated and held taut during suturing, which is especially advantageous in closing long incisions or large wounds.

Self-retaining sutures that may be used in combination with fibrosing agents include but are not limited to: one-way sutures disclosed in U.S. Pat. Nos. 3,123,077, 5,053,047, 5,931,855, PCT Application Publication No. WO 98/52473, barbed suture described in McKenzie et al., J Bone Joint Surg [Br] 49(3): 440-7, 1967, and bi-directional suture described in U.S. Pat. Nos. 5,342,376, 6,241,747, US 2003/0074023, and Dattilo et al., 2003 Society For Biomaterials 29^{th} Annual Meeting Transactions. Page 101. Additional description of self-retaining sutures useful in the present invention may be found in U.S. Pat. Nos. 6,599,310, 6,773,450, 6,848,152, published U.S. Application Nos. US 2004/0060410, US 2004/0060409, US 2004/0088003, and US 2004/0226427, and PCT Application Publication Nos. WO 03/001979, WO 2004/014236, WO 03/017850, WO 2004/030520, WO 2004/030704, WO 2004/030705, and WO 2007/005296.

In one embodiment, a particularly preferred, commercially available suture that may be used in combination with a fibrosing agent is CONTOUR THREADS™ (Quill Medical, Research Triangle Park, N.C.). CONTOUR THREADS™ are non-absorbable self-retaining suture product cleared by the FDA for the elevation and fixation of midface, brow and neck areas. They are made from clear polypropylene.

In another embodiment, the suture that may be used in combination with a fibrosing agent is a suture with a similar structure to the CONTOUR THREADS™ but is composed of a degradable polymer. These degradable polymers can comprise the residues of one or more of the monomers selected from lactide, lactic acid, glycolide, glycolic acid, e-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, 1,4-dioxane-2-one, and 1,5-dioxepan-2-one. Examples include polydioxanone, poly(lactide-co-trimethylene carbonate) polymers, poly(lactide-co-glycolide) polymers, and poly(lactide-co-glycolide-co-trimethylene carbonate) polymers.

In other embodiments, the suture that may be used in combination with a fibrosing agent is Aptos Thread developed by Dr. Sulamandize of Moscow (available from I-LIFT TENSOR THREADS, Argentine). Description of such suture may be found in European Published Patent Application No. 1,075,843 A1 and WO 00/51658. The suture has conical retainers arranged sequentially along the length of a thread and orented in a direction opposite to that of the thread tension, with the distance between retainers being no less than 1.5 times the thread diameter.

As indicated above, certain types of self-retaining sutures that may be used in combination with a fibrosing agent according to the present invention are commercially available. In certain other embodiments, self-retaining sutures may be made using any suitable method, including injection molding, stamping, cutting, laser, extrusion, separate manufacture and subsequent attachment of retainers, and the like. With respect to cutting, polymeric thread or filaments may be purchased, and the retainers are subsequently cut onto the filament body. In certain embodiments, self-retaining sutures may be produced according to U.S. Pat. No. 6,848,152 and U.S. Patent Application Publication Nos. US 2004/0226427 and US 2004/0060409.

In certain embodiments, self-retaining sutures that may be used in combination with fibrosing agents are used in tissue repositioning surgical procedures. Such surgical procedures include, without limitation, facelifts, neck lifts, brow lifts, thigh lifts, and breast lifts. Self-retaining sutures used in tissue repositioning procedures may vary depending on the tissue being repositioned; for example, sutures with larger and further spaced-apart retainers may be suitably employed with relatively soft tissues such as fatty tissues.

In certain embodiments, sutures that may be used in combination with a fibrosing agent according to the present invention are already attached to surgical needles. Attachment of sutures and surgical needles is described in U.S. Pat. Nos. 3,981,307, 5,084,063, 5,102,418, 5,123,911, 5,500,991, 5,722,991, 6,012,216, and 6,163,948, and U.S. Patent Application Publication No. US 2004/0088003. A method for the manufacture of surgical needles is described in U.S. Pat. No. 5,533,982, and a method for the manufacture of polymer-coated surgical needles is described in U.S. Pat. No. 5,258,013.

In certain embodiments, the sutures that may be used in combination with a fibrosing agent according to the present invention are pointing at both ends (including suture connectors as described in U.S. Pat. No. 6,241,747). In certain other embodiments, the sutures may have one pointing end and an achor on the other end. The anchor may be used to secure the implantation of the suture in soft tissue (e.g., those described in U.S. Patent Application Publication No. US2005/0267531) or the attachment of sutures to the bone (e.g., those described in U.S. Pat. No. 6,773,450 and PCT Application Publication No. WO 2004/014236).

In certain other embodiments, the suture may be a relatively short suture with sharp pointing ends. Such a suture may function similar to a staple when used in connecting tissues and thus permits a surgeon to rapidly and securely attach the edges of a wound in a bodily tissue or reconfigure the tissue without the necessity for threading and tying numerous individual stitches or for the use of a complicated tool to insert the suture. This type of sutures may thus be referred to as “suture connector.” In certain embodiments, the suture connector may be a bi-directional self-retaining suture. In certain other embodiments, the suture connector may be found by linking two relatively short uni-directional self-retaining sutures together to form a bi-directional self-retaining suture (see, U.S. Pat. No. 6,241,747).

D. Methods for Making Sutures that Comprise Fibrosing Agents

Various methods may be used to make sutures that comprise fibrosing agents. For example, such methods may comprise the step of coating (e.g., spraying or dipping) all or part of the sutures. Additionally, sutures themselves may be comprised at least in part of materials (e.g., silk) that induce or stimulate fibrosis in or around the site where the sutures are implanted or inserted.

In certain embodiments, only selected portions (such as middle sections or the self-retaining sections) of sutures may be coated or otherwise comprise fibrosing agents or fibrosing agent-containing compositions. In certain further embodiments, portions of the sutures may be selectively comprised (at least in part) of fibrosing or fibrosing agent-containing compositions. In certain other embodiments, the suture surface may comprise one or more wells of fibrosing or fibrosing agent-containing compositions. In other embodiments, all sections of sutures may be coated or otherwise comprise fibrosing agents or fibrosing agent-containing compositions.

1. Exemplary Methods for Combining Fibrosing Agents with Sutures

Various exemplary methods for combining fibrosing agents with sutures to produce sutures that comprise fibrosing agents are described in more detail below.

a. Coating of Sutures with Fibrosing Agents

Fibrosing agents or compositions comprising fibrosing agents may be coated onto or into a suture by various methods known in the art such as by dipping, spraying, electrospinning, painting or vacuum deposition. In certain embodiments including self-retaining sutures, the retainers may be coated during or after their formation on the sutures during the manufacturing process (thereby resulting in sutures having selectively uncoated portions). In certain other embodiments, including self-retaining sutures, the entire sutures may be coated during manufacture.

i. Dip Coating

Dip coating is one coating process that can be used to coat a suture. In one embodiment, the fibrosing agent is dissolved in a solvent for the fibrosing agent and is then coated onto the suture.

Fibrosing Agent with an Inert Solvent

In one embodiment, the solvent is an inert solvent for the suture such that the solvent does not dissolve the suture to any significant extent and is not absorbed by the suture to any significant extent. The suture can be immersed, either partially or completely, in the fibrosing agent/solvent solution for a specific period of time. The rate of immersion into the fibrosing agent/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. This process will result in the fibrosing agent being coated on the surface of the suture.

Fibrosing Agent with a Swelling Solvent

In one embodiment, the solvent is one that will not dissolve the suture but will be absorbed by the suture. These solvents can thus swell the suture to some extent. The suture can be immersed, either partially or completely, in the fibrosing agent/solvent solution for a specific period of time (seconds to days). The rate of immersion into the fibrosing agent/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. This process will result in the fibrosing agent being adsorbed into the suture. The fibrosing agent may also be present on the surface of the suture. The amount of surface associated fibrosing agent may be reduced by dipping the coated suture into a solvent for the fibrosing agent or by spraying the coated suture with a solvent for the fibrosing agent.

Fibrosing Agent with a Solvent

In one embodiment, the solvent is one that will be absorbed by a suture and that will dissolve the suture. The suture can be immersed, either partially or completely, in the fibrosing agent/solvent solution for a specific period of time (seconds to hours). The rate of immersion into the fibrosing agent/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. This process will result in the fibrosing agent being adsorbed into the suture as well as being surface associated. In the preferred embodiment, the exposure time of the suture to the solvent would be such that the suture does not undergo significant permanent dimensional changes. The fibrosing agent may also be present on the surface of the suture. The amount of surface associated fibrosing agent may be reduced by dipping the coated suture into a solvent for the fibrosing agent or by spraying the coated suture with a solvent for the fibrosing agent.

In the above description, the suture can be a suture that has not been modified as well as a suture that has been further modified by coating with a polymer (e.g., parylene), surface treated, surface etching, mechanical smoothing or roughening, or grafting prior to the coating process.

In one embodiment, the fibrosing agent and a polymer are dissolved in a solvent, for both the polymer and the fibrosing agent, and are then coated onto the suture.

Fibrosing Agent/Polymer with an Inert Solvent

In one embodiment, the solvent is an inert solvent for the suture such that the solvent does not dissolve the suture to any great extent and is not absorbed by the suture to any great extent. The suture can be immersed, either partially or completely, in the fibrosing agent/polymer/solvent solution for a specific period of time. The rate of immersion into the fibrosing agent/polymer/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. This process will result in the fibrosing agent/polymer being coated on the surface of the suture.

Fibrosing Agent/Polymer with a Swelling Solvent

In one embodiment, the solvent is one that will not dissolve a suture but will be absorbed by the suture. These solvents can thus swell the suture to some extent. The suture can be immersed, either partially or completely, in the fibrosing agent/polymer/solvent solution for a specific period of time (seconds to days). The rate of immersion into the fibrosing agent/polymer/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. This process will result in the fibrosing agent/polymer being coated onto the surface of the suture as well as the potential for the fibrosing agent being adsorbed into the suture. The fibrosing agent may also be present on the surface of the suture. The amount of surface associated fibrosing agent may be reduced by dipping the coated suture into a solvent for the fibrosing agent or by spraying the coated suture with a solvent for the fibrosing agent.

Fibrosing Agent/Polymer with a Solvent

In one embodiment, the solvent is one that will be absorbed by a suture and that will dissolve the suture. The suture can be immersed, either partially or completely, in the fibrosing agent/solvent solution for a specific period of time (seconds to hours). The rate of immersion into the fibrosing agent/solvent solution can be altered (e.g., 0.001 cm per sec to 50 cm per sec). The suture can then be removed from the solution. The rate at which the suture can be withdrawn from the solution can be altered (e.g., 0.001 cm per sec to 50 cm per sec). The coated suture can be air-dried. The dipping process can be repeated one or more times depending on the specific application. The suture can be dried under vacuum to reduce residual solvent levels. In the preferred embodiment, the exposure time of the suture to the solvent would be such that there is not significant permanent dimensional change to the suture (other than those associated with the coating itself). The fibrosing agent may also be present on the surface of the suture. The amount of surface associated fibrosing agent may be reduced by dipping the coated suture into a solvent for the fibrosing agent or by spraying the coated suture with a solvent for the fibrosing agent.

In the above description the suture can be a suture that has not been modified as well as a suture that has been further modified by coating with a polymer (e.g., parylene), surface treated, surface etching, mechanical smoothing or roughening, or grafting prior to the coating process.

In any one of the above dip coating methods, the surface of the suture can be treated with a plasma polymerization method prior to coating of the fibrosing agent or fibrosing agent containing composition, such that a thin polymeric layer is deposited onto the suture surface. Examples of such methods include parylene coating of sutures and the use of various monomers such hydrocyclosiloxane monomers. Parylene coating may be especially advantageous if the suture, or portions of the suture, is composed of materials (e.g., stainless steel, nitinol) that do not allow incorporation of the therapeutic agent(s) into the surface layer using one of the above methods. A parylene primer layer may be deposited onto the electrical suture using a parylene coater (e.g., PDS 2010 LABCOTER2 from Cookson Electronics, Inc., Foxborough, Mass.) and a suitable reagent (e.g., di-p-xylylene or dichloro-di-p-xylylene) as the coating feed material. Parylene compounds are commercially available, for example, from Specialty Coating Systems, Indianapolis, Ind.), including PARYLENE N (di-p-xylylene), PARYLENE C (a monchlorinated derivative of PARYLENE N, and PARYLENE D, a dichlorinated derivative of PARYLENE N).

In another embodiment, a suspension of the fibrosing agent in a polymer solution can be prepared. The suspension can be prepared by choosing a solvent that can dissolve the polymer but not the fibrosing agent or a solvent that can dissolve the polymer and in which the fibrosing agent is above its solubility limit. In similar processes described above, a suture can be dipped into the suspension of the fibrosing agent and polymer solution such that the suture is coated with the suspension.

Bath Coating Process

In one embodiment, the active agent can be dissolved or suspended in a solution of a polymer that will be used to coat the suture (suture coatings as described above). The suture can then be passed through the bath containing the coating polymer solution/active agent in either a continuous manner or in a batch process.

ii. Spray Coating

Spray coating is another coating process that can be used. In the spray coating process, a solution or suspension of the fibrosing agent, with or without a polymeric or non-polymeric carrier, is nebulized and directed to the suture to be coated by a stream of gas. One can spray coat sutures using equipment such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part # 14545 and 14654, Alltech Associates, Inc. Deerfield, Ill.), and ultrasonic atomizers (for example those available from Sono-Tek, Milton, N.Y.). One can also use powder sprayers and electrostatic sprayers.

In one embodiment, the fibrosing agent is dissolved in a solvent for the fibrosis agent and is then sprayed onto the suture.

Fibrosing Agent with an Inert Solvent

In one embodiment, the solvent is an inert solvent for a suture such that the solvent does not dissolve the suture to any great extent and is not absorbed by the suture to any great extent. The suture can be held in place or the suture can be mounted onto a mandrel or rod that has the ability to move in an X, Y or Z plane or a combination of these planes. Using one of the above described spray coaters, the suture can be spray coated such that the suture is either partially (e.g., coating of the self-retaining region only) or completely coated with the fibrosing agent/solvent solution. The rate of spraying of the fibrosing agent/solvent solution can be altered (e.g., 0.001 ml per sec to 10 ml per sec) to ensure that a good coating of the fibrosing agent is obtained. The coated suture can be air-dried. The spray coating process can be repeated one or more times depending on the specific application. The suture can be dried under vacuum to reduce residual solvent levels. This process will result in the fibrosing agent being coated on the surface of the suture.

Fibrosing Agent with a Swelling Solvent

In one embodiment, the solvent is one that will not dissolve a suture but will be absorbed by the suture. These solvents can thus swell the suture to some extent. The suture can be spray coated, either partially (e.g., coating of the self-retaining region only) or completely, in the fibrosing agent/solvent solution. The rate of spraying of the fibrosing agent/solvent solution can be altered (e.g., 0.001 ml per sec to 10 ml per sec) to ensure that a good coating of the fibrosing agent is obtained. The coated suture can be air-dried. The spray coating process can be repeated one or more times depending on the specific application. The suture can be dried under vacuum to reduce residual solvent levels. This process will result in the fibrosing agent being adsorbed into the suture. The fibrosing agent may also be present on the surface of the suture. The amount of surface associated fibrosing agent may be reduced by dipping the coated suture into a solvent for the fibrosing agent or by spraying the coated suture with a solvent for the fibrosing agent.

Fibrosing Agent with a Solvent

In one embodiment, the solvent is one that will be absorbed by a suture and that will dissolve the suture. The suture can be spray coated, either partially (for example, coating of the self-retaining region only) or completely, in the fibrosing agent/solvent solution. The rate of spraying of the fibrosing agent/solvent solution can be altered (e.g., 0.001 ml per sec to 10 ml per sec) to ensure that a good coating of the fibrosing agent is obtained. The coated suture can be air-dried. The spray coating process can be repeated one or more times depending on the specific application. The suture can be dried under vacuum to reduce residual solvent levels. This process will result in the fibrosing agent being adsorbed into the suture as well as being surface associated. In one embodiment, the exposure time of the suture to the solvent would be such that the suture would incur no significant permanent dimensional changes. The fibrosing agent may also be present on the surface of the suture. The amount of surface associated fibrosing agent may be reduced by dipping the coated suture into a solvent for the fibrosing agent or by spraying the coated suture with a solvent for the fibrosing agent.

In the above description the suture can be a suture that has not been modified as well as a suture that has been further modified by coating with a polymer (e.g., parylene), surface treated, surface etching, mechanical smoothing or roughening, or grafting prior to the coating process.

In one embodiment, the fibrosing agent and a polymer are dissolved in a solvent, for both the polymer and the fibrosing agent, and are then spray coated onto the suture.

Fibrosing Agent/Polymer with an Inert Solvent

In one embodiment, the solvent is an inert solvent for a suture such that the solvent does not dissolve the suture to any great extent and is not absorbed by the suture to any great extent. The suture can be spray coated, either partially (for example, coating of the self-retaining region only) or completely, in the fibrosing agent/polymer/solvent solution for a specific period of time. The rate of spraying of the fibrosing agent/solvent solution can be altered (e.g., 0.001 ml per sec to 10 ml per sec) to ensure that a good coating of the fibrosing agent is obtained. The coated suture can be air-dried. The spray coating process can be repeated one or more times depending on the specific application. The suture can be dried under vacuum to reduce residual solvent levels. This process will result in the fibrosing agent/polymer being coated on the surface of the suture.

Fibrosing Agent/Polymer with a Swelling Solvent

In one embodiment, the solvent is one that will not dissolve a suture but will be absorbed by the suture. These solvents can thus swell the suture to some extent. The suture can be spray coated, either partially (for example, coating of the self-retaining region only) or completely, in the fibrosing agent/polymer/solvent solution. The rate of spraying of the fibrosing agent/solvent solution can be altered (e.g., 0.001 ml per sec to 10 ml per sec) to ensure that a good coating of the fibrosing agent is obtained. The coated suture can be air-dried. The spray coating process can be repeated one or more times depending on the specific application. The suture can be dried under vacuum to reduce residual solvent levels. This process will result in the fibrosing agent/polymer being coated onto the surface of the suture as well as the potential for the fibrosing agent being adsorbed into the suture. The fibrosing agent may also be present on the surface of the suture. The amount of surface associated fibrosing agent may be reduced by dipping the coated suture into a solvent for the fibrosing agent or by spraying the coated suture with a solvent for the fibrosing agent.

Fibrosing Agent/Polymer with a Solvent

In one embodiment, the solvent is one that will be absorbed by a suture and that will dissolve the suture. The suture can be spray coated, either partially (for example, coating of the self-retaining region only) or completely, in the fibrosing agent/solvent solution. The rate of spraying of the fibrosing agent/solvent solution can be altered (e.g., 0.001 ml per sec to 10 ml per sec) to ensure that a good coating of the fibrosing agent is obtained. The coated suture can be air-dried. The spray coating process can be repeated one or more times depending on the specific application. The suture can be dried under vacuum to reduce residual solvent levels. In the preferred embodiment, the exposure time of the suture to the solvent would be such that there are not significant permanent dimensional changes to the suture (other than those associated with the coating itself). The fibrosing agent may also be present on the surface of the suture. The amount of surface associated fibrosing agent may be reduced by dipping the coated suture into a solvent for the fibrosing agent or by spraying the coated suture with a solvent for the fibrosing agent.

In the above description the suture can be a suture that has not been modified as well as a suture that has been further modified by coating with a polymer (e.g., parylene), surface treated by plasma treatment, flame treatment, corona treatment, surface oxidation or reduction, surface etching, mechanical smoothing or roughening, or grafting prior to the coating process.

b. Other Exemplary Methods for Combining Fibrosing Agents with Sutures

In certain embodiments, a particulate form of the active agent (e.g., silk, wool, cyanoacrylate particles or chitosan) may be coated onto the suture. In one embodiment, the particulate form may be incorporated into a polymeric carrier (e.g., PLG, PLA, polyurethane, suture coatings as described above). Alternatively, or in addition, particles of the active agent can be applied onto a polymer-coated suture. For example, a suture can be coated with a polymer (e.g., a polyurethane) and then allowed to partially dry such that the surface is still tacky. A particulate form of the fibrosing agent or a fibrosing agent and secondary carrier, such as described above, can then be applied to all or a portion of the tacky coating after which the suture is dried.

In certain embodiments, a suture having a polymeric coating with or without a fibrosing agent can be subjected to a thermal treatment process to soften the coating. A fibrosing agent or a fibrosing agent and secondary carrier then is applied to all or a portion of the softened coating.

Coated sutures may be further coated with an additional composition and/or be treated to alter the release characteristics of the coating composition and/or fibrosing agent. For example, a suture having a fibrosing agent or fibrosing composition incorporated into or coated onto the suture may be further coated with a composition or compound which delays the onset of activity of the fibrosing agent for a period of time after implantation. Protection of a biologically active surface can be achieved by coating the suture surface with an inert molecule that prevents access to the active site through steric hindrance. Representative examples of such compositions or compounds include biologically inert materials such as gelatin, PLGA/MePEG film, PLA, polyurethanes, suture coatings as described above, silicone rubbers, surfactants, lipids, or polyethylene glycol, as well as biologically active materials such as heparin (e.g., to induce coagulation). In one embodiment, the active agent (e.g., poly-L-lysine, fibronectin, chitosan, silk, wool, bleomycin, cyclosporine A, or CTGF) on the suture is top-coated with a physical barrier that does not contain a fibrosing agent. The barrier layer can include non-degradable materials or biodegradable materials such as, e.g., gelatin, PLGA/MePEG film, PLA, PLG, or polyethylene glycol. The barrier layer dissolves slowly or degrades once implanted into the host. As the top layer dissolves or degrades, the active agent becomes exposed to the surrounding tissue and/or can be released from the coating.

Within yet another embodiment, the outer layer of the coated suture, which is capable of inducing an in vivo fibrotic response, is further treated to crosslink or functionalize the outer layer of the coating. Crosslinking of the coating (and/or additional surface modification) can be accomplished using a variety of methods, including, for example, subjecting the coated suture to a plasma treatment process. The degree of crosslinking and nature of the surface modification can be altered by changing the RF power setting, the location with respect to the plasma, the duration of treatment, as well as the gas composition introduced into the plasma chamber.

Protection of a biologically active surface can also be achieved by coating the surface with an inactive form of the fibrosing agent, which is later activated. The fibrosing suture may be activated before, during, or after deployment (e.g., an inactive agent on the suture may be first activated to one that induces or accelerates an in vivo fibrotic reaction).

In one embodiment, the suture can be coated with an inactive form of the fibrosing agent, such as poly-L-lysine, fibronectin, chitosan, silk, wool, bleomycin, cyclosporine A, or CTGF, applied as described herein, which is then activated once the suture is deployed. Activation can be achieved by injecting an activating agent (e.g., an enzyme) or a composition that includes an activating agent into the tissue or area surrounding the suture after deployment of the suture or after the fibrosing agent has been administered to the tissue (via drug delivery catheters or balloons).

In one embodiment, a suture includes a first coating layer that includes a biologically active fibrosing agent, such as poly-L-lysine, fibronectin, or chitosan, bleomycin, silk, wool, cyclosporine A, or CTGF, and a first reactive component. In one embodiment, the first reactive component is capable of reaction with a polyethylene glycol. The coated suture can be further coated with a second composition that includes a second reactive component (e.g., polyethylene glycol) that is capable of reaction with the first reactive component in the first coating layer. The reactive components of the first and second coating layers can be bonded via a condensation reaction through formation of ester bonds. Prior to the deployment of the intra-arterial segment of the suture, an esterase is injected into the treatment site around the outside of the suture, which can cleave the ester linkages, thus allowing the agent to become available to initiate fibrosis.

2. Fibrosing Agent Releasing Profiles

In certain embodiments, fibrosing agents may be released from sutures that comprise fibrosing agents. In other embodiments, the fibrosing agents may be released from carriers that are applied to the surrounding tissue as liquids, gels, pastes, suspensions, microspheres, nanoparticles or other such delivery vehicles. In certain other embodiments, such agents become part of sutures permanently and no fibrosing agents are released from the suture.

In certain embodiments of the present invention, therapeutic compositions coated onto or into, or otherwise attached to, sutures are biocompatible, and release one or more fibrosing agents over a period ranging from several hours, to several days, or over a period of many months. The fibrosing agent that is on, in or near the suture may be released from the composition and/or suture in a time period that may be measured from the time of implantation, which ranges from about less than 1 day to about 180 days. Generally, the release time may also be from about less than 1 day to about 7 days; from 7 days to about 14 days; from 14 days to about 28 days; from 28 days to about 56 days; from 56 days to about 90 days; from 90 days to about 180 days.

The sutures of the present invention may be configured to release the fibrosing agent at one or more phases with similar or different performance (e.g., release) profiles. The fibrosing agent may be made available to the tissue at amounts which may be sustainable, intermittent, or continuous; in one or more phases; and/or rates of delivery; effective to increase or promote any one or more components of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Thus, the release rate may be programmed to impact fibrosis (or scarring) by releasing the fibrosing agent at a time such that at least one of the components of fibrosis is promoted or increased. Moreover, the predetermined release rate may reduce agent loading and/or concentration as well as potentially providing minimal drug washout and thus, increases efficiency of drug effect. In one embodiment, the rate of release may provide a sustainable level of the fibrosing agent to the treatment site. In another embodiment, the rate of release is substantially constant. The rate may decrease and/or increase over time, and it may optionally include a substantial non-release period. The release rate may comprise a plurality of rates. In an embodiment, the plurality of release rates may include rates selected from the group consisting of substantially constant, decreasing, increasing, and substantially non-releasing.

The total amount of fibrosing agent made available on, in or near the suture may be in an amount ranging from about 0.01 μg (micrograms) to about 250 mg (milligrams). Generally, the fibrosing agent may be in the amount ranging from 0.01 μg to about 10 μg; or from 10 μg to about 50 μg, or from 50 μg to 100 μg, or from 100 μg to 1 mg; or from 1 mg to about 10 mg; or from 10 mg to about 100 mg; or from 100 mg to about 250 mg.

The surface amount of fibrosing agent on, in or near the suture may be in an amount ranging from less than 0.01 μg to about 250 μg per mm² of suture surface area. Generally, the fibrosing agent may be in the amount ranging from less than 0.01 μg/mm²; or from 0.01 μg to about 10 μg/mm²; or from 10 μg to about 25 μg/mm²; or from 25 μg to about 250 μg/mm².

In one aspect, “quick release” or “burst” therapeutic compositions are provided that release equal to or greater than 10%, 20%, 25% or 50% (w/v) of a fibrosing agent over a period of 7 to 10 days. Such “quick release” compositions should, within certain embodiments, be capable of releasing therapeutic levels (where applicable) of a desired fibrosing agent. Within other embodiments, “slow release” therapeutic compositions are provided that release less than 1% (w/v) of a fibrosing agent over a period of 7 to 10 days. Within other embodiments therapeutic compositions are provided that release either less than 1% (w/v) of a fibrosing-inducing agent over a period longer than 10 days or do not release the therapeutic composition at all, but maintain the composition for a very long period of time such as for the entire duration of the suture placement in the body.

The amount of fibrosing agent released from the composition and/or suture as a function of time may be determined based on the in vitro release characteristics of the agent from the composition. The in vitro release rate may be determined by placing the fibrosing agent within the composition or suture in an appropriate buffer such as 0.1 M phosphate buffer (pH 7.4)) at 37° C. Samples of the buffer solution are then periodically removed for analysis by either HPLC or by gravimetric means, and the buffer is replaced to avoid any saturation effects.

Based on the in vitro release rates, the release of fibrosing agent per day may range from an amount ranging from about 0.0 μg (micrograms) to about 250 mg (milligrams). Generally, the fibrosing agent that may be released in a day may be in the amount ranging from 0.0 to 0.01 μg; 0.01 μg to about 10 μg; or from 10 μg to about 1 mg; or from 1 mg to about 10 mg; or from 10 mg to about 100 mg; or from 100 mg to about 250 mg. In one embodiment, the fibrosing agent is made available to the susceptible tissue site in a constant but substantially unchanging manner so that the agent remains at the tissue essentially permanently. In another embodiment, the fibrosing agent is made available to the susceptible tissue in a sustained and/or controlled manner that results in increased efficiency and/or efficacy. Further, the release rates may vary during either or both of the initial and subsequent release phases. There may also be additional phase(s) for release of the same substance(s) and/or different substance(s).

3. Sterilization

Further, sutures that comprise fibrosing agents (as well therapeutic compositions useful in making, or using in combination of, the sutures) of the present invention should preferably be have a stable shelf-life for at least several months and capable of being produced and maintained under sterile conditions. The compositions or sutures may be sterile either by preparing them under aseptic environment and/or they may be terminally sterilized using methods available in the art. Many pharmaceuticals/medical devices are manufactured to be sterile and this criterion is defined by the USP XXII <1211>. The term “USP” refers to U.S. Pharmacopeia (see www.usp.org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed aseptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Sterilization may also occur by terminally using gamma radiation or electron beam sterilization methods. A combination of these methods may also be used to prepare the compositions and sutures in the sterile form.

E. Methods for Using Sutures in Combination with Fibrosing Agents

Sutures used in combination with fibrosing agents according to the present application have numerous applications. Exemplary applications are described in detail below. In certain embodiments, sutures themselves comprise fibrosing agents. In certain other embodiments, sutures are not first combined with fibrosing agents or compositions comprising fibrosing agents and then implanted into a host. Instead, fibrosing agents or compositions comprising fibrosing agents are delivered separately to the site where the sutures have been, are being, or are to be implanted. Such delivery may be performed by the use of drug-delivery catheter or by injections or direct applications (e.g., at wound sites).

The hosts on which various applications using sutures in combinations with fibrosis agents are performed may be mammals. In certain embodiments, the hosts are humans. In certain other embodiments, other mammals (including but not limited to a non-human primate, rodent, cat, dog, horse, pig, bovine, sheep, or goat) may be the hosts.

The implantation or insertion of, or the stitching with, a suture (with or without a fibrosing agent) may be performed using the suture itself (e.g., a suture with one or two pointing ends), a needle removably attached to the suture, or an insertion means (e.g., a hollow device in which the suture may be hosted during insertion and subsequently removed to leave the suture inside the host tissue) (see, e.g., U.S. Pat. No. 5,342,376).

For the specific applications described below, any suitable techniques by which a suture is properly implanted or used known in the art may be used. Such techniques include, but are not limited to, alpha suture, zigzag suture, coil suture, “switch back” suture, “finger-trap” suture, Connell suture, everting suture, Halsted suture, horizontal mattress suture, Lembert suture, lock or lock-stitch suture, locking-stitch suture, purse-string suture, subcuticular suture, and vertical mattress suture (see, e.g., Medical Textiles 2004, Advances in Biomedical Textiles and Healthcare Products, Conference Proceedings, IFA/Expo 2004, Oct. 26-27, 2004, Pittsburgh, Pa., pp 262-80). Description of certain exemplary techniques for using self-retaining sutures may be found in U.S. Pat. Nos. 6,773,450, 6241,747, and 6,599,310 and U.S. Patent Application Publication No. 2003/0074023, some of which are briefly provided below.

Any of the aforementioned sutures may be used in combination of a fibrosing agent in various applications according to the present invention. For example, sutures may be biodegradable (or absorbable) or non-biodegradable (or non-absorbable), plain or self-retaining (one-directional or bi-directional). In certain embodiments, the suture may be attached to a needle or another insertion device. In certain other embodiments, the suture may further comprise an anchor member at one end for securing the implantation of the suture in a soft or hard tissue. In certain other embodiments, the suture is a suture connector as described above.

Any of the aforementioned fibrosis-inducing agents and formulations is suitable for use in combination of sutures according to the present invention. In certain embodiments (e.g., the specific applications of the present invention described in detail below), particularly preferred agents include silk, talc, bleomycin, fibronectin, degradable cyanoacrylate, CTGF, chitosan, polylysine, an inflammatory cytokine (e.g., TGFβ, PDGF, VEGF, bFGF, TNFα, NGF, GM-CSF, IGF-α, IL-1, IL-1-β, IL-8, IL-6, and growth hormone), and/or an agent that stimulates cellular proliferation [such as dexamethasone, isotretinoin (13-cis retinoic acid), 17-β-estradiol, estradiol, 1-α-25 dihydroxyvitamin D₃, diethylstibesterol, cyclosporine A, L-NAME, all-trans retinoic acid (ATRA)], and analogues and derivatives of the aforementioned agents.

As sutures are made in a variety of configurations and sizes, the exact dosage administered can vary with the amount injected or the size, surface area and design of the sutures. However, certain principles can be applied in the application of this art. Drug dose can be calculated as a function of dose per area of the surface area of the portion of the suture being coated, total drug dose administered can be measured, and appropriate surface concentrations of active drug can be determined. Regardless of the method of application of the fibrosis-inducing agent in the applications described in detail below, the exemplary fibrosing agents, used alone or in combination, should be administered under the following dosing guidelines:

Utilizing talc as an exemplary fibrosis-inducing agent, whether it is applied using a polymer coating, incorporated into the polymers that make up the suture, or applied without a polymeric carrier, the total dose of talc coated onto the surface of a suture should not exceed 100 mg (range of 0.01 μg to 100 mg). In another embodiment, talc should be applied to a suture surface at a dose of 0.05 μg/mm²-10 μg/mm² of surface area coated. In one embodiment, the total amount of talc released should be in the range of 0.005 μg to 50 mg. In another embodiment, virtually all of the talc is released from the suture. In one embodiment, talc is released from a suture such that fibrosis in the tissue is promoted for a period ranging from several hours to several months. As specific drug delivery vehicles (polymeric and non-polymeric) differ, sutures may release talc at differing rates. The above dosing parameters should be utilized in combination with the release rate of the agent from a suture such that a minimum concentration of talc of 0.01 ng/mg tissue to 1000 μg/mg tissue is delivered to the tissue for the required duration of the effect. For example, in a preferred embodiment talc may be released in effective concentrations for a period ranging from 3-12 months. It should be readily evident given the discussions provided herein that analogues and derivatives of talc (as described previously) with similar functional activity can be utilized for the purposes of this invention; the above dosing parameters are then adjusted according to the relative potency of the analogue or derivative as compared to the parent compound (e.g., a compound twice as potent as talc is administered at half the above parameters, a compound half as potent as talc is administered at twice the above parameters, etc.).

Utilizing silk as an exemplary fibrosis-inducing agent, whether it is applied using a polymer coating, incorporated into the polymers which make up the suture, or applied without a polymeric carrier, the total dose of silk coated onto the surface of a suture should not exceed 100 mg (range of 0.01 μg to 100 mg). In another embodiment, silk should be applied to a suture surface at a dose of 0.05 μg/mm²-10 μg/mm² of surface area coated. In one embodiment, the total amount of silk released should be in the range of 0.005 μg to 50 mg. In another embodiment, virtually all of the silk is released from the suture. As specific (polymeric and non-polymeric) drug delivery vehicles differ, sutures can release silk at differing rates. The above dosing parameters should be utilized in combination with the release rate of the agent from a suture such that a minimum concentration of silk of 0.01 ng/mg tissue to 1000 μg/mg tissue is delivered to the tissue for the required duration of the effect. In one embodiment, silk is released from a suture such that fibrosis in the tissue is promoted for a period ranging from several hours to several months. For example, in a preferred embodiment silk may be released in effective concentrations for a period ranging from 3-12 months. It should be readily evident given the discussions provided herein that analogues and derivatives of silk (as described previously) with similar functional activity can be utilized for the purposes of this invention; the above dosing parameters are then adjusted according to the relative potency of the analogue or derivative as compared to the parent compound (e.g., a compound twice as potent as silk is administered at half the above parameters, a compound half as potent as silk is administered at twice the above parameters, etc.).

Utilizing chitosan as an exemplary fibrosis-inducing agent, whether it is applied using a polymer coating, incorporated into the polymers that make up the suture, or applied without a polymeric carrier, the total dose of chitosan coated onto the surface of a suture should not exceed 100 mg (range of 0.01 μg to 100 mg). In another embodiment, chitosan should be applied to a suture surface at a dose of 0.05 μg/mm²-10 μg/mm² of surface area coated. In one embodiment, the total amount of chitosan released should be in the range of 0.01 μg to 50 mg. In another embodiment, virtually all of the chitosan is released from the suture. As specific (polymeric and non-polymeric) drug delivery vehicles differ, sutures can release chitosan at differing rates. The above dosing parameters should be utilized in combination with the release rate of the agent from a suture such that a minimum concentration of chitosan of 0.01 ng/mg tissue to 1000 μg/mg tissue is delivered to the tissue for the required duration of the effect. In one embodiment, chitosan is released from of a suture such that fibrosis in the tissue is promoted for a period ranging from several hours to several months. For example, in a preferred embodiment chitosan may be released in effective concentrations for a period ranging from 3-12 months. It should be readily evident given the discussions provided herein that analogues and derivatives of chitosan (as described previously) with similar functional activity can be utilized for the purposes of this invention; the above dosing parameters are then adjusted according to the relative potency of the analogue or derivative as compared to the parent compound (e.g., a compound twice as potent as chitosan is administered at half the above parameters, a compound half as potent as chitosan is administered at twice the above parameters, etc.).

Utilizing polylysine as an exemplary fibrosis-inducing agent, whether it is applied using a polymer coating, incorporated into the polymers that make up the suture, or applied without a polymeric carrier, the total dose of polylysine coated onto the surface of a suture should not exceed 100 mg (range of 0.01 μg to 100 mg). In one embodiment, polylysine should be applied to a suture surface at a dose of 0.05 μg/mm²-10 μg/mm² of surface area coated. In one embodiment, the total amount of polylysine released should be in the range of 0.005 μg to 50 mg. In another embodiment, virtually all of the polylysine may be released from the suture. As specific (polymeric and non-polymeric) drug delivery vehicles differ, sutures can release polylysine at differing rates. The above dosing parameters should be utilized in combination with the release rate of the agent from a suture such that a minimum concentration of polylysine of 0.01 ng/mg tissue to 1000 μg/mg tissue is delivered to the tissue for the required duration of the effect. In one embodiment, polylysine is released from a suture such that fibrosis in the tissue is promoted for a period ranging from several hours to several months. For example, in a preferred embodiment polylysine may be released in effective concentrations for a period ranging from 3-12 months. It should be readily evident given the discussions provided herein that analogues and derivatives of polylysine (as described previously) with similar functional activity can be utilized for the purposes of this invention; the above dosing parameters are then adjusted according to the relative potency of the analogue or derivative as compared to the parent compound (e.g., a compound twice as potent as polylysine is administered at half the above parameters, a compound half as potent as polylysine is administered at twice the above parameters, etc.).

Utilizing fibronectin as an exemplary fibrosis-inducing agent, whether it is applied using a polymer coating, incorporated into the polymers that make up the suture, or applied without a polymeric carrier, the total dose of fibronectin coated onto the surface of a suture should not exceed 100 mg (range of 0.01 μg to 100 mg). In another embodiment, fibronectin should be applied to a suture surface at a dose of 0.05 μg/mm²-10 μg/mm² of surface area coated. In one embodiment, the total amount of fibronectin released should be in the range of 0.005 μg to 50 mg. In another embodiment, virtually all of the fibronectin may be released from the suture. As specific (polymeric and non-polymeric) drug delivery vehicles differ, sutures can release fibronectin at differing rates. The above dosing parameters should be utilized in combination with the release rate of the agent from a suture such that a minimum concentration of fibronectin 0.01 ng/mg tissue to 1000 μg/mg tissue is delivered to the tissue for the required duration of the effect. In one embodiment, fibronectin is released from a suture such that fibrosis in the tissue is promoted for a period ranging from several hours to several months. For example, in a preferred embodiment fibronectin may be released in effective concentrations for a period ranging from 3-12 months. It should be readily evident given the discussions provided herein that analogues and derivatives of fibronectin (as described previously) with similar functional activity can be utilized for the purposes of this invention; the above dosing parameters are then adjusted according to the relative potency of the analogue or derivative as compared to the parent compound (e.g., a compound twice as potent as fibronectin is administered at half the above parameters, a compound half as potent as fibronectin is administered at twice the above parameters, etc.).

Utilizing bleomycin as an exemplary fibrosis-inducing agent, whether it is applied using a polymer coating, incorporated into the polymers that make up the suture, or applied without a polymeric carrier, the total dose of bleomycin coated onto the surface of a suture should not exceed 100 mg (range of 0.01 μg to 100 mg). In one embodiment, bleomycin should be applied to a suture surface at a dose of 0.005 μg/mm²-10 μg/mm² of surface area coated. In one embodiment, the total amount of bleomycin released should be in the range of 0.005 μg to 50 mg. In another embodiment, virtually all of the bleomycin may be released from the suture. As specific (polymeric and non-polymeric) drug delivery vehicles differ, sutures can release bleomycin at differing rates. The above dosing parameters should be utilized in combination with the release rate of the agent from a suture such that a minimum concentration of bleomycin of 0.001 nM to 1000 μM is delivered to the tissue for the required duration of therapeutic effect. In one embodiment, bleomycin is released from a suture such that fibrosis in the tissue is promoted for a period ranging from several hours to several months. For example, in a preferred embodiment bleomycin may be released in effective concentrations for a period ranging from 3-12 months. It should be readily evident given the discussions provided herein that analogues and derivatives of bleomycin (as described previously) with similar functional activity can be utilized for the purposes of this invention; the above dosing parameters are then adjusted according to the relative potency of the analogue or derivative as compared to the parent compound (e.g., a compound twice as potent as bleomycin is administered at half the above parameters, a compound half as potent as bleomycin is administered at twice the above parameters, etc.).

Utilizing CTGF as an exemplary fibrosis-inducing agent, whether it is applied using a polymer coating, incorporated into the polymers that make up the suture, or applied without a polymeric carrier, the total dose of CTGF coated onto the surface of a suture should not exceed 100 mg (range of 0.01 μg to 100 mg). In one embodiment, CTGF should be applied to a suture surface at a dose of 0.005 μg/mm²-10 μg/mm² of surface area coated. In one embodiment, the total amount of CTGF released should be in the range of 0.005 μg to 50 mg. In another embodiment, virtually all of the CTGF may be released from the suture. As specific (polymeric and non-polymeric) drug delivery vehicles differ, sutures can release CTGF at differing rates. The above dosing parameters should be utilized in combination with the release rate of the agent from a suture such that a minimum concentration of CTGF of 0.001 ng/mg tissue to 1000 μg/mg tissue is delivered to the tissue for the required duration of the effect. In one embodiment, CTGF is released from a suture such that fibrosis in the tissue is promoted for a period ranging from several hours to several months. For example, in a preferred embodiment CTGF may be released in effective concentrations for a period ranging from 3-12 months. It should be readily evident given the discussions provided herein that analogues and derivatives of CTGF (as described previously) with similar functional activity can be utilized for the purposes of this invention; the above dosing parameters are then adjusted according to the relative potency of the analogue or derivative as compared to the parent compound (e.g., a compound twice as potent as CTGF is administered at half the above parameters, a compound half as potent as CTGF is administered at twice the above parameters, etc.).

Optionally, the suture may alone or additionally comprise an inflammatory cytokine (e.g., TGFβ, PDGF, VEGF, bFGF, TNFα, NGF, GM-CSF, IGF-a, IL-1, IL-1-β, IL-8, IL-6, and growth hormone) or an analogue or derivative thereof.

Inflammatory cytokines are to be used in formulations at concentrations that range from 0.0001 μg/ml to approximately 20 mg/ml depending on the specific clinical application, formulation type (e.g., gel, liquid, solid, semi-solid), formulation chemistry, duration of required application, type of suture interface and formulation volume and or surface area coverage required. Preferably, the inflammatory cytokine is released in effective concentrations for a period ranging from 1-180 days. The total dose for a single application is typically not to exceed 100 mg (range of 0.0001 μg to 100 mg); preferred 0.001 μg to 50 mg. When used as a suture coating, the dose is per unit area of 0.0001 μg-500 μg per mm²; with a preferred dose of 0.001 μg/mm²-200 μg/mm². Minimum concentration of 10⁻¹⁰-10⁻⁴ g/ml of inflammatory cytokine is to be maintained on the suture surface for the required duration of therapeutic effect.

Furthermore, the suture may alone or additionally comprise an agent that stimulates cellular proliferation. Examples include: dexamethasone, isotretinoin (13-cis retinoic acid), 17-β-estradiol, estradiol, 1-α-25 dihydroxyvitamin D₃, diethylstibesterol, cyclosporine A, L-NAME, all-trans retinoic acid (ATRA), and analogues and derivatives thereof. Doses used are those concentrations that are demonstrated to stimulate cell proliferation. The proliferative agents are to be used in formulations at concentrations that range from 0.1 ng/ml to 25 mg/ml depending on the specific clinical application, formulation type (e.g., gel, liquid, solid, semi-solid), formulation chemistry, duration of required application, type of suture interface and formulation volume and or surface area coverage required. Preferably, the proliferative agent is released in effective concentrations for a period ranging from 1-180 days. The total dose for a single application is typically not to exceed 500 mg (range of 0.0001 μg to 200 mg); preferred 0.001 μg to 100 mg. When used as a suture coating, the dose is per unit area of 0.00001 μg-500 μgper mm²; with a preferred dose of 0.0001 μg/mm²-200 μg/mm². Minimum concentration of 10⁻¹¹-10⁻⁶ M of proliferative agent is to be maintained on the suture surface for the required duration of therapeutic effect.

It should be readily evident to one of skill in the art that any of the previously described fibrosis inducing agents, or derivatives and analogues thereof, can be utilized to create variations of the above compositions without deviating from the spirit and scope of the invention. It should also be apparent that the agent can be utilized in a composition with or without polymer carrier and that altering the carrier does not deviate from the scope of this invention.

1. Tissue Repositioning

In one aspect, the present invention provides a method for repositioning tissue using a suture that comprises a fibrosing agent. Such method comprises: implanting a suture into a tissue in need thereof, and moving the suture so that the tissue into which the suture is implanted is repositioned, wherein the suture comprises a fibrosing agent that induces or stimulates a fibrotic response between the suture and the tissue in which the suture is implanted. In a further embodiment, the suture comprising a fibrosing agent induces or stimulates a fibrotic response within the tissue in which the suture is implanted.

The tissue that may be repositioned may be any tissue in the body for which repositioning (e.g., lifting) is desirable. Such tissue includes excess or sagging tissues in the face, brow, jowls, neck, chin, breast, thigh, arms, abdomen, and buttocks.

In certain embodiments, the sutures useful in this application are nondegradable self-retaining sutures (e.g., CONTOUR THREAD®). In some embodiments, the sutures may be degradable self-retaining sutures; while in certain other embodiments, the sutures are not self-retaining but possess other mechanical tissue fixation properties. In certain embodiments, suture connectors (with or without barbs or other retainers) or suture anchors (with or without barbs or other retainers) may be used. In the embodiments where self-retaining sutures are used, such sutures may be uni-directional or bi-directional. Sutures may be inserted into the tissue in need thereof via their own pointing ends, needles or other insertion means.

Certain specific applications of the tissue repositioning method according to the present invention are described in more detail below.

a. Facial Repositioning Procedures (Facelifts)

As the aging process progresses, the skin's connective tissue thins and collagen and elastic fibers begin to break down. These changes in connective tissue are manifested in underlying support structures as sagging, deep creases, folds and wrinkles in the skin. In the face, brow, neck and chin, aging results in increased laxity and redundancy in the cervicofacial skin, atrophy of the subcutaneous fat, attenuation of the subcutaneous musculoaponeurotic system and resorption of alveolar bone. Cosmetically this results in the creation of an obtuse cervicomental angle, anterior platysmal banding, jowling with loss of the inferior mandibular contour, inferior displacement of cheek fat with diminution of the malar eminence and ptosis (sagging) of the lateral brow.

Invasive corrective procedures involve surgically separating the redundant skin from the underlying tissue under general anaesthesia, raising it, and affixing it in a different (more cosmetically appealing) anatomical location (a “facelift”). With the advent of the self-retaining suture, it became possible to perform a minimally invasive facelift procedure performed with the patient awake under local anaesthesia. In this procedure, a self-retaining suture is placed between the supporting tissue and the subcutaneous tissue such that the superficial tissue can be elevated over the barbs, adjusted to the desired anatomical location and affixed in place. The result is repositioning of the superficial tissues over the deeper tissues into a more vertical and youthful position.

Self-retaining sutures can be broadly categorized into two types: unidirectional and bidirectional. In some embodiments, the procedures may be performed with a one-way self-retaining suture having an elongated body, a hard pointed distal end for penetrating the tissue, and a plurality of retainers extending from the surface of the body, wherein the retainers allow the entire suture to move unidirectionally through the tissue in the direction of movement of the single insertion end. In other embodiments, the facelift procedures are performed with a two-way (bidirectional) self-retaining suture having an elongated body, two hard pointed distal ends for penetrating the tissue, and a plurality of retainers extending from the surface of the body. Retainers on one portion of the body of the suture permit movement of that portion of the suture through the tissue in a direction of movement of that end of the suture, and prevent movement of that portion of the suture in an opposite direction to the direction of movement of that end. Retainers on the other portion of the body of the suture permit movement of that portion of the suture through the tissue in the direction of movement of that end of the suture, and prevent movement of that portion of the suture in an opposite direction opposite to the direction of movement of that end.

In a typical minimally invasive facelift procedure, an incison is made behind the hairline. The first end of the self-retaining suture is inserted through the incision and pushed through subepidermal tissue along a marked path until it exits from the skin at a predetermined point. This may be repeated several times in several different directions and tissue plains (placing 8-12 threads or more) depending upon the extent of the procedure and the desired end effect. In some embodiments, an insertion device may be used to insert the self-retaining suture. Such an insertion device may be tubular, with a circular or some other cross-sectional shape, and has openings at both ends. The device may be referred to as a needle, but any sharp pointed end, including a hard end of the suture itself may be used for insertion.

Once inserted under the skin, the retainers open up in an “umbrella-like” fashion to form a support structure. The first end of the suture is gripped and pulled to draw the first portion of the suture through the soft tissue until a length of the first self-retaining portion of the suture is left in the soft tissue between the point of insertion and the exit point. For unidirectional self-retaining sutures, the end of the suture at the incision site is mechanically tied to the surrounding tissue; this is not necessary for bidirectional self-retaining sutures. The tissue is then manually and progressively lifted and positioned over the scaffolding provided by the retainers until the desired amount of lift is achieved. In effect, the self-retaining suture functions as a support structure onto which the dermal tissue is lifted and affixed until it is in a more vertical and/or youthful position.

Despite this progress, the self-retaining sutures are not without their limitations. Often their hold is insufficient and tissue slippage occurs and impairs wound healing. In some cases, the tissue never becomes completely adherent to the suture or to the adjacent tissues leading to drooping of the facial tissues and a gradual loss of efficacy. For tissue repositioning procedures, having the elevated tissue permanently adhere to the underlying tissue against which it is placed will not only enhance the durability of the procedure (typically the procedure is only effective for 3-5 years) and shorten the period during which the skin cannot be manipulated, but will allow the use of fully degradable sutures, since ongoing physical tissue support would not be required.

By employing the embodiments of the present invention, it is possible to increase the efficacy, durability and patient satisfaction associated with this procedure. By applying a fibrosis-inducing agent on, or around, the self-retaining suture (such as the CONTOUR THREAD™) as described previously, the adjacent tissues will be stimulated to adhere not just to the suture material, but to each other. As such, the lifted dermal tissue will become permanently engrafted onto the underlying deep tissue through the creation of fibrous connective tissue between the two. This will result in a more permanent fixation and gradually eliminate the need for the support provided by the suture; thereby not only eliminating problems associated with suture stretching and material fatigue, but allowing the use of degradable suture material.

Accordingly, in certain embodiments, the present invention provides a method for facelifting comprising: implanting a suture that comprises a fibrosing agent into the subepidermal tissue of the face, and moving the suture so that the dermal tissue of the face is uplifted.

b. Brow Repositioning Procedures (Browlifts)

Sutures (e.g., self-retaining sutures) that comprise fibrosing agents, or used in combination with fibrosing agents or compositions according to the present invention, may also be used for a variety of other facial procedures such as treating ptotic brows, cheeks, necks and elevating and providing the appearance of fullness in the lips. For example, a browlift may be achieved in a patient via linear placement of self-retaining sutures on both sides of the forehead, without any incision. The self-retaining suture may be inserted vertically through a minute puncture wound, immediately inferior to the hairline on each side of the forehead, and extended superiorly from the junction of the middle and lateral third of the eyebrow. The suture depth may be midway between the skin and frontalis muscle. After the tissue is massaged over the suture into the desired position, the brow may be elevated several milimeters bilaterally.

c. Thigh Tissue Repositioning

Sutures (e.g., self-retaining sutures) that comprise fibrosing agents, or used in combination with fibrosing agents or compositions according to the present invention, may be also used for thigh tissue repositioning. For repositioning or lifting of thigh tissue, the insertion point is generally at the inguinal crease. The first end of the suture is pushed cranially through subepidermal tissue until the first end of the suture extends out of the tissue. The second end of the suture is pushed caudally through subepidermal tissue until the second end of the suture extends out of the tissue on the thigh. The tissues are manually grouped and repositioned to yield the desired amount of lift.

d. Breast Tissue Repositioning (Breastlifts)

Sutures (e.g., self-retaining sutures) that comprise fibrosing agents, or used in combination with fibrosing agents or compositions according to the present invention, may be also used for the minimally invasive treatment of ptosis (sagging) of the breasts. For repositioning or lifting of breast tissue, the insertion point may be at the upper aspect of the breast curvature, and the first end of the suture is pushed through subcutaneous tissue, dermal tissue, and the pectoralis muscle until extending out of the tissue at an exit point on the upper portion of the breast. The second end of the suture is pushed caudally through fibrous and fatty tissues until the second end of the suture extends out of the tissue at an exit point along the anterior aspect or the lower curvature of the breast. The tissue is manually grouped and repositioned to yield the desired amount of lift.

2. Wound Closure

It is well known that sutures have long been the material of choice for closing both deep and superficial wounds. Although a wide variety of sutures and suturing techniques exist, the majority involves piercing the tissue on either side of the wound via a needle such that the suture bridges gap between tissues and forms an open loop. The wound is manually pulled together by applying tension on the suture such that the adjacent tissues are approximated, and the process is concluded when the physician ties a knot to complete the suture loop. The procedure is repeated until enough “stitches” are in place such that the edges of the wound are in close enough contact for normal healing to take place. The more tension required to hold the wound together, the larger the diameter of the suture material. A large number of nondegradable and degradable sutures in numerous lengths, diameters, compositions, needle configurations and filament types have been prepared to accomplish this purpose. Almost as many different suturing techniques exist, including interrupted and running suturing, all with the goal of bringing the tissues into close approximation.

However, it has long been recognized that the integrity of the suture material and the knot are essential to successful tissue approximation and wound healing. If the tissue slips over the suture, if the suture pulls through the tissue, if the suture material stretches or breaks, or if the knot loosens or becomes untied, tension is lost and the approximated tissues can separate. This can lead to a variety of clinical complications such as wound dehiscence, excessive inflammatory response, procedural failure (particularly devastating when the suture is holding vital structures together), infection, excessive scarring or adhesions, and poor cosmetic results for superficial wounds. In attempt to reduce this risk, efforts have been made to overcome these deficiencies with “knotless” sutures that can anchor in place without the requirement of knot tying.

As described in detail above, self-adhering sutures have been produced in a variety of designs and configurations that utilize a “barb” structure that can be unidirectional or bidirectional in nature. The tissue through which the suture passes becomes “hooked” on the retainers and anchored to the suture material thus preventing the tissue from slipping relative to the suture and (in some cases) providing enough support that knot tying is not required. Despite this progress, the self-retaining sutures are not without their limitations. Often their hold is insufficient and tissue slippage occurs and impairs wound healing. In some cases, the tissue never becomes completely adherent to the suture or to the adjacent tissues leading to a gradual loss of efficacy.

Sutures (e.g., self-retaining sutures) that comprise fibrosing agents, or are used in combination with fibrosing agents or compositions according to the present invention, may be used for closing a wound. By employing the embodiments of the present invention, it is possible to increase the efficacy, durability, efficiency and longevity of a sutured wound closure. By applying a fibrosis-inducing agent on, or around, the self-retaining suture (such as, for example, the CONTOUR THREAD™) as described previously, the adjacent edges of the wound will be stimulated to adhere not just to the suture material, but to each other. As such, the fibrosing agents promote permanent grafting of the wound edges to each other through the creation of fibrous connective tissue between the two. This will result in a more permanent fixation and gradually eliminate the need for the support provided by the suture; thereby not only eliminating problems associated with suture stretching and material fatigue, but also allowing the use of degradable suture material (even in higher tension wounds). It may also be possible to bring together tissues where knot tying is difficult (such as deep wounds or endoscopic procedures).

Accordingly, in one aspect, the present invention provides a method for closing a wound comprising: joining two surfaces or edges of a wound together with a suture that comprises a fibrosing agent, wherein the fibrosing agent induces or stimulates a fibrotic response between the suture and the tissue in contact with the suture.

In using the above wound closure method of the present invention, the user will generally select a biodegradable or nondegradable suture that comprises a fibrosing agent of sufficient length and strength. In some emboments, the suture may be a one-way self-retaining suture having an elongated body, a hard pointed distal end for penetrating the tissue, and a plurality of retainers extending from the surface of the body, wherein the retainers allow the entire suture to move through the tissue in the direction of movement of the single insertion end. In other embodiments, the suture may be a two-way self-retaining suture having an elongated body, two hard pointed distal ends for penetrating the tissue, and a plurality of retainers extending from the surface of the body. Retainers on one portion of the body of the suture permit movement of that portion of the suture through the tissue in a direction of movement of that end of the suture, and prevent movement of that portion of the suture in a direction opposite to the direction of movement of that end. Retainers on the other portion of the body of the suture permit movement of that portion of the suture through the tissue in the direction of movement of that end of the suture, and prevent movement of that portion of the suture in a direction opposite to the direction of movement of that end.

In certain embodiments, when using a fibrosis-inducing two-way self-retaining suture and a needle as an insertion device, the user inserts the needle having the first end of the suture into the tissue on a first side of the wound and laterally spaced from the face of the wound, near the midpoint of the length of the wound. The user advances the needle along a selected substantially straight path through the tissue to extend out of the tissue at a selected point on the first face of the wound and subsequently penetrating a point on a second face of the opposite side of the wound. The user continues to advance the needle through the tissue until the point of the needle emerges from the tissue at a distal end of the selected path at an exit point on the second side of the wound and laterally spaced from the second face of the wound. Gripping the exposed portion of the needle and pulling the needle out of the tissue, the user draws the suture through the tissue until the retainers on the second end of the suture engage the surface of the wound at the insertion point, preventing further advancement of the suture through this part of the tissue. The user pushes adjacent sides of the wound together at the point where the suture passes through the two faces of the wound.

The user then inserts the first end of the suture (with the needle as an insertion device) into the exit hole and advances the suture through the tissue and the two faces of the wound along a path that is at an angle to that of the first insertion. The needle is then advanced to exit from the tissue at a point on the first side of the wound. Again the user grips and pulls the needle out of the tissue. This process is repeated with the suture forming a zigzag pattern. The number of passes of the needle with the first end of the suture is chosen in accordance with the size of the wound and the strength required to hold the wound closed. The sides of the wound may be pushed together with each passage of the suture. When complete, the length of the first end of the suture extending out of the tissue is cut off and discarded. This process is repeated with the second needle carrying the second end of the suture, beginning with insertion of the needle in the same insertion point as that of the needle with the first end of the suture. A similar zigzag pattern of the suture is formed toward to other end of the wound, thereby closing the other half of the wound.

In certain embodiments, a wound may be closed using fibrosis-inducing one-way sutures. In certain embodiments of a procedure for closing a wound or a surgical incision, the one-way sutures are passed through the tissue at each of the opposed faces of the wound, forming suture pairs in which trailing ends of the sutures are positioned generally in alignment at opposite sides of the wound. On insertion of each suture, the needle is pushed to extend out of the tissue at a point laterally remote from the wound, then the needle is pulled out to draw the suture to the desired position, and the suture is severed from the needle. The number of suture pairs is chosen in accordance with the size of the wound and the strength required to hold the wound closed. Once all the sutures are in place, the wound is closed (as by holding or clamping), and the ends of each suture pair is secured together, for example, by heat bonding or surgical knots.

A self-retaining suture that comprises a fibrosing agent may be used to close an axial wound in a blood vessel, such as an artery or a vein, by insertion of the suture with an insertion device. The wound may be, for example, a puncture of an artery, which may occur as the result of introduction and removal of a catheter. The procedure may or may not involve penetration of the vessel. In the absence of penetration of the vessel, the suture is inserted through the tissue from one side of the wound, through the two faces of the wound, and then pushed into the tissue on the other side of the wound. After withdrawal of the insertion device, the two faces of the wound are advanced together. As noted, the suture may be inserted with the device in such a manner that it penetrates into the vessel. In this case, the suture is inserted with the device through the tissue, through the vessel wall on one side of the wound, into the interior of the vessel, through the vessel wall on the other side of the wound, and then into the tissue on the other side of the wound. The insertion device is removed by pulling on its trailing end, leaving the suture in place. A suture for use in this procedure may be designed to have no retainers on the portion of the suture that is positioned within the vessel. This may limit the possibility of blood coagulating on or near the surface of the suture.

In certain embodiments, a suture that comprises a fibrosing agent may be used to close a wound resulting from surgical extraction of a broken or impacted tooth. A suture having an attached surgical needle may be used according to any of the suturing methods described above. The suture is inserted from one side through the faces of the wound, and the tissue is then advanced to secure the faces together and is held in place by the suture. The wound may have multiple faces that must be drawn together. Closure of the wound may be performed by using either multiple short sutures or a single suture. The suturing pattern may include a zigzag, wavelike, or coil type of stitch.

In other embodiments, the present invention also provides a method for closing a wound comprising: infiltrating a wound with a fibrosing agent or a composition comprising a fibrosing agent, and joining two surfaces or edges of the wound together with a suture, wherein the fibrosing agent induces or stimulates a fibrotic response between the suture and the tissue in contact with the suture.

Wounds or incisions for which the sutures and procedures described herein may be used may be external (e.g., skin wound) and/or internal, which may be resulted from an injury, a surgery, a disease, or even the process of suturing a wound site (e.g., puncture of skin by a suturing needle). For example, the sutures and procedures may be used, following vascular surgery or a Caesarean section.

In certain embodiments, the sutures useful in this application are nondegradable self-retaining sutures. In some embodiments, the sutures may be degradable self-retaining sutures; while in certain other embodiments, the sutures are not self-retaining but possess other mechanical tissue fixation properties. In certain embodiments, suture connectors (with or without barbs) or suture anchors (with or without barbs) may be used. In the embodiments where self-retaining sutures are used, such sutures may be uni-directional or bi-directional. Sutures may be inserted into the tissue in need thereof via their own pointing ends, needles or other insertion means.

The fibrosing agent useful in this application may be any one described above. In one embodiment, the fibrosing agent is present in an amount effective to facilitate or accelerate healing of a wound.

3. Other Selected Uses

In addition to tissue reposition and wound closure, the present invention also provides the following exemplary applications:

a. Connecting Severed Ends of a Tendon

In one aspect, the present invention provides a method for contacting severed ends of a tendon, comprising joining the severed ends of the tendon together using a suture that comprises a fibrosing agent.

Tendon repairs after a complete or partial laceration are difficult to repair using strandard suttuing technique because necessary suture techniques are challenging and because the resulting knots are obstructional in a confined space.

In certain embodiments, a two-way self-retaining suture of appropriate length that comprises a fibrosing agent is selected. The ends of the suture may comprise a straight or curved surgical needle. One end of the suture is inserted into a severed end of the tendon. The needle is pushed through the tendon along a selected curvilinear path until the end of the suture exits from the periphery of the tendon at a point appropriately spaced longitudinally from the severed end of the tendon. This process is repeated with the second end of the suture at the other severed end of the tendon. Similarly, the process is repeated with a second suture, with each end of the suture inserted into a severed end of the tendon. An end of a suture is then inserted back into the tendon near the respective exit points and is further passed through the tendon in a curvilinear path until it again exits from the tendon at a point further removed from the severed end of the tendon. This is repeated with each end of each suture. This is further repeated multiple times, with each suture forming a “wave-like” pattern extending away from the severed ends of the tendon. The tendon ends are brought together by pushing the ends along the sutures while maintaining tension on the free ends of the sutures. The retainers maintain the sutures in place and resist movement of the tendon ends away from each other. Once complete, the suture ends, including the needles, are removed.

In certain other embodiments, a self-retaining suture that comprises a fibrosing agent may be used to repair a partial or complete laceration tear with or without the addition of some traditional sutures, depending on the site of laceration and judgment of the surgeon. The self-retaining suture could be fixed proximally on the tendon with a knot, and the self-retaining segment run intra-tendon substance from the proximal tendon, across the laceration, into the distal aspect of the lacerated tendon with final knot fixation on the tendon distally. The surgeon has the option to fix the laceration by running a bidirectional self-retaining suture intra-tendon substance from the proximal tendon, across the laceration and into the distal tendon. The suture exits the tendon distal to the laceration, the surgeon then adjusts the tendon on the self-retaining suture to eliminate the gap in the laceration. The surgeon can then fixate the distal end of the self-retaining suture with a knot or cut the excess self-retaining suture opting to use no knots. The proximal entry point and distal exit points of the suture are such that and adequate amount of self-retaining suture is within the tendon substance to hold the tendon laceration in place.

It is understood that any suitable method known in the art for inserting a suture into the severed ends of a tendon to hold the ends of the tendon together may be used. It is further understood that additional sutures may be used. The number of sutures depends on the size and length of the tendon to be repaired.

b. Joining Tendon or Ligament to Bone Using a Suture Anchor

In one aspect, the present invention provide a method for attaching a tendon to a bone, comprising: inserting into the bone a suture anchor that comprises an anchor member and one or more sutures attached to the anchor member, and connecting the end of the tendon to the bone using the one or more sutures, wherein the one or more sutures comprise a fibrosing agent.

Tendons and ligaments provide high tensile strength connection within the body. Tendons connect muscle to bone, while ligaments connect bone to bone or cartilage to bone. Through injury or damage the attachment of a tendon or ligament to bone may become severed or torn. Repair or reattachment of such a severed or torn attachment requires a surgical procedure that provides a connection having a strong attachment to the bone and high tensile strength. Use of a suture anchor that comprises a fibrosing agent according to the present invention is an advantageous means for adjoining tissue to bone or to other tissues, providing both the strong attachment to the bone and a high tensile strength connection. A suture anchor may be used, for example, to reattach an Achilles tendon that has been torn or severed from the heel bone in the foot.

A suture anchor for use in attaching a tendon or ligament to a bone comprises an anchor member, which is inserted into the bone. The anchor member may be inserted into the bone by being driven, such as by being tapped with a hammer. Alternatively, where the anchor element is threaded, it may be rotated to screw it into the bone. One or more self-retaining sutures that comprise a fibrosing agent are mounted to the end of the anchor member and extend outwardly therefrom. Each suture may be mounted to the anchor at any position along the length of the suture and may have any design described elsewhere herein. For example, a suture may have one or two pointed ends, that is, having a one-way unidirectional or a two-way bidirectional configuration. The ends of the sutures may have straight or curved surgical needles attached to assist in inserting the ends into the tissue.

Once the anchor member has been inserted into the bone, the pointed or needled end or ends of each suture is then inserted through tendon or ligament needing to be affixed to the bone. The retainers on the suture are configured, as described elsewhere herein, to allow insertion of the pointed end or ends of the suture into the tendon or ligament, but to resist withdrawal of the suture from the tendon or ligament. Once the sutures are inserted through the tissue at appropriate locations, the tissue is pushed toward the anchor member to affix it tightly against the bone. Multiple suture anchors may be mounted in the bone as necessary to securely fix the tissue against the bone.

In one embodiment, a torn or severed Achilles tendon may be reattached to the bone of the heel. A fibrosis-inducing suture anchor having sutures of appropriate length is selected. The suture ends may be, but need not necessarily be, surgical needles. The suture anchor is inserted into the heel bone. A first end, or first surgical needle, of a suture is inserted into the free end of and through a length of the Achilles tendon until exiting from an appropriate point on the surface of the tendon. This process is repeated with the second end, or second surgical needle, of a suture, beginning with insertion into the free end of the tendon. The process for further inserting the sutures into the tendon to provide a connection of adequate strength between the tendon and the bone may follow that described above for connecting the two ends of a severed tendon. It is understood that any suitable method known in the art for inserting a suture into the severed ends of a tendon to securely hold the ends of the tendon together may be used for connecting tendon to bone. It is further understood that additional sutures may be used.

Description of certain exemplary techniques for using suture anchors may be found in U.S. Pat. No. 6,773,450. The suture anchor is useful in endoscopic and arthroscopic procedures and microsurgery.

This method is not limited to use for attaching tendons or ligaments to bone, but may similarly be used to affix any tissue to another tissue, where use of an anchor member may be advantageous and thus may be employed in many of the uses for self-retaining sutures described elsewhere herein.

c. Nissen Fundoplication Procedure

In one aspect, the present invneiton provide a method for performing a Nissen fundoplication procedure. Such a method comprises joining portions of the stomach together using a suture that comprises a fibrosing agent.

Joining and holding portions of the stomach to each other in performing the Nissen fundoplication procedure requires grasping the fundus of the stomach at a proximal location and pulling the fundus around the esophagus, wrapping the fundus around the esophagus one time, and attaching the proximal stomach to an apposing portion of the stomach. Any suture techniques appropriate for performing the Nissen fundoplication procedure known in the art may be used. For instance, U.S. Patent Application Publication No. 2003/0074023 describes several techniques for using self-retaining sutures with or without an insertion device in performing the Nissen fundoplication procedure. For example, in one embodiment, a fibrosis-inducing self-retaining suture is inserted into the tissue of the proximal stomach until the first end of the suture extends out of the tissue at an exit point on the exterior of the stomach. The first end of the suture is pulled to draw the first portion of the suture through the stomach tissue until the second axial location is proximate to the pont of insertion of the first end of the suture. A length of the first portion of the suture is left in the tissue between the point of insertion and the exit point of the first end. The proximal stomach is gripped and the fundus is wrapped around the esophagus until the proximal stomach contacts an apposing portion of stomach. The second pointed end of the suture is inserted into tissue of the apposing stomach, and the second end of the suture is pushed through the stomach tissue until the second end of the suture extends out of the tissue at an exit point on the exterior of the stomach. The second end of the suture is pulled to draw the second portion through the tissue until the second axial location is proximate to the point of insertion of the second end of the suture and a length of the second portion of the suture is left in the tissue between the point of insertion and the exit point of the second end.

In another exemplary embodiment of the Nissen fundoplication method, an insertion device is used. First, the fundus is wrapped around the esophagus to form a junction with the apposing portions of stomach. Then the first pointed end of the suture and leading end of the insertion device are inserted into stomach tissue at a point laterally spaced from the junction and on a first side of the junction. The first end of the suture and leading end of the insertion device are pushed through the first side of stomach tissue and penetrate the stomach tissue on a second side of the junction until the portion of the suture between the first and second axial locations is proximate to the junction. The insertion device is removed by gripping and pulling the trailing end, leaving the suture in place.

d. Laparoscopic Insertion of Sutures

In one aspect, the present invention provides a method for laparoscopic insertion of a suture into an abdominal cavity. Such a method comprises: placing a suture that comprises a fibrosing agent in a laparoscopic insertion device, inserting the insertion device having the suture through an abdominal wall into the abdominal cavity, and withdrawing the insertion device. The use of fibrosis-inducing sutures has the advantage of often not requiring knot tying; a difficult task to perform laproscopically.

In one embodiment, the suture that comprises a fibrosing agent is a self-retaining suture and the insertion device for the suture includes a laparoscopic suturing tool. In such an embodiment, the first pointed end of the suture and the leading end of the laparoscopic insertion device may be inserted through an entry point in the skin, and then through the fat, fascia, muscle and peritoneum into the abdominal cavity. The first end of the suture and leading end of the insertion device are pushed into the tissue in the abdominal cavity, and the insertion device is pulled at the trailing end to remove the insertion device. The retainers on the suture are directed to resist withdrawal of the suture. The suture is thus left in place as inserted when the insertion tool is withdrawn.

e. Stabilizing an Internal Structure

In one aspect, the present invention provides a method for an internal structure within the body. Such a method comprises: placing a suture that comprises a fibrosing agent into a laparoscopic insertion device, inserting the insertion device with the suture through an abdominal wall, further inserting the insertion device with the suture into the internal struction in need of stabilization, and withdrawing the insertion device. An end portion of the suture remains attached to the internal structure requiring stabilization and another end portion of the suture remains attached to the abdominal wall, thus stabilizing the internal structure.

In one embodiment, the suture used is a two-way self-retaining suture comprising a fibrosing agent, which may be inserted laparoscopically. For example, a laparoscopic device may be used in stabilizing a segment of intestine in advance of performing an intestinal anastomosis, wherein a segment of intestine is surgically connected to another segment of intestine. A laparoscopic grasping tool is first inserted from the exterior of the body to hold the intestinal structure in position while stabilization by suturing is performed. Once the intestine is held in position, an insertion device with the self-retaining suture is used to laparoscopically insert the suture. The insertion device is passed into the wall of the intestine. The device is then withdrawn leaving one end of the suture in place within the intestinal wall, the retainers preventing withdrawal of the suture. The other end of the suture extends through the abdominal wall, where the retainers hold the suture in place. The intestinal structure may be positioned and is then stabilized by leaving the inserted suture in place in the intestinal wall and the abdominal wall during the surgical preparation of the anastomosis. In performing this stabilization, the lead end of the suture may optionally be extended through the intestinal wall and back into the abdominal wall, such that the intestinal structure is stabilized by a suture attached at both ends to the abdominal wall.

Similarly, the fibrosis-inducing self-retaining sutures may be inserted laparoscopically for bowel surgery. The bowel structure may be positioned and then stabilized by leaving the inserted suture in place in the bowel tissue and the abdominal wall.

It is understood that one skilled in the art could similarly use a one-way suture in these procedures.

f. Closure for a Cystostomy Insertion

In one aspect, the present invention provides a method for closing a cytostomy insertion, comprising joining two surfaces of the cytostomy insertion using a suture that comprises a fibrosing agent.

A cystostomy is a surgically created opening through the abdomen into the urinary bladder. Such a procedure may be performed, for example, when it is necessary to insert a catheter for drainage of urine into a collection device. Upon removal of the catheter, the cystostomy incision must be closed. For closure of the incision, a suture that comprises a fibrosing agent may be inserted laparoscopically. For example, the first and second ends of the suture are inserted in the muscularis of the urinary bladder. The suture may be inserted at a location laterally removed from one side of the incision, pushed through both faces of the incision, and further inserted through the muscularis on the other side of the incision. Other forms of suturing patterns as described herein or as known in the art may be used.

g. Anastomosis of the Liver Bile Duct to a Bowel Structure

In one aspect, the present invention provide a method for forming an anastomosis of a liver bile duct to an intestine, comprising joining an end of the bile duct to an incision in the intestine using a suture that comprises a fibrosing agent.

Procedures described here may be used during the surgical preparation of an anastomosis of a bile duct to an intestine. The intestinal structure may be stabilized, as necessary, during the surgical procedure, as described above. In an exemplary embodiment, one end of the bile duct is connected to the liver, while the other end is free, after having been severed. An opening is made in the wall of the bowel structure to receive the annular free end of the bile duct. In one embodiment, an insertion device is used, with steps comprising placing the free end of the bile duct in contact with the opening in the bowel structure, and forming a junction at the annular contact area between the bile duct tissue and the bowel structure tissue. The first pointed end of the fibrosis-inducing suture and the leading end of the insertion device are inserted into the tissue on one side of the junction. The first end of the suture and the leading end of the insertion device are pushed through the tissue on one side of the junction, through the junction, and penetrate the tissue on the other side of the junction. The insertion device is gripped and pulled at the trailing end to remove the insertion device, leaving the suture in place in both the bile duct tissue and the bowel structure tissue. The previous steps are repeated as necessary to provide an anastomotic seal at the junction. In certain embodiments, the liver bile duct-to-bowel structure anastomosis is performed with needles.

In other embodiments, longer fibrosis-inducing sutures may be used in a wavelike or zigzag pattern, being threaded back and forth between the bile duct tissue and the intestinal wall tissue, continuing around the site of the anastomosis. It is further to be understood that other suturing patterns as described herein and as known in the art could be used to complete the anastomotic seal.

h. Tying Off an Appendiceal Stump

In one aspect, the present invention provides a method for tying off an appendiceal stump following an appendectomy. Such a method comprises: placing a suture that comprises a fibrosing agent around a base of an appendix prior to excising the appendix, excising the appendix, and drawing the sutre tight.

The appendix extends from the cecum of the large intestine and has a base with a circumference at the juncture of the appendix and the cecum. An appendectomy, i.e., surgical removal of the appendix, is usually performed in response to acute appendicitis, i.e., acute inflammation of the appendix, usually due to bacterial infection. An appendectomy leaves a portion of tissue, which may be termed an appendiceal stump. Prior to incising the appendix, a fiborsis-inducing suture (e.g., a self-retaining suture that comprises a fibrosing agent) may be placed so that it will be ready to tie off the appendiceal stump. The suture is placed around the base of the appendix, using either a curved insertion device or with curved needles. The first pointed end of the suture is inserted into the tissue of the cecum proximate to the appendix base. The first end of the suture is pushed around the circumference of the base in one direction for at least half of the circumference of the base until extending through an exit point in the tissue. The second pointed end of the suture is then inserted into tissue of the cecum proximate to the entry point of the first end, and the second end of the suture is pushed along the circumference of the base in the other direction for at least half of the circumference of the base until extending through an exit point in the tissue. The appendix is excised, leaving the appendiceal stump. The ends of the fibrosis-inducing suture are gripped and pulled, causing the suture to tighten around the appendiceal stump, and may invert the stump into the cecum.

i. Reparing a Zenker's Diverticulum

In one aspect, the present invention provides a method for reparing a Zenker's Diverticulum. Such a method comprises: placing a suture that comprises a fibrosing agent into an endoscopic insertion device, orally inserting the device through two sides of an orifice formed by the diverticulum, withdrawing the device and leaving the suture in place, using the suture to draw the two sides of the orifice together, and excising the diverticulum.

A Zenker's Diverticulum is a sac that protrudes from the esophagus below the pharynx. It is a herniation of the mucosal tissue between the fibers of the pharyngeal constrictor and cricopharyngeal muscles. The Diverticulum forms an orifice toward lumen of the esophagus.

The Zenker's Diverticulum may first be manually inverted into the esophagus, or left outside the esophagus. A fibrosis-inducing suture (e.g., a self-retaining suture that comprises a fibrosing agent) may be placed into an endosocpic insertion device, and the endoscopic insertion device is inserted orally to insert the self-retaining suture into the cricopharyngeal muscle above the orifice.

The first pointed end of the fibrosis-inducing suture and the leading end of the endoscopic insertion device are inserted through an entry point in the esophageal muscle between the parynx and the orifice, and spaced from the orifice. The first end of the suture and leading end of the insertion device are pushed through the muscle until the first end of the suture and the leading end of the insertion device extend out of the muscle at the orifice of the sac. Then the first pointed end of the suture and the leading end of the endoscopic insertion device are inserted through an opposing side of the orifice, and are pushed through the muscle until the second axial location is proximate to a central point of the orifice. The insertion device is gripped and pulled at the trailing end to remove the insertion device, leaving the suture in place. Optionally, the above steps may be repeated with additional sutures. The muscle on the two sides of the orifice is advanced together as necessary to close the orifice. The Diverticulum is then endoscopically cut and removed.

Optionally, the above steps may be repeated with additional sutures. Alternatively, a fibrosis-inducing suture may be inserted into and pushed through the tissue in a curved path around the circumference of the orifice, exiting near the point of insertion. The two ends of the suture may then be drawn taut to close the orifice, forming an alpha-type pattern.

j. Reparing Defects on the Interior Surface of a Viscus

In one aspect, the present invention provides a method for repairing a lesion on the interior surface of a viscus structure. Such a method comprises joining the two sides of the lesion together using suture that comprises a fibrosing agent.

Viscus structures include organs of the digestive, urogenital, respiratory, endocrine, and vascular systems, as well as the spleen and the heart. Ulcerative tissue lesions on the interior surface of a viscus may be joined and held closed by use of self-retaining sutures described herein. The lesion may be located with an endoscope and a fibrosis-inducing suture inserted using an endoscopic insertion device. When used in repairing lesions of the digestive system, for example, the devices may be inserted anally or oropharygeally. In one embodiment, the method may comprise the step of inserting the first pointed end of the suture and the leading end of the endoscopic insertion device through an entry point in the tissue spaced from, and on one side of, the lesion. Then, the first end of the suture and leading end of the insertion device are pushed through the tissue until the first end of the suture and the leading end of the insertion device extend out of the tissue at the lesion. The first pointed end of the suture and the leading end of the endoscopic insertion device are then inserted through an opposing side of the lesion, and are pushed until the second axial location is proximate to a central point of the lesion. The insertion device is gripped and pulled at the trailing end to remove the insertion device, leaving the fibrosis-inducing suture in place. Optionally the above steps may be repeated. The tissue on the two sides of the lesion is advanced together to close the lesion.

k. Joining a Foreign Element and Bodily Tissue

In one aspect, the present invention provides a method for attaching a foreign element to a surrounding body tissue, comprising joining a periphery of the foreign element to the tissue using a suture that comprises a fibrosing agent.

The foreign element (such as a prosthesis, medical device or surgical implant) has a periphery and the bodily tissue has a fibrous tissue ring. A face of the fibrous ring defines an opening and abuts a face of the periphery (of the foreign element), holding closed a junction between the element and the tissue. In one embodiment, the first pointed end of a fibrosis-inducing suture is inserted into the periphery of the foreign element at a point radially spaced from the face of the tissue ring. The first end of the suture is pushed through the periphery until the first end of the suture extends out of the periphery at an exit point and penetrates the tissue of a face of the tissue ring until the first end of the suture extends out of the tissue at an exit point radially spaced from the junction and spaced along the tissue ring circumference in a first direction from the point of insertion of the first end of the suture on the periphery. The first end of the suture is gripped and pulled, drawing the first portion of the suture through the periphery and the tissue while bringing the periphery and the tissue together to a closed position along the first portion of the suture. This continues until the second axial location is at the point of insertion of the first end of the suture in the periphery and a length of the first portion of the suture is left in the periphery and the tissue between the point of insertion and the exit point. Then the first end of the suture is inserted into the tissue at the exit point of the first end. The first end is pushed through the tissue until the first end extends out of the tissue at an exit point in the face of the tissue ring and penetrates the periphery until the first end extends out of the periphery at an exit point radially spaced from the junction and spaced along the circumference of the tissue ring in the first direction from the immediately preceding point of insertion of the first end of the suture in the periphery. The first end of the suture is gripped and pulled out of the periphery, drawing the first portion of the suture through the periphery and tissue while bringing the periphery and the tissue together to a closed position along the first portion of the suture, and leaving a length of the first portion of the suture in the periphery between the point of insertion and the exit point. The above steps are repeated, with each repetition advancing the suture around the circumference of the junction in a first direction. Further, the above steps are repeated similarly for a second end and second portion of the suture, in a second direction. In addition, a similar method may be carried out with the suture first being inserted in the tissue rather than in the periphery of the foreign element. The sutures may extend completely around the circumference of the junction, and may overlap one quarter or more of the circumference. Optionally to the method described, the suture may be inserted and advanced by entering or exiting the tissue or the periphery of the element only at the face of the junction between the two, but not on the surface of the tissue or foreign element.

In certain embodiments of this method, the foreign element is a replacement heart valve, with an annular cuff of the valve forming its periphery. The cuff is joined to the fibrous muscle tissue of the heart surrounding the location of the replacement valve. The replacement heart valve may be bioprosthetic or mechanical. In certain other embodiments, the foreign element may be synthetic vascular grafts and bioprosthetics for correcting cadiac septal defects.

l. Mounting a Device to Body Tissues

In one aspect, the present invention provides a method for mounting a device to a body tissue. Such a method may comprise securing a device to a body tissue using a suture that comprises a fibrosing agent.

The device to be mounted to a body tissue may include, for example, at least one eyelet for securing the device to the body and through which a fibrosis-inducing suture may pass. In one embodiment, the method starts with the step of placing the device in a desired anatomical position. Then a suture is threaded through the eyelet. The first pointed end of the suture is inserted into tissue and is pushed through the tissue until extending out an exit point. The first end of the suture is gripped and pulled out of the tissue while drawing the first portion of the suture through the tissue, leaving a portion of the suture between the first and second axial locations out of the tissue and leaving a length of the first portion of the suture in the tissue between the point of insertion and exit point of the first end. These steps are repeated for the second end and portion of the suture in a second direction, resulting in the first and second portions of the suture extending in the tissue in generally opposing directions and causing the suture to resist displacement of the device. Examples of devices that may be mounted include catheters, electrodes of cardiac pacemakers and defibrillators, neurostimulation electrodes and tumor monitors. The device may be mounted internally, for example, to an organ, or externally to the epidermis.

m. Endoluminal Procedures

In one aspect, the present invention provides a method for fastening an endoluminal organ or a portion thereof, comprising typing an endoluminal organ or a portion thereof with a self-retaining suture that comprises a fibrosing agent.

Self-retaining sutures may be used in a variety of applications to increase the integrity of a damaged or perforated body tube or to secure an endoluminal graft to the wall of a body tube (e.g., a blood vessel). Self-retaining sutures may also be used in a variety of endoluminal surgeries, such as, but not limited to, transmural polypectomies, resections of submucosal lesions, bowel resections, resection of processes such as ulcers, controlling of bleeding, closing perforations, prophylactic or therapeutic appendectomies, resection of bleeding diverticuli or Meckel's diverticulum, anchoring tubes or grafts (e.g., anastomosis of a vascular artery to a graft), securing time-released medications, performing gastroplasty, fallopian tube ligation, solid organ biopsies, bowel structuring, or partial lung resection.

Self-retaining sutures may be used in endoluminal applications by insertion endoscopically through a natural entrance to the interior of the body tube, such as entrance to the gastrointestinal tract either anally or oropharyngeally. Sutures may alternatively be introduced using an insertion device, through the wall of the body tube, such as insertion into a blood vessel through the walls of the abdomen and the vessel.

Endoluminal procedures using self-retaining sutures for the repair of a Zenker's Diverticulum in the esophagus or the repair of an endoluminal lesion in a viscus structure are described above.

n. Skin Graft

In one aspect, the present application provides a method for performing a skin graft. Such a method comprises holding portions of the graft to underlying tissue using a suture, a suture anchor or a suture connector that comprises a fibrosing agent. For example, a series of suture connectors can be inserted into tissue, along the edges and in the field of a skin graft to hold the graft in place, with the connectors residing beneath the skin until healing has occurring, thus reducing surface scarring.

In certain embodiments, the sutures, suture anchors or suture connectors are absorbable. In certain other embodiments, they are non-absorbable.

o. Nerve Repairs

In one aspect, the present invention provides method for repairing nerves. Such a method comprises joining the nerves together with a suture that comprises a fibrosing agent.

Nerve lacerations often require primary fixation, with sutures used to approximate the nerve and align the nerve fascicles. Suturing nerves presents technical challenges given the very small size of the structures and sutures and obstructive nature of the knots. In one embodiment, self-retaining sutures that comprise a fibrosing agent can be used to simplify the nerve repair procedure by eliminating the needs for knots. The nerve and its fascicles are visually aligned and an anchoring self-retaining suture is run from the proximal end of the nerve, through the perineurium connective tissue into the distal nerve perineurium connective tissue with the bidirectional retainers anchoring the two structures. The distal end of the suture is brought back out of the nerve and held while the nerve is approximated to eliminate a gap between the nerves. The exposed self-retaining suture is then cut.

p. Vaginal Volume Reduction

In one aspect, the present invention provides a method for reducing vaginal volume. Such a method comprises tightening vaginal wall using a suture that comprises a fibrosing agent.

A number of woman post partum complain of internal vaginal laxity and often request a procedure consisting of elliptical tissue removal from the vaginal vault to tighten the vagina internally. A suture that comprises a fibrosing agent offers a minimally invasive solution. In one embodiment, a self-retaining suture that comprises a fibrosing agent is run from the distal vagina towards the proximal vagina intramuscularly in a longitudinal fashion with the suture exiting proximally. The vagina wall is then tightened by manually adjusting the vaginal wall along the barbs, which would also increase the prominence of the internal vaginal rugae. The excess suture is then cut. A number of the longitudinally running sutures may be place equally dispersed radially within the vaginal, 4 to 8 being optimal.

q. Incontinence

In one aspect, the present invention provides a method for treating incontinence. Such a method comprises tightening the external spinter using a suture that comprises a fibrosing agent.

Both urinary and bowel incontinence have a component of external sphincter dysfunction. In one embodiment, self-retaining sutures that comprise a fibrosing agent may be run through the external sphincter to tighten the structure and help control incontinence. The self-retaining suture is anchored with a knot at the insertion point, and then the suture is run around the external sphincter exiting in area of the original introduction and exits the muscle. The sphincter is then manually tightened by manually or with the aid of an instrument ratcheting the tissue along the self-retaining suture. The exiting suture is then fixed with a knot on the external sphincter.

r. Diapharmatic Hernia

In one aspect, the present invention provides a method for treating diapharmatic hernia. Such a method comprises tightening the diaphragm using a suture that comprises a fibrosing agent.

A sliding hiatus hernia is a common cause of gastrointestinal difficulties. Often the diaphragm does not provide a tight enough structure around the proximal stomach, which then permits the stomach to “slide” proximally and cause symptoms. The reparative procedure is an invasive and difficult solution. A suture (e.g., a self-retaining suture) that comprises a fibrosing agent would offer a minimally invasive solution to tightening the diaphragmatic ring. In one embodiment, laproscopically a self-retaining suture is introduced from the inferior aspect of the diaphragm into the diaphragmatic hernia site. The suture is fixated to the diaphragm with a knot at point of entry then run in a circle around the hernia and tensioned until the desired aperture size is achieved. The distal end of the suture is then fixated to the diaphragm with a knot.

s. Senile Vocal Cord Atrophy

In one aspect, the present invention provides a method for treating senile vocal cord atrophy. Such a method comprises tensioning a vocal cord in need thereof using a suture that comprises a fibrosing agent.

With increasing age, the vocal cords become lax and no longer tightly approximate leaving the individual with an elderly sounding voice. In one embodiment, a fibrosing suture (e.g., a biodegradable fibrosing suture) is run through the vocal cord and then tensioned in order to take up the slack resulting in a more youthful voice.

t. Lip Augmentation

In one aspect, the present invention provides a method for augmenting the lip. Such a method comprises implanting one or more sutures inside the lip, wherein the one or more sutures comprise a fibrosing agent.

Lip augmentation is commonly performed with collagen or hyaluronic injections, however there are only temporary by nature. Sutures (e.g., self-retaining sutures) that comprise a fibrosing agent may offer a permanent solution. In one embodiment, four self-retaining sutures would be placed (2 upper lip and 2 lower lip) effectively rolling out the lips to make them fuller. Anchoring suture is placed in the mouth at the angle of where the inner lips meet the inner mucosa attaches to the mandible inferiorly and the maxilla superiorly, and the self-retaining suture is then run at an angle to capture the lip at the transition area externally. The lip tissue is then tensioned along the self-retaining suture, effectively rolling out and making the deeper colored aspect of the lip more prominent, creating a fuller looking lip.

u. Abdominal Augmentation.

In one aspect, the present invention provides a method for abdominal augmentation. Men desire the physique of a well-trained individual with well-defined abdominal muscles. In one embodiment, dissolvable fibrosis inducing self-retaining sutures can be placed across the fat tissue superficial to the rectus abdominins then tensioned in an orientation to enhance or create a tissue fold that is aligned with the rectus abdominins tendinous insertions, giving the appearance of a well defined abdomin.

v. Deceased Cosmetics

In one aspect, the present invention provides a method for deceased cosmetics. After the death of an individual, great care and attention is directed towards making the deceased look presentable at their funeral. In one embodiment, self-retaining sutures can be used to alter the facial expression of the deceased to create the desired look for a funeral.

### EXAMPLES

### Example 1

### Preparation of Silk Powder from Degummed Silk Using a Cryomill

Fibers of degummed silk were cut into pieces approximately 1-2 cm in length. The material was then milled to a powder using a cryom ill (Spex Certiprep Freezer/Mill—Model 6850). A portion of the milled powder was then sieved through a series of different sized metal sieves to obtain silk powder of different size ranges.

### Example 2

### Preparation of Silk Powder

Several pieces of silk braid (Eth icon, 4-0, 638) are cut into lengths of approx 0.4 cm. These cut pieces are placed in a 100 ml round bottom flask that contains 50 ml 2M NaOH. The sample is stirred using a magnetic stirrer at room temperature for 24 h. The sample is neutralized using concentrated HCl. The neutralized contents are then dialyzed against deionized water using cellulose-based dialysis tubing (WMCO approx 3000; Spectrum). The sample is dialyzed for 48 hours with 5 water changes. The dialyzed sample is then poured into a 100 ml round bottom flask. The sample is frozen and freeze-dried to yield a fluffy powdered material.

### Example 3

### Preparation of Silk Powder from Silk Braid Using a Cryomill

Several pieces of silk braid (Eth icon, 4-0, 638) are cut into lengths of approx 0.4 cm. These were then milled in a cryomill (Freezermill) for 20 minutes. The powder was then passed through a 53 um stainless steel sieve. The silk powder was collected and stored in a glass vial.

### Example 4

### Silk Coating and Barbing of a Degradable Suture

250 mg Cryomilled silk powder (<25 um in diameter) from degummed silk [Example 1] is added to glass jar that contains a 5 g low molecular weight glycolide/ε-caprolactone copolymer in methylene chloride (50 mL). The jar is then placed on a roller mill and allowed to mix for 24 hours. The dispersion in then placed in a coating bath. A monofilament polydioxanone suture (size 3-0) is then pulled through the coating bath that contains the silk/polymer coating solution at a speed of 8 feet per minute with a residence time of about 5 seconds in the coating bath. The suture is dried to a tack free state after passing through a drying chamber after which it is wound onto a spool. The product is further dried under vacuum for 24 hours. The suture is then barbed according to U.S. Pat. No. 6,848,152.

### Example 5

### Silk Coating of a Self-Retaining Degradable Suture

250 mg Cryomilled silk powder (<25 μm in diameter) is added to a glass jar containing 5 g of low molecular weight glycolide/c-caprolactone copolymer (10:90 w/w) in methylene chloride (50 mL). The jar is then placed on a roller mill and allowed to mix for 24 hours. The dispersion is then placed in a coating bath. The coating bath is stirred to ensure the dispersion does not settle. A monofilament polydioxanone suture (size 3-0) is then pulled through the coating bath that contains the silk/polymer coating solution. The suture is air dried to a tack-free state, after which it is further dried under vacuum for 24 hours. The coated suture is then transformed into a self-retaining suture according to U.S. Pat. No. 6,848,152.

### Example 6

### Coating of a Self-Retaining Suture with Fibronectin

A 1% (w/w) fibronectin (Calbiochem, San Diego, Calif.) solution in sterile water is prepared. A monofilament polydioxanone suture (size 3-0), self-retaining according to U.S. Pat. No. 6,848,152, is incubated in the fibronectin solution for 1 minute. The suture is removed and then air-dried. The suture is dried under vacuum for 24 hours.

### Example 7

### Coating of a Self-Retaining Suture with Poly-L-Lysine

A 1% (w/w) poly-L-lysine (Sigma Aldrich, St. Louis, Mo.) solution in sterile water is prepared. A monofilament polydioxanone suture (size 3-0) is coated using the procedure described in Example 6.

### Example 8

### Coating of a Self-Retaining Suture with N-Carboxybutyl Chitosan

A 1% (w/w) n-carboxybutyl chitosan (Carbomer, Westborough, Mass.) solution in sterile water is prepared. A monofilament polydioxanone suture (size 3-0) is coated using the procedure described in Example 6.

### Example 9

### Coating of a Self-Retaining Suture with Bromocriptine in Polyester

A 4.5% w/w solution of low molecular weight glycolide/ε-caprolactone copolymer (10:90 w/w) in methylene chloride is prepared. Bromocriptine mesylate (Sigma Aldrich) is dissolved/suspended in this solution at 5 mg/ml. A monofilament polydioxanone suture (size 3-0) is coated using the procedure described in Example 5.

### Example 10

### Preparation of Inflammatory Microcrystals (Monosodium Urate Monohydrate and Calcium Pyrophosphate Dihydrate)

Monosodium urate monohydrate (MSUM) microcrystals were grown. A solution of uric acid and sodium hydroxide at 55° C. and pH 8.9 was left to stand overnight at room temperature. The crystals were rinsed several times with cold (4° C.) distilled water and dried at 60° C. for 12 hours in a circulating hot-air oven (Fisher, Isotemp).

Triclinic calcium pyrophosphate dihydrate (CPPD) crystals were prepared as follows. A 250 ml beaker containing 103 ml distilled water was heated in a water bath to 60° C., and stirred constantly with a TEFLON-coated stir bar. The stirring was slowed and 0.71 ml of concentrated hydrochloric acid and 0.32 ml of glacial acetic acid were added, followed by 0.6 g of calcium acetate. A 150 ml beaker containing 20 ml distilled water was heated to 60° C. in the water bath, and 0.6 g calcium acetate added. The rate of stir was increased in the 250 ml beaker, and 2 g of calcium acid pyrophosphate added rapidly. When the CaH₂P₂O₇ had nearly all dissolved, the rate of stirring was reduced for 5 minutes. Then over a period of 15 seconds, the contents of the small beaker were poured into the large beaker with vigorous stirring. In the preparation of subsequent batches, a minute amount of triclinic CPPD crystals was added to the large beaker as seed material. Stirring was discontinued, leaving a white gel. This was allowed to remain undisturbed in the cooling water bath. The pH of the supernatant was always less than 3.0. The gel collapsed as CPPD crystals formed in 24 hours. The crystals were washed in distilled water 3 times, washed in ethanol, then washed in acetone, and air-dried.

### Example 11

### Coating of a Self-Retaining Suture with Inflammatory Microcrystals (Monosodium Urate Monohydrate or Calcium Pyrophosphate Dihydrate) from Polymer Solution

A 4.5% w/w solution of low molecular weight glycolide/ε-caprolactone copolymer (10:90 w/w) is prepared in dichloromethane. Inflammatory microcrystals (MSUM or CPPD) are ground in a pestle and mortar to a particle size of 10 to 50 micrometers and suspended in the solution at 5 mg/ml. A monofilament polydioxanone suture (size 3-0) is coated using the procedure described in Example 5.

### Example 12

### Coating of a Self-Retaining Suture with Inflammatory Microcrystals (Monosodium Urate Monohydrate or Calcium Pyrophosphate Dihydrate)

A 4.5% w/w solution of poly (lactide co-glycolide) (85:15) (IV 0.61) (Birmingham Polymers, Birmingham, Ala.) blended with methoxypolyethylene glycol 350 (MePEG 350) (Union Carbide, Danbury, Conn.) in a ratio of 80:20 w/w (PLGA:MePEG) is prepared in dichloromethane. Inflammatory microcrystals are suspended in the solution at 5 mg/ml. A monofilament polydioxanone suture (size 3-0) is coated using the procedure described in Example 5.

### Example 13

### Transforming Growth Factor-β (TGF-β)—Coated Self-Retaining Suture

A 1% solution of hyaluronic acid (HA) (sodium salt, Sigma Aldrich) in water, containing 30% glycerol (w/w to HA) (Fisher Scientific) and 8 mM 1-ethyl-3-(−3 dimethylaminopropyl) carbodiimide (EDAC) is prepared by dissolution overnight. TGF-β (Calbiochem, San Diego, Calif.) is dissolved at 0.01 mg/ml in this solution. A monofilament polydioxanone suture (size 3-0) is self-retaining according to U.S. Pat. No. 6,848,152 is incubated in the fibronectin solution for 1 minute. The suture is removed and then air-dried. The suture is dried under vacuum for 24 hours.

### Example 14

### Coating of a Self-Retaining Suture with Fibroblast Growth Factor (FGF) in Crosslinked Chitosan

A 1% solution of chitosan (Medical grade, Carbomer, Westborough, Mass.) in dilute acetic acid (pH 5), containing 30% glycerol (w/w to chitosan) (Fisher Scientific) and 0.5% glutaraldehyde (Sigma, St. Louis, Mo.) is prepared by dissolution overnight. FGF (Calbiochem, San Diego, Calif.) is dissolved at 0.01 mg/ml in this solution. A monofilament polydioxanone suture (size 3-0) is coated using the procedure described in Example 6.

### Example 15

### Silk Coating and Barbing of a Non-Degradable Suture

250 mg Cryomilled silk powder (<25 um in diameter) is added to glass jar that contains a 5 g low molecular weight glycolide/ε-caprolactone copolymer in methylene chloride (50 mL). The jar is then placed on a roller mill and allowed to mix for 24 hours. The dispersion in then placed in a coating bath. A monofilament polypropylene suture (size 3-0) is then pulled through the coating bath that contains the silk/polymer coating solution at a speed of 8 feet per minute with a residence time of about 5 seconds in the coating bath. The suture is dried to a tack free state after passing through a drying chamber after which it is wound onto a spool. The product is further dried under vacuum for 24 hours. The suture is then barbed according to U.S. Pat. No. 6,848,152.

### Example 16

### Method for Performing a Facelift

Using a silk-coated self-retaining non-degradable suture (Example 15), one end is inserted using a needle at the temporal hairline forward of the top connection point of the ear. The tip of the suture is advanced through the subepidermal tissue beneath the scalp, extending distally toward a location above the ear. The other end of the suture is inserted at the same location and the tip is extended toward the nasolabial fold, engaging the subepidermal tissue, engaging the subepidermal tissue and/or the superficial muscular aponeurotic system, and exiting distally. As tension is maintained on the free ends of the suture, the engaged tissues on the lower end are manually drawn toward the insertion point to achieve the lifting effect. The procedure is repeated on the opposite side of the face.

### Example 17

### Assessing Tissue Holding Performance of Self-Retaining Sutures

The tissue holding capacity of silk coated self-retaining degradable sutures prepared according to Example 5, silk coated self-retaining non-degradable sutures prepared according to Example 6 are tested, self-retaining degradable sutures without silk coating prepared according to Example 5, and self-retaining non-degradable sutures without silk coating prepared according to Example 6 are tested using an in vitro mechanical tester to determine the force required to induce a 2 mm gap orthogonal to a wound which had been closed

A 1 cm long incision are created in a skin stimulant (Darra chamois) and closed with the above-described sutures. Ten sutured tissue specimens of each suture type are tested on a tensile tester (Sintech). Each test continues until a 2 mm gap occurred at any point along the incision line.

### Example 18

### In Vivo Suture Strength and Histopathology Evaluations

Self-retaining Sutures: Self-retaining degradable sutures without silk coating and those with silk coating are prepared according to Example 5, and self-retaining non-degradable sutures and those with silk coating are prepared according to Example 6.

In Vivo Suture Tensile Strength: The above-described sutures are implanted into the subcutis layer of eight New Zeland White rabbits, via a large bore catheter. A total of at least four sutures, placed parallel to the dorsal midline, are implanted in each rabbit. At 2, 4, 5, and 8 weeks postoperatively, two animals are sacrificed respectively and the corresponding sutures retrieve for tensile strength testing. They are compared to the suture tensile strength before implantation (week 0).

In Vivo Retainer Holding Strength: At pre-determined, randomized locations in the dorsal aspect of four dogs, the sutures are placed into the dermal layer of the skin with a straight needle, burying at least 3 cm of the self-retaining section. For each dog, 6 sutures are implanted at the end of each week up to 3 weeks, resulting in implantation times of equivalent durations. Immediately after implantation on the end of the 3 weeks, all animals are sacrificed. The segment of the skin and deep subcutaneous tissue containing the suture are excised, clamped and fastened onto a Digital Force Gauge (Chatillon DFIS2) or a NorMark Weigh-in Electronic Digital Scale (Model 15). The free suture end is secured by knots to a stainless steel suture that is attached to a Syringe Pump (KD Scientific Model 220), set to pull at 2.9 cm/min. The retainer holding strength is determined as the peak force recorded by pulling the suture from the tissue.

Histopathology Evaluations:

1) Canine Dermis. Selected suture sites (2-4 for each time period) from the retainer holding strength experiment above are harvested for histopathology. Histopathological evaluation is modeled after a scoring protocol devised by Scott et al. Small Animal Dermatology, WB Sauders Company, Philadelphia, 1995, 5^{th} Ed, p55-174. Specifically, each slide (stained with hematoxyl in and eosin) is examined and a score of 0 (no significant findings), 0.5 (rare or very slight), 1 (slight), 2 (moderate), or 3 (marked) is assigned to each of the following parameters—polymorphonuclear leukocytes, lymphocytes, eosinophils, plasma cells, mast cells, macrophages, giant cells, granulation tissue, fibrosis, hemorrhage, necrosis, degeneration, foreign debris, and relative size of involved area.

2) Rabbit Muscle. The above-described sutures are implanted into the paravertebral muscle of six New Zealand White rabbits, on opposite sides of the spine. Three animals are maintained for 8 weeks and the other three 16 weeks. At sacrifice, 3 suture and 3 control sites of each animal are processed for histopathological evaluation. Scoring is similar to above.

### Example 19

### Wound Closure Efficacy Study

Three Mongrel dogs are used. Multiple incisions are made at the thorax, thigh, flank, ventral midline, and paramedian on each animal. The incisions are closed with self-retaining sutures with or without silk coating prepared according to Example 5 or Example 6, respectively. All animals are monitored daily and maintained for 14 days. In various tissues, incision sizes and locations on the dogs and all incisions apposed with the self-retaining sutures are monitored throughout the observation period.

### Example 20

### In Vivo Wound Security

Two different models—rat abdomen and porcine skin are employed in this study.

Rat Abdomen. A total of 40 rats are used. A 60 mm wound is created through the abdominal wall after it is exposed following a midline skin incision. The wound is closed by silk coated self-retaining degradable sutures prepared according to Example 5 (10 rats), silk coated self-retaining non-degradable sutures prepared according to Example 6 (10 rats), degradable sutures without silk coating prepared according to Example 5 (10 rats), and non-degradable sutures without silk coating prepared according to Example 6 (10 rats), respectively. Analysis at 0, 2, and 4 weeks included wound strength (10 rats at each time point) and histology (5 rats each at 2 and 4 weeks).

Porcine Dermis. A total of 3 pigs are employed with one for each of the three analysis periods—0, 2, and 6 weeks. Twelve sets of four full-thickness, 4-cm dosolateral skin incisions are made on each pig. Each set of four incisions is closed with a silk coated self-retaining degradable sutures prepared according to Example 5, a silk coated self-retaining non-degradable sutures prepared according to Example 6, a degradable sutures without silk coating, and a non-degradable sutures without silk coating, respectively. One pig is sacrificed at each analysis time point for wound strength, histology, and cosmesis.

Wound Strength Measurement. Each excised tissue specimen is mounted on an instron (Model 1123) tensiometer and separated at a rate of 20 mm/min for peak force determination.

Histological Evaluation. Modeled after a protocol by Scott et al. (Small Animal Dermatology, WB Saunders Company, Philadelphia, 1995, 5^{th} Ed, p55-174), the tissue response of each suture is rated from none (0) to marked (3), based on the following parameters: degree of necrosis, coagulative necrosis, floccular degeneration, inflammation, neutrophils, lymphocytes, plasma cells, macrophages, fibroendothelial proliferation, fibrosis, giant cells, and fatty infiltration.

Cosmetic Evaluation. Each wound is evaluated by a blinded plastic surgeon using the visual analog scale (VAS) [0 (worst) to 100 (best)], and the modified Hollader scale [the sum of individual scores of 0 (absent) or 1 (present) for stepoff of borders, contour irregularities, margin separation, edge inversion, excessive distortion, and unacceptable overall appearance].

### Example 21

### Rat Model to Assess Fibrosis-Inducing Agents in Rats

The rat caecal sidewall model is used to as to assess the fibrosis-inducing capacity of formulations in vivo. Sprague Dawley rats are anesthetized with halothane. Using aseptic precautions, the abdomen is opened via a midline incision. The caecum is exposed and lifted out of the abdominal cavity. The caecum is then positioned over an area of the sidewall and attached using two standard sutures. The fibrosis-inducing formulation, e.g., a silk-coated self-retaining suture or a silk gel formulation, is positioned between the two tissues when the self-retaining suture is used or is applied over both sides of the caecum and over the peritoneal sidewall when the gel is administered. Two additional standard sutures are placed to attach the caecum to the sidewall, and the abdominal incision is closed in two layers. After 7 days, animals are evaluated post mortem with respect to the extent and severity of tissue reaction, which are scored both quantitatively and qualitatively. Exemplary materials that may be tested in this model include silk and talc.

### Example 22

### Rabbit Model to Assess Fibrosis-Inducing Agents

The rabbit uterine horn model is used to assess the fibrotic capacity of formulations in vivo. Mature New Zealand White (NZW) female rabbits are placed under general anesthetic. Using aseptic precautions, the abdomen is opened in two layers at the midline to expose the uterus. Both uterine horns are lifted out of the abdominal cavity and assessed for size on the French Scale of catheters. Horns between #8 and #14 on the French Scale (2.5-4.5 mm diameter) are deemed suitable for this model. The individual horns are then opposed to the peritoneal wall and secured by two standard sutures placed 2 mm beyond the edges of the abraded area. The scar-inducing formulation is applied to the horns and the abdomen is closed in three layers. After 14 days, animals are evaluated post mortem with the extent and severity of scarring being scored both quantitatively and qualitatively. Exemplary compounds that may be tested in this model include for example, silk and talc.

### Example 23

### In Vivo Evaluation of Silk-Coated Perivascular PU Films to Assess Scarring

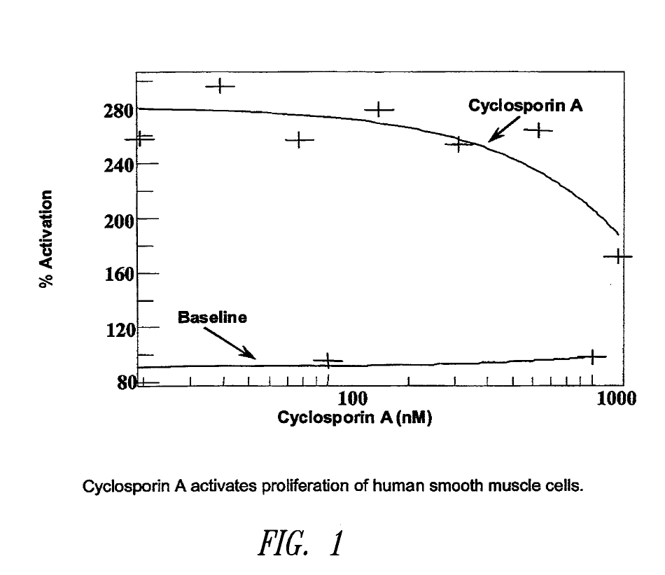

A rat carotid artery model was described for determining whether a substance stimulates fibrosis. Wistar rats weighing 300 g to 400 g were anesthetized with halothane. The skin over the neck region was shaved and the skin is sterilized. A vertical incision was made over the trachea, and the left carotid artery was exposed. A polyurethane film covered with silk strands or a control uncoated PU film was wrapped around a distal segment of the common carotid artery. The wound was closed and the animal is allowed to recover. After 28 days, the rats were sacrificed with carbon dioxide and then pressure-perfused at 100 mmHg with 10% buffered formaldehyde. Both carotid arteries were harvested and processed for histology. Serial cross-sections were cut every 2 mm in the treated left carotid artery and at corresponding levels in the untreated right carotid artery. Sections were stained with H&E and Movat's stains to evaluate tissue growth around the carotid artery. Area of perivascular granulation tissue was quantified by computer-assisted morphometric analysis. Area of the granulation tissue was significantly higher in the silk-coated group than in the control uncoated group (see FIG. 1).

### Example 24

### In Vivo Evaluation of Perivascular PU Films Coated with Different Silk Suture Material to Assess Scarring

A rat carotid artery model is described for determining whether a substance stimulates fibrosis. Wistar rats weighing 300 g to 400 g were anesthetized with halothane. The skin over the neck region was shaved and the skin was sterilized. A vertical incision was made over the trachea and the left carotid artery was exposed. A polyurethane film covered with silk sutures from one of three different manufacturers (3-0 Silk—Black Braided (Davis & Geck); 3-0 SOFSILK (U.S. Surgical/Davis & Geck); and 3-0 Silk—Black Braided (LIGAPAK) (Ethicon, Inc.)) was wrapped around a distal segment of the common carotid artery. (The polyurethane film can also be coated with other agents to induce fibrosis.) The wound was closed and the animal was allowed to recover.

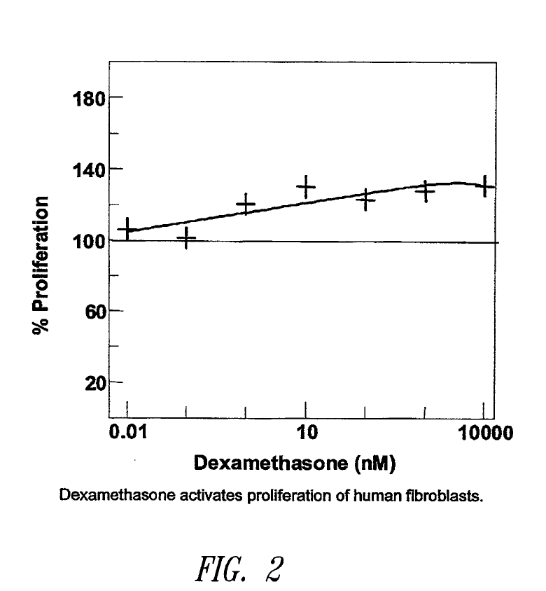

After 28 days, the rats were sacrificed with carbon dioxide and then pressure-perfused at 100 mmHg with 10% buffered formaldehyde. Both carotid arteries were harvested and processed for histology. Serial cross-sections were cut every 2 mm in the treated left carotid artery and at corresponding levels in the untreated right carotid artery. Sections were stained with H&E and Movat's stains to evaluate tissue growth around the carotid artery. Area of perivascular granulation tissue was quantified by computer-assisted morphometric analysis. As illustrated in FIG. 2, thickness of the granulation tissue was the same in the three groups, showing that tissue proliferation around silk suture is independent of manufacturing processes.

### Example 25

### In Vivo Evaluation of Perivascular Silk Powder to Assess Scarring

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. Silk powder is sprinkled on the exposed artery that is then wrapped with a PU film. Natural silk powder or purified silk powder (without contaminant proteins) is used in different groups of animals. Carotids wrapped with PU films only are used as a control group. The wound is closed and the animal is allowed to recover. After 28 days, the rats are sacrificed with carbon dioxide and then pressure-perfused at 100 mmHg with 10% buffered formaldehyde. Both carotid arteries are harvested and processed for histology. Serial cross-sections are cut every 2 mm in the treated left carotid artery and at corresponding levels in the untreated right carotid artery. Sections are stained with H&E and Movat's stains to evaluate tissue growth around the carotid artery. Areas of tunica intima, tunica media, and perivascular granulation tissue are quantified by computer-assisted morphometric analysis.

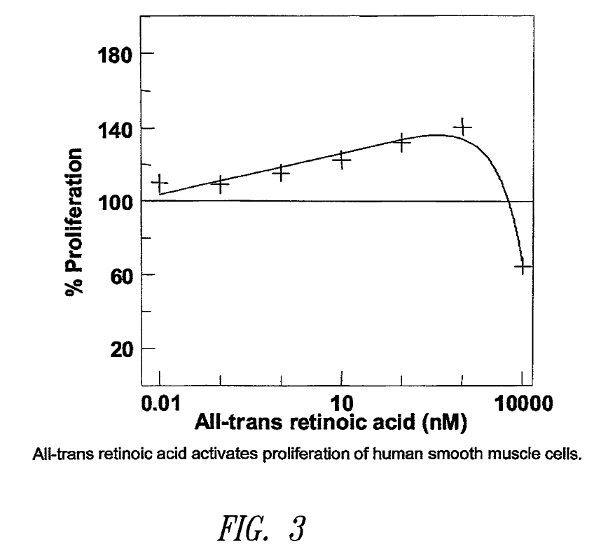

In one experiment conducted on a group of six rats, the natural silk caused a severe cellular inflammation consisting mainly of a neutrophil and lymphocyte infiltrate in a fibrin network without any extracellular matrix or blood vessels in 2/6 cases. In addition, the treated arteries were seriously damaged with hypocellular media, fragmented elastic laminae and thick intimal hyperplasia. Intimal hyperplasia contained many inflammatory cells and was occlusive in 2/6 cases. This severe immune response was likely triggered by antigenic proteins coating the silk protein in this formulation. On the other end, the regenerated silk powder triggered only a mild foreign body response surrounding the treated artery. This tissue response was characterized by inflammatory cells in extracellular matrix, giant cells, and the presence of supportive blood vessels. The treated artery was intact. These results show that removing the coating proteins from natural silk prevents the immune response and promotes benign tissue growth. Degradation of the regenerated silk powder was underway in some histology sections indicating that the tissue response can likely mature and heal over time. The area of granulation tissue is presented in FIG. 3.

### Example 26

### In Vivo Evaluation of Perivascular Talcum Powder to Assess Scarring

A rat carotid artery model is described for determining whether a substance stimulates fibrosis. Wistar rats weighing 300 g to 400 g were anesthetized with halothane. The skin over the neck region was shaved and the skin is sterilized. A vertical incision was made over the trachea and the left carotid artery was exposed. Talcum powder was sprinkled on the exposed artery that was then wrapped with a PU film. Carotids wrapped with PU films only were used as a control group. The wound was closed and the animal is allowed to recover. After 1 or 3 months, the rats were sacrificed with carbon dioxide and then pressure-perfused at 100 mmHg with 10% buffered formaldehyde. Both carotid arteries were harvested and processed for histology. Serial cross-sections were cut every 2 mm in the treated left carotid artery and at corresponding levels in the untreated right carotid artery. Sections were stained with H&E and Movat's stains to evaluate tissue growth around the carotid artery. Thickness of tunica intima, tunica media, and perivascular granulation tissue was quantified by computer-assisted morphometric analysis.

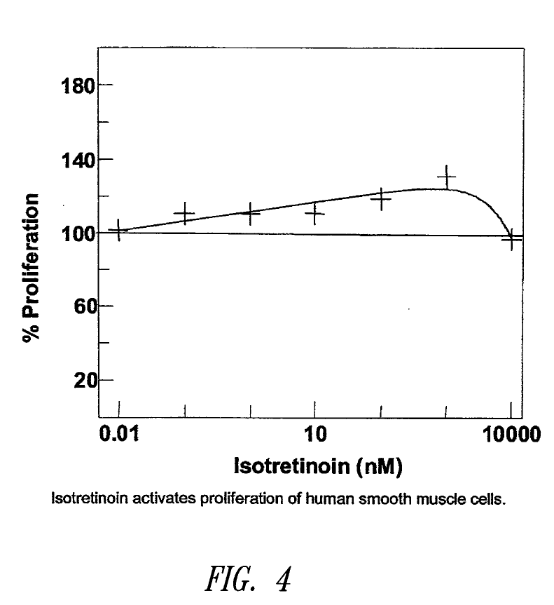

Histopathology results and morphometric analysis showed the same local response to talcum powder at 1 month and 3 months. A large tissue reaction trapped the talcum powder at the site of application around the blood vessel. This tissue was characterized by a large number of macrophages within a dense extracellular matrix with few neutrophiles, lymphocytes, and blood vessels. The treated blood vessel appeared intact and unaffected by the treatment. Overall, this result showed that talcum powder induced a mild long-lasting fibrotic reaction that was subclinical in nature and did not harm any adjacent tissue (see FIG. 4).

### Example 27

### Coating of a Suture with a Powdered Silk/PLGA Coating

A monofilament polydioxanone suture (size 3-0), either self-retaining or not self-retaining, is attached to a stainless steel rod (with the aid of a bulldog clip), which is attached to a Fisher overhead stirrer that is orientated vertically. The stirrer is set to rotate at 30 rpm. A 2% PLGA (9K, 50:50, Birmingham Polymers) solution (ethyl acetate) that contains the powdered silk is sprayed onto the rotating implant using an airbrush spray device. The concentration of the powdered silk in the PLGA solution is altered from 0.1% to 50%. After the spraying process, the suture is allowed to air dry for 30 minutes while still rotating. The suture is then removed from the rod and is further dried under vacuum for 24 h.

### Example 28

### Coating of a Suture with a Powdered Silk/Polyester Coating

A monofilament polypropylene suture (size 3-0), either self-retaining or not self-retaining, is attached to a stainless steel rod (with the aid of a bulldog clip), which is attached to a Fisher overhead stirrer that is orientated vertically. The stirrer is set to rotate at 30 rpm. A 2% low molecular weight glycolide/ε-caprolactone copolymer (10:90 w/w) in methylene chloride that contains powdered silk (prepared using a cryomill) is sprayed onto the rotating suture using a TLC spray device. The concentration of the powdered silk in the polyurethane solution is altered from 0.1% to 50%. After the spraying process, the suture is allowed to air dry for 30 minutes while still rotating. The suture is then removed from the pipette tip and is further dried under vacuum for 24 h.

### Example 29

### Top Coating of a Suture with a Degradable Coating

The coated suture of Example 32 is reattached to the overhead stirrer and is rotated at 30 rpm. A 10% 20:80 MePEG(750)—PLA block copolymer solution (acetone) is sprayed onto the rotating coated suture using a TLC spray device. After the spraying process, the suture is allowed to air dry for 30 minutes while still rotating. The spray coating process can be repeated until the desired thickness or uniformity of coating is obtained. The suture is then removed from the rod and is further dried under vacuum for 24 h.

### Example 30

### Coating of a Suture with a Powdered Silk/Cyclosporine A/Polyester Coating

A monofilament polydioxanone suture (size 3-0), either self-retaining or not self-retaining, is attached to a stainless steel rod (with the aid of a bulldog clip), which is attached to a Fisher overhead stirrer that is orientated vertically. The stirrer is set to rotate at 30 rpm. A low molecular weight glycolide/ε-caprolactone copolymer (10:90 w/w) in methylene chloride that contains the powdered silk and Cyclosporine A is sprayed onto the rotating suture using a TLC spray device. The concentration of the powdered silk in the polyurethane solution is altered from 0.1% to 50% (w/w relative to the polymer) and the concentration of the Cyclosporine A is altered from 0.1% to 10% (w/w relative to the polymer). After the spraying process, the suture is allowed to air dry for 30 minutes while still rotating. The suture is then removed from the rod and is further dried under vacuum for 24 h.

### Example 31

### Coating of a Suture with Cyclosporine A

A 5% low molecular weight glycolide/ε-caprolactone copolymer (10:90 w/w) in methylene chloride containing from 0.1% to 10% cyclosporine A is prepared. A piece PROLENE suture (Johnson & Johnson Corporation, New Brunswick, N.J.) is immersed in and then withdrawn from the coating solution. The coated sample is air-dried in the fume-hood. Samples of different coating thicknesses are prepared by repeating the dip-coating process. The coated sample is then dried under vacuum for 24 hours.

### Example 32

### Collagen Synthesis Assay

An in vitro assay is described for determining whether a substance promotes deposition of extracellular matrix (ECM). Normal human dermal fibroblasts are trypsinized, then re-plated in medium containing ascorbic acid-2-phosphate at 150,000 cells per well in a 12-well plate. The cells are cultured at 37° C. and 5% CO₂ for 2-3 weeks with media changes every three days so that they formed a 3-D matrix of cells and collagen. After 14-21 days of culture, the medium is replaced with serum free medium and the cells allowed to rest for 24 hours.

Drug is diluted in DMSO at 10⁻²M, and then diluted 10 fold to give a range of stock concentrations from 10⁻²M to 10⁻⁸M. Drug is then diluted 1000 times in fresh serum free medium and added to the wells in a total volume of 3 ml per well. The plate(s) are then incubated for 72 hrs at 37° C. After 72 hrs the media is removed from the wells and put into microcentrifuge tubes and frozen at −20° C. until assayed.

The amount of collagen synthesized is measured using a Procollagen Type 1 C-Peptide (PIP) EIA kit (Takara Bio Inc., Shiga, Japan), where the amount of collagen produced is stoichiometrically represented by the amount of pro-peptide cleaved from the collagen when it is secreted. Anti-PIP monoclonal antibodies are immobilized on an ELISA plate, the samples added, then a second PIP monoclonal antibody conjugated to horseradish peroxidase is added to the wells and incubated. Following incubation, the wells are washed, a substrate solution is added, and the absorbance measured in a plate reader at 450 nm and compared to a standard curve of PIP (ng/ml).

### Example 33

### In Vivo Evaluation of Perivascular PU Films Coated with Degummed or Virgin Silk Strands

Wistar rats weighing 300 g to 400 g were anesthetized with halothane. The skin over the neck region is shaved and the skin was sterilized. A vertical incision was made over the trachea and the left carotid artery is exposed. A polyurethane film covered with degummed silk strands, virgin silk strands, or a control uncoated PU film was wrapped around a distal segment of the common carotid artery. The wound was closed and the animal was allowed to recover.

After 28 days, the rats were sacrificed with carbon dioxide and then pressure-perfused at 100 mmHg with 10% buffered formaldehyde. Both carotid arteries were harvested and processed for histology. Serial cross-sections were cut every 2 mm in the treated left carotid artery and at corresponding levels in the untreated right carotid artery. Sections were stained with H&E and Movat's stains to evaluate tissue growth around the carotid artery. Thickness of perivascular granulation tissue was quantified by computer-assisted morphometric analysis.

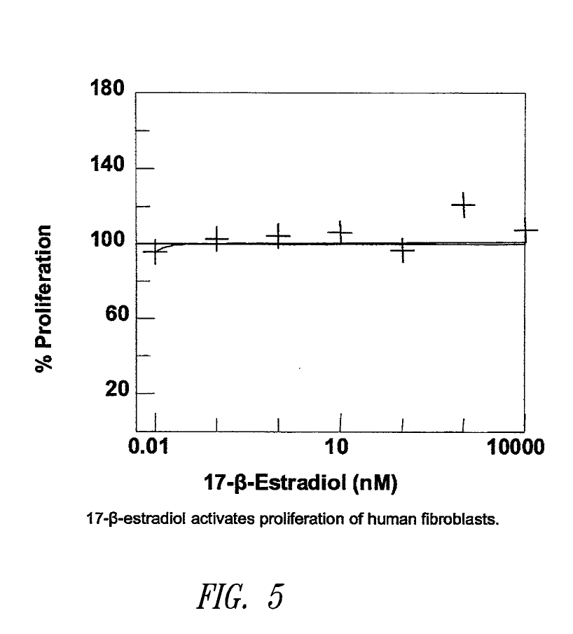

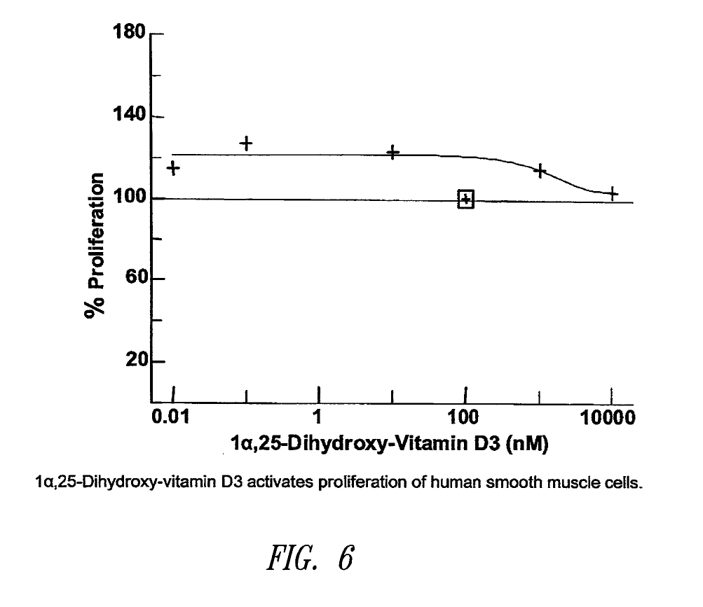

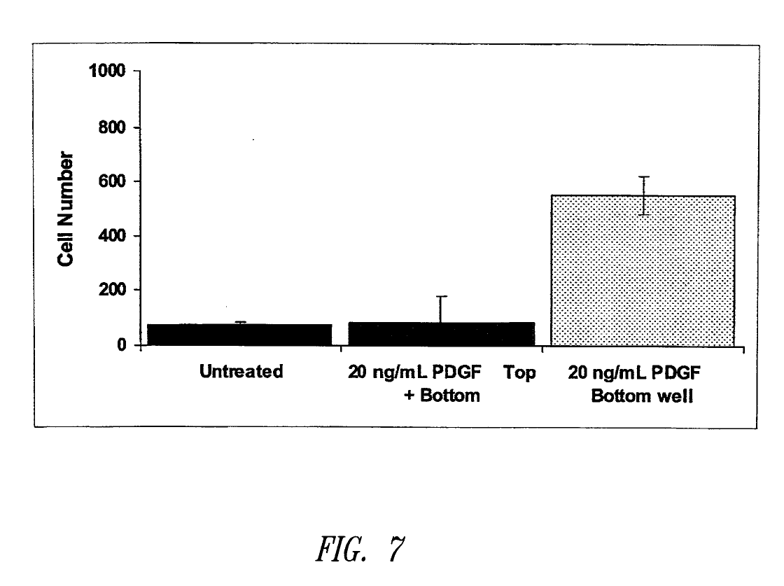

Both types of silk markedly increased granulation tissue growth around the blood vessel to the same extent. The silk strands in both groups had broken down into small particles (approximately 30 um in diameter) scattered around the blood vessel and were surrounded by giant cells, macrophages, proteoglycan matrix, and blood vessels. These features are typical of a foreign body response. The area covered by the foreign body response was more variable in the virgin silk group than in the degummed silk group. See FIGS. 5, 6, and 7.

### Example 34

### MIC (Minimum Inhibitory Concentration) Determination by Microtitre Broth Dilution Method

### A. MIC Assay of Various Gram Negative and Positive Bacteria

MIC assays were conducted essentially as described by Amsterdam, D. 1996, “Susceptibility testing of antimicrobials in liquid media,” p. 52-111. In Loman, V., ed. Antibiotics in Laboratory Medicine, 4th ed. Williams and Wilkins, Baltimore, Md. Briefly, a variety of compounds were tested for antibacterial activity against isolates of P. aeruginosa, K. pneumoniae , E. coli, S. epidermidus, and S. aureus in the MIC (minimum inhibitory concentration) assay under aerobic conditions using 96 well polystyrene microtitre plates (Falcon 1177), and Mueller Hinton broth at 37° C. incubated for 24 h. (MHB was used for most testing except C721 (S. pyogenes), which used Todd Hewitt broth, and Haemophilus influenzae, which used Haemophilus test medium (HTM)). Tests were conducted in triplicate. The results are provided below in Table 1.

TABLE 1
MINIMUM INHIBITORY CONCENTRATIONS OF THERAPEUTIC AGENTS
AGAINST VARIOUS GRAM NEGATIVE AND POSITIVE BACTERIA

| | Bacterial Strain | | | | | |
|---|---|---|---|---|---|---|
| | P. aeruginosa | K. pneumoniae | E. Coli | S. aureus | | |
| | PAE/K799 | ATCC13883 | UB1005 | ATCC25923 | S. epidermidis | S. pyogenes |
| | H187 | C238 | C498 | C622 | C621 | C721 |
| | wt | wt | wt | wt | wt | wt |
| Drug | Gram − | Gram − | Gram − | Gram + | Gram + | Gram + |
| doxorubicin | 10⁻⁵ | 10⁻⁶ | 10⁻⁴ | 10⁻⁵ | 10⁻⁶ | 10⁻⁷ |
| mitoxantrone | 10⁻⁵ | 10⁻⁶ | 10⁻⁵ | 10⁻⁵ | 10⁻⁵ | 10⁻⁶ |
| 5-fluorouracil | 10⁻⁵ | 10⁻⁶ | 10⁻⁶ | 10⁻⁷ | 10⁻⁷ | 10⁻⁴ |
| methotrexate | N | 10⁻⁶ | N | 10⁻⁵ | N | 10⁻⁶ |
| etoposide | N | 10⁻⁵ | N | 10⁻⁵ | 10⁻⁶ | 10⁻⁵ |
| camptothecin | N | N | N | N | 10⁻⁴ | N |
| hydroxyurea | 10⁻⁴ | N | N | N | N | 10⁻⁴ |
| cisplatin | 10⁻⁴ | N | N | N | N | N |
| tubercidin | N | N | N | N | N | N |
| 2-mercaptopurine | N | N | N | N | N | N |
| 6-mercaptopurine | N | N | N | N | N | N |
| Cytarabine | N | N | N | N | N | N |

MICs are presented in molar concentrations.
Wt = wild type
N = No activity

### B. MIC of Antibiotic-Resistant Bacteria

Various concentrations of the following compounds, mitoxantrone, cisplatin, tubercidin, methotrexate, 5-fluorouracil, etoposide, 2-mercaptopurine, doxorubicin, 6-mercaptopurine, camptothecin, hydroxyurea and cytarabine were tested for antibacterial activity against clinical isolates of a methicillin resistant S. aureus and a vancomycin-resistant pediocoocus clinical isolate in an MIC assay as described above. Compounds which showed inhibition of growth (MIC value of <1.0×10⁻³) of these resistant bacterial strains included mitoxantrone (both strains); methotrexate (vancomycin resistant pediococcus); 5-fluorouracil (both strains); etoposide (both strains); and 2-mercaptopurine (vancomycin resistant pediococcus).

Additional agents that may be tested in this assay include but are not limited to fourth generation penicillins such as mezlocillin and piperacillin (the ureidopenicillins), and carbenicillin and ticarcillin (carboxypenicillins), and analogues and derivatives thereof; first generation cephalosporins such as cephazolin sodium, cephalexin (Keflex), cefazolin (Ancef), cephapirin (Cefadyl) and cephalothin (Keflin) and analogues and derivatives thereof; Ticarcillin, second generation cephalosporins such as cefuroxime (Zinocef), cefotetan (Cefotan), and cefoxitin (Mefoxin) and analogues and derivatives thereof; third generation cephalosporin such as Naxcel (Ceftiofur Sodium), Cefdinir (Omnicef), cefoperazone (Cefobid), ceftazidime (Fortaz), ceftriaxone (Rocephin), cefotaxime (Claforan) and analogues and derivatives thereof; fourth generation cephalosporins such as cefepime (Maxipime) and analogues and derivatives thereof; monobactams such as aztreonam and analogues and derivatives thereof; carbapenems such as imipenem, ertapenem and meropenem and analogues and derivatives thereof; inhibitors of protein synthesis such as aminoglycosides including streptomycin, gentamicin, tobramycin, and amikacin and analogues and derivatives thereof; inhibitors of protein synthesis such as the MSL group including macrolides (Erythromycin), long acting macrolides (Azithromycin) and lincosamides (Clindamycin) and streptogramins (Syneroid), clarithromycin, kanamycin Sulfate, and analogues and derivatives thereof; inhibitors of DNA synthesis such as the quinolones including ciprofloxacin, ofloxacin, gatifloxacin, moxifloxacin, levofloxacin, trovafloxacin and analogues and derivatives thereof; inhibitors of DNA synthesis such as metronidazole and analogues and derivatives thereof; inhibitors of folate metabolism including sulfonamides and trimethoprim and analogues and derivatives thereof. Additional agents include but are not limited to cefixime, spectinomycin, tetracycline, nitrofurantoin, doxycycline, polymyxin B, neomycin sulfate, and analogues and derivatives thereof.

### Example 35

### Screening Assay for Assessing the Effect of Cyclosporine a on Cell Proliferation

An in vitro assay is described below for determining whether a substance stimulates cell (e.g., fibroblast) proliferation (see, e.g., In Vitro Toxicol. (1990) 3:219; Biotech. Histochem. (1993) 68: 29; Anal. Biochem. (1993) 213:426). Primary human smooth muscle cells were cultured according to standard cell culture methods. When the cells were at 70-90% confluency, the cells were trypsinized and replated at 600 cells/well in media in 96-well plates and allowed to attach overnight. Cyclosporine A was prepared in DMSO at a concentration of 10⁻² M and then diluted in 10-fold dilutions to give a range of stock concentrations (10⁻⁸ M to 10⁻² M). Each drug dilution was diluted 1/1000 in media and added to cells to give a total volume of 200 μL/well. Each drug concentration was tested in triplicate wells. Plates containing smooth muscle cells and cyclosporine A were incubated at 37° C. for 72 hours.

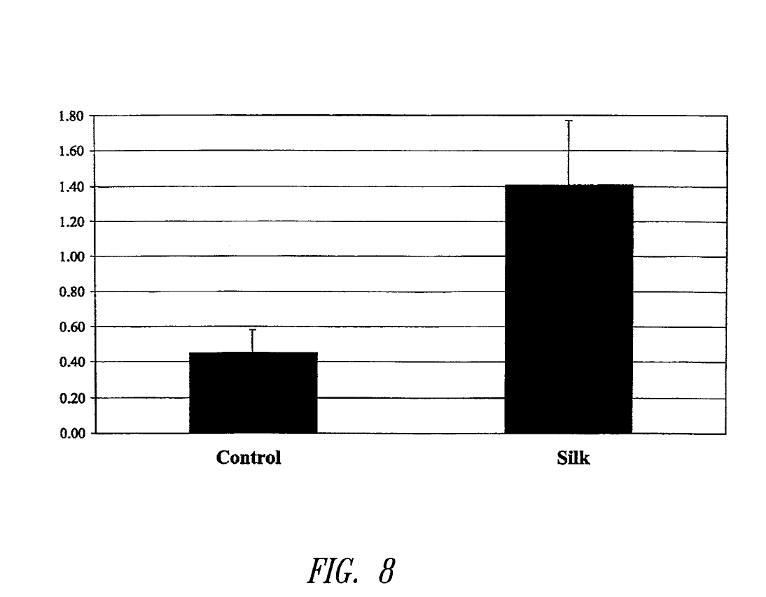

To terminate the assay, the media was removed by gentle aspiration. A 1/400 dilution of CYQUANT 400×GR dye indicator (Molecular Probes, Eugene, Oreg.) was added to 1× cell lysis buffer, and 200 μl of the mixture was added to the wells of the plate. Plates were incubated at room temperature, protected from light for 3-5 minutes. Fluorescence was read in a fluorescence microplate reader at ˜480 nm excitation wavelength and ˜520 nm emission maxima. Activation of proliferation was determined by taking the average of triplicate wells and comparing average relative fluorescence units to the DMSO control. Data from a representative assay are shown in FIG. 8.

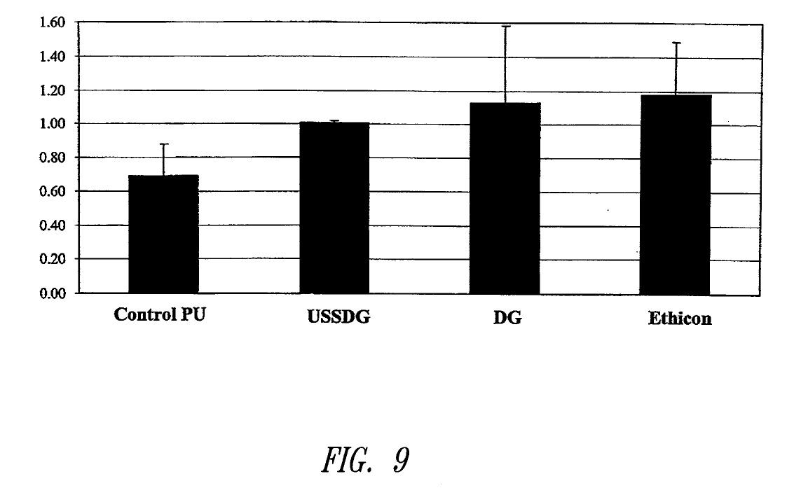

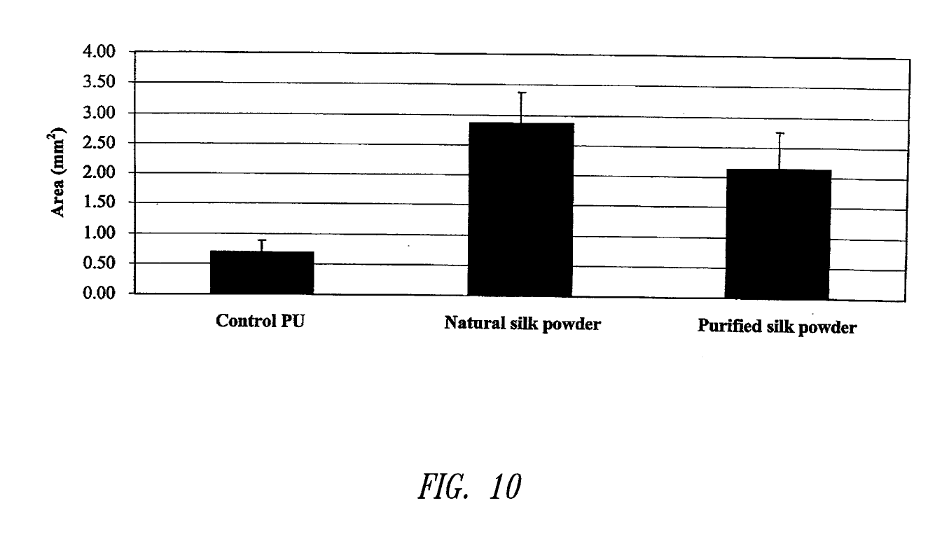

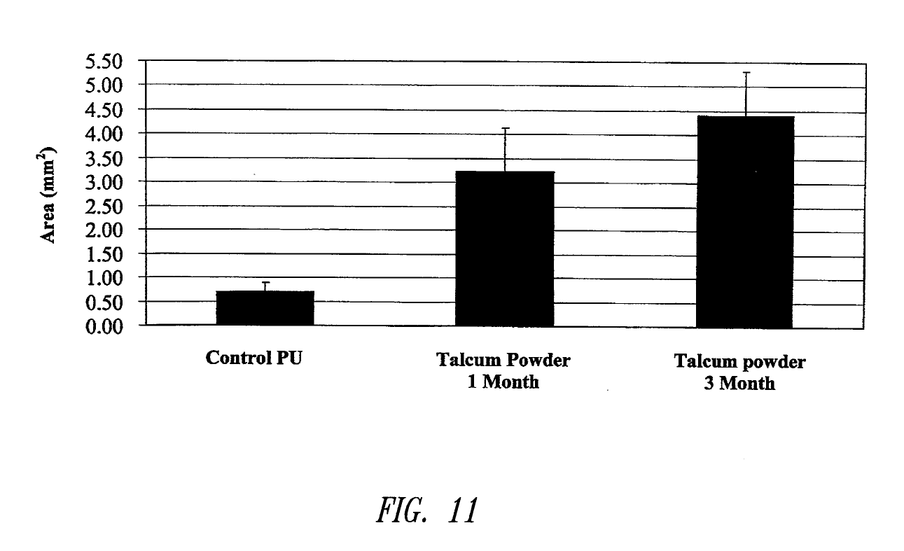

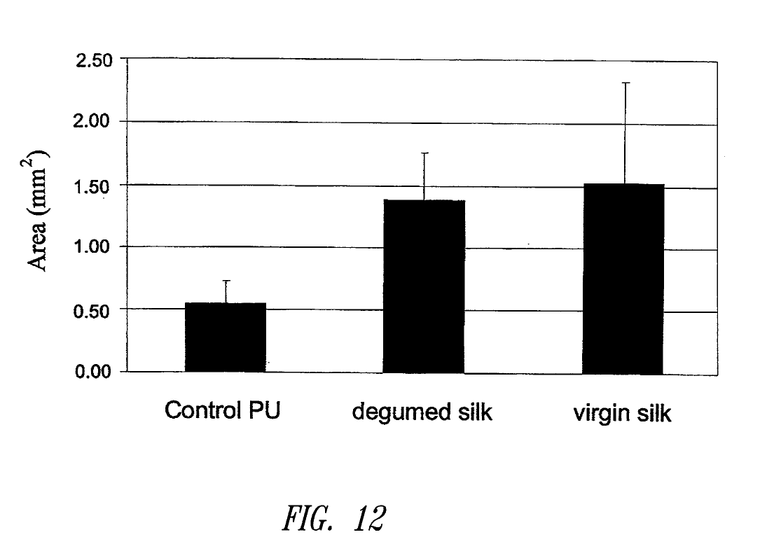

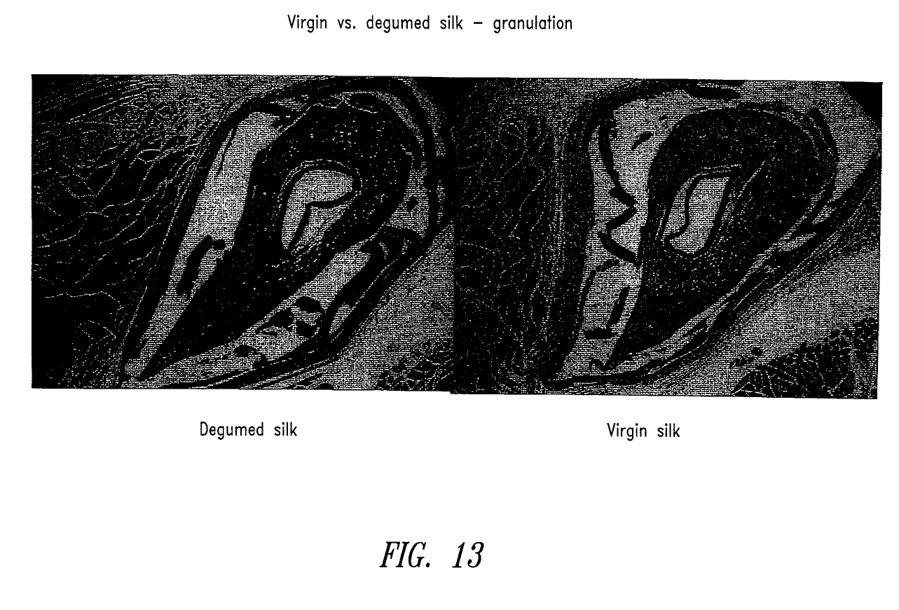

The assay was repeated for the following proliferative therapeutic agents using smooth muscle cells or human fibroblasts; data are presented in the figures as noted: dexamethasone (FIG. 9), all-trans retinoic acid (FIG. 10), isotretinoin (FIG. 11), 17-β-estradiol (FIG. 12), and 1α,25-dihydroxy-vitamin D₃ (FIG. 13).

### Example 36

### Screening Assay for Assessing the Effect of PDGF on Smooth Muscle Cell Migration

An in vitro assay was described for determining whether a substance stimulates cell (e.g., fibroblast) migration. Primary human smooth muscle cells were starved of serum in smooth muscle cell basal media containing insulin and human basic fibroblast growth factor (bFGF) for 16 hours prior to the assay. For the migration assay, cells were trypsinized to remove cells from flasks, washed with migration media, and diluted to a concentration of 2-2.5×10⁵ cells/ml in migration media. Migration media consisted of phenol red free Dulbecco's Modified Eagle Medium (DMEM) containing 0.35% human serum albumin. A 100 μl volume of smooth muscle cells (approximately 20,000-25,000 cells) was added to the top of a Boyden chamber assembly (Chemicon QCM CHEMOTAXIS 96-well migration plate). To the bottom wells, the chemotactic agent, recombinant human platelet derived growth factor (rhPDGF-BB), was added at a concentration of 10 ng/ml in a total volume of 150 μL. Paclitaxel was prepared in DMSO at a concentration of 10⁻²M and serially diluted 10-fold to give a range of stock concentrations (10⁻⁸ M to 10⁻² M). Paclitaxel was added to cells by directly adding paclitaxel DMSO stock solutions, prepared earlier, at a 1/1000 dilution, to the cells in the top chamber. Plates were incubated for 4 hours to allow cell migration.

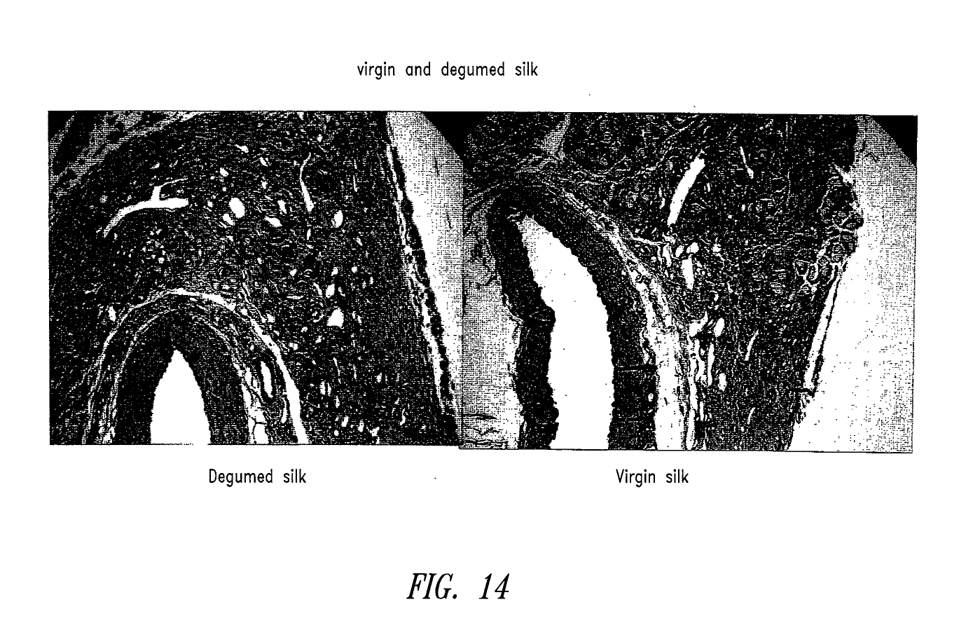

At the end of the 4 hour period, cells in the top chamber were discarded, and the smooth muscle cells attached to the underside of the filter were detached for 30 minutes at 37° C. in Cell Detachment Solution (Chemicon). Dislodged cells were lysed in lysis buffer containing the DNA binding CYQUANT GR dye and incubated at room temperature for 15 minutes. Fluorescence was read in a fluorescence microplate reader at ˜480 nm excitation wavelength and ˜520 nm emission maxima. Relative fluorescence units from triplicate wells were averaged after subtracting background fluorescence (control chamber without chemoattractant), and average number of cells migrating was obtained from a standard curve of smooth muscle cells serially diluted from 25,000 cells/well down to 98 cells/well. Inhibitory concentration of 50% (IC₅₀) was determined by comparing the average number of cells migrating in the presence of paclitaxel to the positive control (smooth muscle cell chemotaxis in response to rhPDGF-BB). The results of a representative assay were shown in FIG. 14. (See also references: Biotechniques 29:81 (2000); J. Immunol. Methods 254:85 (2001)).

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A suture comprising at least one well containing a fibrosing agent or a composition comprising a fibrosing agent.

2. The suture of claim 1 wherein the suture is a self-retaining suture.

3-6. (canceled)

7. The suture of claim 1 wherein the fibrosing agent is a tissue irritant.

8. The suture of claim 7 wherein the tissue irritant is talcum powder, metallic beryllium and oxides thereof, copper, silk, fibroin, sericin, wool, silica, crystalline silicates, talc, quartz dust, inflammatory microcrystals, or ethanol.

9. The suture of claim 8 wherein the tissue irritant is silk.

10. (canceled)

11. (canceled)

12. The suture of claim 9 wherein the silk is spider silk, silkworm silk, raw silk, degummed silk, milled silk, sprayed silk, powdered silk, silk protein, modified silk, hydrolyzed silk, silk amino acids, or cocodiammonium hydroxypropyl silk amino acid.

13. (canceled)

14. The suture of claim 1 wherein the fibrosing agent is a component of extracellular matrix, an inflammatory cytokine, a growth hormone, a bone morphogenic protein, an adhesive, bleomycin or an analog or derivative thereof, polylysine, poly(ethylene-co-vinylacetate), chitosan, N-carboxybutylchitosan, a peptide or protein having an RGD sequence, or a polymer of vinyl chloride.

15-22. (canceled)

23. The suture of claim 1 wherein the fibrosing agent or composition comprising a fibrosing agent is in the form of microparticles, nanoparticles, microspheres, microcapsules, pastes, gels, sprays, or liquids.

24. The suture of claim 1, further comprising a second pharmaceutically active agent.

25-29. (canceled)

30. The suture of claim 1, the at least one well further comprising a polymer.

31-41. (canceled)

42. The suture of claim 1 wherein the fibrosing agent is released from the suture in concentrations effective to induce a fibrotic response in tissue proximate to the suture after deployment of the suture.

43-48. (canceled)

49. The suture of claim 1 wherein the fibrosing agent is not released from the suture into tissue proximate to the suture after deployment of the suture.

50-60. (canceled)

61. A method for making the suture of claim 1, comprising: combining a suture with a fibrosing agent, wherein the fibrosing agent induces or stimulates a fibrotic response between the suture and tissue or between portions or layers of tissue of a patient in which the suture is implanted.

62-65. (canceled)

66. A method for repositioning tissue, comprising: implanting a suture of claim 1 into a tissue in need of repositioning and moving the suture such that the tissue into which the suture is implanted is repositioned, wherein the fibrosing agent induces or stimulates a fibrotic response between the suture and the tissue in contact with the suture and/or between portions or layers of tissue into which the suture is implanted.

67-72. (canceled)

73. A method for joining or connecting portions or layers of tissue in a patient in need thereof, comprising: joining or connecting surfaces or edges of the portions or layers of tissue with a suture of claim 1, wherein the fibrosing agent induces or stimulates a fibrotic response between the suture and the tissue in contact with the suture and/or between the portions or layers of tissue.

74. The method of claim 73 wherein the surfaces or edges of portions or layers of tissue are surfaces or edges of a wound.

75-92. (canceled)

93. A method for stabilizing an internal structure within a body, comprising:
placing a suture of claim 1 into a laparoscopic insertion device;
inserting the insertion device through an abdominal wall;
further inserting the insertion device into the internal structure in need of stabilization; and
withdrawing the insertion device;
wherein an end portion of the suture remains attached to the internal structure in need of stabilization and another end portion of the suture remains attached to the abdominal wall, thus stabilizing the internal structure.

94-103. (canceled)

104. A method for attaching or securing a foreign element or device to a body tissue, comprising attaching the foreign element or device to the tissue using a suture of claim 1.

105-122. (canceled)

123. A method for fastening an endoluminal organ or a portion thereof, comprising: tying the endoluminal organ or portion thereof with a suture of claim 1.

124. (canceled)
